SEQUENCE LISTING (165 sequences)

SEQ ID NO: 1 - protein, 120 aa, synthetic construct
QVTLKESGGGLVRTGGSLRLSCAASGSISIFSMYVVAWYRQAPGKQREWVAFIQRGGTANYADSAKGRFTISRDNAKNTVYLQMASLKPDDTAVYYCNYINSDNEIGGPWGQGTQVTVKP

SEQ ID NO: 2 - protein, 12 aa, synthetic construct
GSISIFSMYVVA

SEQ ID NO: 3 - protein, 9 aa, synthetic construct
FIQRGGTAN

SEQ ID NO: 4 - protein, 10 aa, synthetic construct
INSDNEIGGP

SEQ ID NO: 5 - protein, 120 aa, synthetic construct
EVQLLESGGGEVQPGGSLRLSCAASGSISIFSMYVVAWYRQAPGKEREWVAFIQRGGTANYAESAKGRFTISRDNAKNTVYLQMSSLRAEDTAVYYCNYINTDNEIGGPWGQGTLVTVKP

SEQ ID NO: 6 - protein, 12 aa, synthetic construct
GSISIFSMYVVA

SEQ ID NO: 7 - protein, 9 aa, synthetic construct
FIQRGGTAN

SEQ ID NO: 8 - protein, 10 aa, synthetic construct
INTDNEIGGP

SEQ ID NO: 9 - protein, 246 aa, synthetic construct
EVQLLESGGGEVQPGGSLRLSCAASGSISIFSMYVVAWYRQAPGKEREWVAFIQRGGTANYAESAKGRFTISRDNAKNTVYLQMSSLRAEDTAVYYCNYINTDNEIGGPWGQGTLVTVKPGGSGGSEVQLLESGGGEVQPGGSLRLSCAASGSISIFSMYVVAWYRQAPGKEREWVAFIQRGGTANYAESAKGRFTISRDNAKNTVYLQMSSLRAEDTAVYYCNYINTDNEIGGPWGQGTLVTVKP

SEQ ID NO: 10 - protein, 372 aa, synthetic construct
EVQLLESGGGEVQPGGSLRLSCAASGSISIFSMYVVAWYRQAPGKEREWVAFIQRGGTANYAESAKGRFTISRDNAKNTVYLQMSSLRAEDTAVYYCNYINTDNEIGGPWGQGTLVTVKPGGSGGSEVQLLESGGGEVQPGGSLRLSCAASGSISIFSMYVVAWYRQAPGKEREWVAFIQRGGTANYAESAKGRFTISRDNAKNTVYLQMSSLRAEDTAVYYCNYINTDNEIGGPWGQGTLVTVKPGGSGGSEVQLLESGGGEVQPGGSLRLSCAASGSISIFSMYVVAWYRQAPGKEREWVAFIQRGGTANYAESAKGRFTISRDNAKNTVYLQMSSLRAEDTAVYYCNYINTDNEIGGPWGQGTLVTVKP

SEQ ID NO: 11 - protein, 120 aa, synthetic construct
EVQLLESGGGEVQPGGSLRLSCAASGSISIFSDYVVAWYRQAPGKEREWVAFIQRGGTANYAESAKGRFTISRDNAKNTVYLQMSSLRAEDTAVYYCNYINTDNEIGGPWGQGTLVTVRP

SEQ ID NO: 12 - protein, 246 aa, synthetic construct
EVQLLESGGGEVQPGGSLRLSCAASGSISIFSDYVVAWYRQAPGKEREWVAFIQRGGTANYAESAKGRFTISRDNAKNTVYLQMSSLRAEDTAVYYCNYINTDNEIGGPWGQGTLVTVRPGGSGGSEVQLLESGGGEVQPGGSLRLSCAASGSISIFSDYVVAWYRQAPGKEREWVAFIQRGGTANYAESAKGRFTISRDNAKNTVYLQMSSLRAEDTAVYYCNYINTDNEIGGPWGQGTLVTVRP

SEQ ID NO: 13 - protein, 12 aa, synthetic construct
GSISIFSDYVVA

SEQ ID NO: 14 - protein, 9 aa, synthetic construct
FIQRGGTAN

SEQ ID NO: 15 - protein, 10 aa, synthetic construct
INTDNEIGGP

SEQ ID NO: 16 - protein, 120 aa, synthetic construct
EVQLLESGGGEVQPGGSLRLSCAASGSISIFSKYVVAWYRQAPGKEREWVAFIQRGGTANYAESAKDRFTISRDNAKNTVYLQMSSLRAEDTAVYYCNYINTDNEIGGPWGQGTLVTVRP

SEQ ID NO: 17 - protein, 246 aa, synthetic construct
EVQLLESGGGEVQPGGSLRLSCAASGSISIFSKYVVAWYRQAPGKEREWVAFIQRGGTANYAESAKDRFTISRDNAKNTVYLQMSSLRAEDTAVYYCNYINTDNEIGGPWGQGTLVTVRPGGSGGSEVQLLESGGGEVQPGGSLRLSCAASGSISIFSKYVVAWYRQAPGKEREWVAFIQRGGTANYAESAKDRFTISRDNAKNTVYLQMSSLRAEDTAVYYCNYINTDNEIGGPWGQGTLVTVRP

SEQ ID NO: 18 - protein, 12 aa, synthetic construct
GSISIFSKYVVA

SEQ ID NO: 19 - protein, 9 aa, synthetic construct
FIQRGGTAN

SEQ ID NO: 20 - protein, 10 aa, synthetic construct
INTDNEIGGP

SEQ ID NO: 21 - protein, 120 aa, synthetic construct
EVQLLESGGGEVQPGGSLRLSCAASGSISIFSKYVVAWYRQAPGKEREWVAFIQRGGTANYAESAKGRFTISRDNAKNTVYLQMSSLRAEDTAVYYCNYINTDNEIGGPWGQGTLVTVEP

SEQ ID NO: 22 - protein, 246 aa, synthetic construct
EVQLLESGGGEVQPGGSLRLSCAASGSISIFSKYVVAWYRQAPGKEREWVAFIQRGGTANYAESAKGRFTISRDNAKNTVYLQMSSLRAEDTAVYYCNYINTDNEIGGPWGQGTLVTVEPGGSGGSEVQLLESGGGEVQPGGSLRLSCAASGSISIFSKYVVAWYRQAPGKEREWVAFIQRGGTANYAESAKGRFTISRDNAKNTVYLQMSSLRAEDTAVYYCNYINTDNEIGGPWGQGTLVTVEP

SEQ ID NO: 23 - protein, 12 aa, synthetic construct
GSISIFSKYVVA

SEQ ID NO: 24 - protein, 9 aa, synthetic construct
FIQRGGTAN

SEQ ID NO: 25 - protein, 10 aa, synthetic construct
INTDNEIGGP

SEQ ID NO: 26 - protein, 120 aa, synthetic construct
EVQLLESGGGEVQPGGSLRLSCAASGSISIFSKYVVAWYRQAPGKEREWVAFIQSGGTANYAESAKGRFTISRDNAKNTVYLQMSSLRAEDTAVYYCNYINTDNEIGGPWGQGTLVTVRP

SEQ ID NO: 27 - protein, 246 aa, synthetic construct
EVQLLESGGGEVQPGGSLRLSCAASGSISIFSKYVVAWYRQAPGKEREWVAFIQSGGTANYAESAKGRFTISRDNAKNTVYLQMSSLRAEDTAVYYCNYINTDNEIGGPWGQGTLVTVRPGGSGGSEVQLLESGGGEVQPGGSLRLSCAASGSISIFSKYVVAWYRQAPGKEREWVAFIQSGGTANYAESAKGRFTISRDNAKNTVYLQMSSLRAEDTAVYYCNYINTDNEIGGPWGQGTLVTVRP

SEQ ID NO: 28 - protein, 12 aa, synthetic construct
GSISIFSKYVVA

SEQ ID NO: 29 - protein, 9 aa, synthetic construct
FIQSGGTAN

SEQ ID NO: 30 - protein, 10 aa, synthetic construct
INTDNEIGGP

SEQ ID NO: 31 - protein, 120 aa, synthetic construct
QVQLLESGGGEVQPGGSLRLSCAASGSISIFSKYVVAWYRQAPGKEREWVAFIQRGGTANYAESAKGRFTISRDNAKNTVYLQMSSLRAEDTAVYYCNYINTDNEIGGPWGQGTLVTVEP

SEQ ID NO: 32 - protein, 246 aa, synthetic construct
QVQLLESGGGEVQPGGSLRLSCAASGSISIFSKYVVAWYRQAPGKEREWVAFIQRGGTANYAESAKGRFTISRDNAKNTVYLQMSSLRAEDTAVYYCNYINTDNEIGGPWGQGTLVTVEPGGSGGSQVQLLESGGGEVQPGGSLRLSCAASGSISIFSKYVVAWYRQAPGKEREWVAFIQRGGTANYAESAKGRFTISRDNAKNTVYLQMSSLRAEDTAVYYCNYINTDNEIGGPWGQGTLVTVEP

SEQ ID NO: 33 - protein, 12 aa, synthetic construct
GSISIFSKYVVA

SEQ ID NO: 34 - protein, 9 aa, synthetic construct
FIQRGGTAN

SEQ ID NO: 35 - protein, 10 aa, synthetic construct
INTDNEIGGP

SEQ ID NO: 36 - protein, 120 aa, synthetic construct
QVQLLESGGGEVQPGGSLRLSCAASGSISIFSDYVVAWYRQAPGKEREWVAFIQRGGTANYAESAKGRFTISRDNAKNTVYLQMSSLRAEDTAVYYCNYINTDNEIGGPWGQGTLVTVRP

SEQ ID NO: 37 - protein, 246 aa, synthetic construct
QVQLLESGGGEVQPGGSLRLSCAASGSISIFSDYVVAWYRQAPGKEREWVAFIQRGGTANYAESAKGRFTISRDNAKNTVYLQMSSLRAEDTAVYYCNYINTDNEIGGPWGQGTLVTVRPGGSGGSQVQLLESGGGEVQPGGSLRLSCAASGSISIFSDYVVAWYRQAPGKEREWVAFIQRGGTANYAESAKGRFTISRDNAKNTVYLQMSSLRAEDTAVYYCNYINTDNEIGGPWGQGTLVTVRP

SEQ ID NO: 38 - protein, 12 aa, synthetic construct
GSISIFSDYVVA

SEQ ID NO: 39 - protein, 9 aa, synthetic construct
FIQRGGTAN

SEQ ID NO: 40 - protein, 10 aa, synthetic construct
INTDNEIGGP

SEQ ID NO: 41 - protein, 580 aa, synthetic construct
MEGDRQYGDGYLLQVQELVTVQEGLCVHVPCSFSYPQDGWTDSDPVHGYWFRAGDRPYQDAPVATNNPDREVQAETQGRFQLLGDIWSNDCSLSIRDARKRDKGSYFFRLERGSMKWSYKSQLNYKTKQLSVFVTALTHRPDILILGTLESGHSRNLTCSVPWACKQGTPPMISWIGASVSSPGPTTARSSVLTLTPKPQDHGTSLTCQVTLPGTGVTTTSTVRLDVSYPPWNLTMTVFQGDATASTALGNGSSLSVLEGQSLRLVCAVNSNPPARLSWTRGSLTLCPSRSSNPGLLELPRVHVRDEGEFTCRAQNAQGSQHISLSLSLQNEGTGTSRPVSQVTLAAGTGGSGGGGCPPCPAPELPGGPSVFVFPPKPKDVLSISGRPEVTCVVVDVGKEDPEVNFNWYIDGVEVRTANTKPKEEQFNSTYRVVSVLPIQHQDWLTGKEFKCKVNNKALPAPIERTISKAKGQTREPQVYTLAPHREELAKDTVSVTCLVKGFYPADINVEWQRNGQPESEGTYANTPPQLDNDGTYFLYSKLSVGKNTWQRGETLTCVVMHEALHNHYTQKSISQSLGK

SEQ ID NO: 42 - protein, 499 aa, Homo sapiens
MLLLLLLLPLLWGTKGMEGDRQYGDGYLLQVQELVTVQEGLCVHVPCSFSYPQDGWTDSDPVHGYWFRAGDRPYQDAPVATNNPDREVQAETQGRFQLLGDIWSNDCSLSIRDARKRDKGSYFFRLERGSMKWSYKSQLNYKTKQLSVFVTALTHRPDILILGTLESGHSRNLTCSVPWACKQGTPPMISWIGASVSSPGPTTARSSVLTLTPKPQDHGTSLTCQVTLPGTGVTTTSTVRLDVSYPPWNLTMTVFQGDATASTALGNGSSLSVLEGQSLRLVCAVNSNPPARLSWTRGSLTLCPSRSSNPGLLELPRVHVRDEGEFTCRAQNAQGSQHISLSLSLQNEGTGTSRPVSQVTLAAVGGAGATALAFLSFCIIFIIVRSCRKKSARPAAGVGDTGMEDAKAIRGSASQGPLTESWKDGNPLKKPPPAVAPSSGEEGELHYATLSFHKVKPQDPQGQEATDSEYSEIKIHKRETAETQACLRNHNPSSKEVRG

SEQ ID NO: 43 - protein, 483 aa, Homo sapiens
MEGDRQYGDGYLLQVQELVTVQEGLCVHVPCSFSYPQDGWTDSDPVHGYWFRAGDRPYQDAPVATNNPDREVQAETQGRFQLLGDIWSNDCSLSIRDARKRDKGSYFFRLERGSMKWSYKSQLNYKTKQLSVFVTALTHRPDILILGTLESGHSRNLTCSVPWACKQGTPPMISWIGASVSSPGPTTARSSVLTLTPKPQDHGTSLTCQVTLPGTGVTTTSTVRLDVSYPPWNLTMTVFQGDATASTALGNGSSLSVLEGQSLRLVCAVNSNPPARLSWTRGSLTLCPSRSSNPGLLELPRVHVRDEGEFTCRAQNAQGSQHISLSLSLQNEGTGTSRPVSQVTLAAVGGAGATALAFLSFCIIFIIVRSCRKKSARPAAGVGDTGMEDAKAIRGSASQGPLTESWKDGNPLKKPPPAVAPSSGEEGELHYATLSFHKVKPQDPQGQEATDSEYSEIKIHKRETAETQACLRNHNPSSKEVRG

SEQ ID NO: 44 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - protein, 224 aa, Homo sapiens
DKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 12 aa, synthetic construct
  VARIANT 8: note = M, K, D, Y, L, or I
GSISIFSXYVVA

SEQ ID NO: 47 - protein, 9 aa, synthetic construct
  VARIANT 4: note = R or S
FIQXGGTAN

SEQ ID NO: 48 - protein, 10 aa, synthetic construct
  VARIANT 2: note = N, Q, S, or T
  VARIANT 3: note = S or T
IXXDNEIGGP

SEQ ID NO: 49 - protein, 119 aa, synthetic construct
QVQVVESGGGLVQPGGSLRLSCAASGSTSLLNAMAWYRQAPGKERELVAAISSGGGTYYADSVKGRFTISTNNAKNTLYLQMNSLKPEDTAMYYCAPGPNYSDYEEINWGQGTQVTVKP

SEQ ID NO: 50 - protein, 244 aa, synthetic construct
QVQVVESGGGLVQPGGSLRLSCAASGSTSLLNAMAWYRQAPGKERELVAAISSGGGTYYADSVKGRFTISTNNAKNTLYLQMNSLKPEDTAMYYCAPGPNYSDYEEINWGQGTQVTVKPGGSGGSQVQVVESGGGLVQPGGSLRLSCAASGSTSLLNAMAWYRQAPGKERELVAAISSGGGTYYADSVKGRFTISTNNAKNTLYLQMNSLKPEDTAMYYCAPGPNYSDYEEINWGQGTQVTVKP

SEQ ID NO: 51 - protein, 369 aa, synthetic construct
QVQVVESGGGLVQPGGSLRLSCAASGSTSLLNAMAWYRQAPGKERELVAAISSGGGTYYADSVKGRFTISTNNAKNTLYLQMNSLKPEDTAMYYCAPGPNYSDYEEINWGQGTQVTVKPGGSGGSQVQVVESGGGLVQPGGSLRLSCAASGSTSLLNAMAWYRQAPGKERELVAAISSGGGTYYADSVKGRFTISTNNAKNTLYLQMNSLKPEDTAMYYCAPGPNYSDYEEINWGQGTQVTVKPGGSGGSQVQVVESGGGLVQPGGSLRLSCAASGSTSLLNAMAWYRQAPGKERELVAAISSGGGTYYADSVKGRFTISTNNAKNTLYLQMNSLKPEDTAMYYCAPGPNYSDYEEINWGQGTQVTVKP

SEQ ID NO: 52 - protein, 10 aa, synthetic construct
GSTSLLNAMA

SEQ ID NO: 53 - protein, 9 aa, synthetic construct
AISSGGGTY

SEQ ID NO: 54 - protein, 11 aa, synthetic construct
GPNYSDYEEIN

SEQ ID NO: 55 - protein, 115 aa, synthetic construct
QVQLVESGGGLVQPGGSLRLSCAASGSIASIHGMGWYRQAPGKERELVAAIAGEGSTYYADSVKGRFTISRDFAKNTLYLQMNNLKAEDTAMYYCANNDERDDYWGQGTQVTVKP

SEQ ID NO: 56 - protein, 236 aa, synthetic construct
QVQLVESGGGLVQPGGSLRLSCAASGSIASIHGMGWYRQAPGKERELVAAIAGEGSTYYADSVKGRFTISRDFAKNTLYLQMNNLKAEDTAMYYCANNDERDDYWGQGTQVTVKPGGSGGSQVQLVESGGGLVQPGGSLRLSCAASGSIASIHGMGWYRQAPGKERELVAAIAGEGSTYYADSVKGRFTISRDFAKNTLYLQMNNLKAEDTAMYYCANNDERDDYWGQGTQVTVKP

SEQ ID NO: 57 - protein, 357 aa, synthetic construct
QVQLVESGGGLVQPGGSLRLSCAASGSIASIHGMGWYRQAPGKERELVAAIAGEGSTYYADSVKGRFTISRDFAKNTLYLQMNNLKAEDTAMYYCANNDERDDYWGQGTQVTVKPGGSGGSQVQLVESGGGLVQPGGSLRLSCAASGSIASIHGMGWYRQAPGKERELVAAIAGEGSTYYADSVKGRFTISRDFAKNTLYLQMNNLKAEDTAMYYCANNDERDDYWGQGTQVTVKPGGSGGSQVQLVESGGGLVQPGGSLRLSCAASGSIASIHGMGWYRQAPGKERELVAAIAGEGSTYYADSVKGRFTISRDFAKNTLYLQMNNLKAEDTAMYYCANNDERDDYWGQGTQVTVKP

SEQ ID NO: 58 - protein, 10 aa, synthetic construct
GSIASIHGMG

SEQ ID NO: 59 - protein, 9 aa, synthetic construct
AIAGEGSTY

SEQ ID NO: 60 - protein, 7 aa, synthetic construct
NDERDDY

SEQ ID NO: 61 - protein, 114 aa, synthetic construct
QVQLVESGGGLVQPGGSLRLSCATSGIIFTSNVINWYRQAPGKQREFVAFITSITSGQSTMYADAVKGRFTVSRDNDKNIGYLQMTSLQPEDTARYVCNVGAYWGQGTQVTVKP

SEQ ID NO: 62 - protein, 234 aa, synthetic construct
QVQLVESGGGLVQPGGSLRLSCATSGIIFTSNVINWYRQAPGKQREFVAFITSITSGQSTMYADAVKGRFTVSRDNDKNIGYLQMTSLQPEDTARYVCNVGAYWGQGTQVTVKPGGSGGSQVQLVESGGGLVQPGGSLRLSCATSGIIFTSNVINWYRQAPGKQREFVAFITSITSGQSTMYADAVKGRFTVSRDNDKNIGYLQMTSLQPEDTARYVCNVGAYWGQGTQVTVKP

SEQ ID NO: 63 - protein, 10 aa, synthetic construct
GIIFTSNVIN

SEQ ID NO: 64 - protein, 12 aa, synthetic construct
FITSITSGQSTM

SEQ ID NO: 65 - protein, 227 aa, synthetic construct
DKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 66 - protein, 226 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 67 - protein, 223 aa, synthetic construct
DKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 68 - protein, 226 aa, synthetic construct
DKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 69 - protein, 6 aa, synthetic construct
GGSGGS

SEQ ID NO: 70 - protein, 4 aa, synthetic construct
GGGG

SEQ ID NO: 71 - protein, 477 aa, synthetic construct
EVQLLESGGGEVQPGGSLRLSCAASGSISIFSKYVVAWYRQAPGKEREWVAFIQSGGTANYAESAKGRFTISRDNAKNTVYLQMSSLRAEDTAVYYCNYINTDNEIGGPWGQGTLVTVRPGGSGGSEVQLLESGGGEVQPGGSLRLSCAASGSISIFSKYVVAWYRQAPGKEREWVAFIQSGGTANYAESAKGRFTISRDNAKNTVYLQMSSLRAEDTAVYYCNYINTDNEIGGPWGQGTLVTVRPGGGGDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 72 - protein, 477 aa, synthetic construct
EVQLLESGGGEVQPGGSLRLSCAASGSISIFSKYVVAWYRQAPGKEREWVAFIQSGGTANYAESAKGRFTISRDNAKNTVYLQMSSLRAEDTAVYYCNYINTDNEIGGPWGQGTLVTVRPGGSGGSEVQLLESGGGEVQPGGSLRLSCAASGSISIFSKYVVAWYRQAPGKEREWVAFIQSGGTANYAESAKGRFTISRDNAKNTVYLQMSSLRAEDTAVYYCNYINTDNEIGGPWGQGTLVTVRPGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 73 - protein, 351 aa, synthetic construct
EVQLLESGGGEVQPGGSLRLSCAASGSISIFSKYVVAWYRQAPGKEREWVAFIQSGGTANYAESAKGRFTISRDNAKNTVYLQMSSLRAEDTAVYYCNYINTDNEIGGPWGQGTLVTVRPGGGGDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 74 - protein, 9 aa, synthetic construct
GSISIFSMY

SEQ ID NO: 75 - protein, 9 aa, synthetic construct
GSISIFSDY

SEQ ID NO: 76 - protein, 9 aa, synthetic construct
GSISIFSKY

SEQ ID NO: 77 - protein, 7 aa, synthetic construct
GSTSLLN

SEQ ID NO: 78 - protein, 7 aa, synthetic construct
GSIASIH

SEQ ID NO: 79 - protein, 7 aa, synthetic construct
GIIFTSN

SEQ ID NO: 80 - protein, 5 aa, synthetic construct
QRGGT

SEQ ID NO: 81 - protein, 5 aa, synthetic construct
QSGGT

SEQ ID NO: 82 - protein, 5 aa, synthetic construct
SSGGG

SEQ ID NO: 83 - protein, 5 aa, synthetic construct
AGEGS

SEQ ID NO: 84 - protein, 8 aa, synthetic construct
TSITSGQS

SEQ ID NO: 85 - protein, 7 aa, synthetic construct
FSMYVVA

SEQ ID NO: 86 - protein, 7 aa, synthetic construct
FSDYVVA

SEQ ID NO: 87 - protein, 7 aa, synthetic construct
FSKYVVA

SEQ ID NO: 88 - protein, 5 aa, synthetic construct
LNAMA

SEQ ID NO: 89 - protein, 5 aa, synthetic construct
IHGMG

SEQ ID NO: 90 - protein, 5 aa, synthetic construct
SNVIN

SEQ ID NO: 91 - protein, 16 aa, synthetic construct
FIQRGGTANYADSAKG

SEQ ID NO: 92 - protein, 16 aa, synthetic construct
FIQRGGTANYAESAKG

SEQ ID NO: 93 - protein, 16 aa, synthetic construct
FIQRGGTANYAESAKD

SEQ ID NO: 94 - protein, 16 aa, synthetic construct
FIQSGGTANYAESAKG

SEQ ID NO: 95 - protein, 16 aa, synthetic construct
AISSGGGTYYADSVKG

SEQ ID NO: 96 - protein, 16 aa, synthetic construct
AIAGEGSTYYADSVKG

SEQ ID NO: 97 - protein, 19 aa, synthetic construct
FITSITSGQSTMYADAVKG

SEQ ID NO: 98 - protein, 8 aa, synthetic construct
IFSMYVVA

SEQ ID NO: 99 - protein, 8 aa, synthetic construct
IFSDYVVA

SEQ ID NO: 100 - protein, 8 aa, synthetic construct
IFSKYVVA

SEQ ID NO: 101 - protein, 6 aa, synthetic construct
LLNAMA

SEQ ID NO: 102 - protein, 6 aa, synthetic construct
SIHGMG

SEQ ID NO: 103 - protein, 6 aa, synthetic construct
TSNVIN

SEQ ID NO: 104 - protein, 12 aa, synthetic construct
WVAFIQRGGTAN

SEQ ID NO: 105 - protein, 12 aa, synthetic construct
WVAFIQSGGTAN

SEQ ID NO: 106 - protein, 12 aa, synthetic construct
LVAAISSGGGTY

SEQ ID NO: 107 - protein, 12 aa, synthetic construct
LVAAIAGEGSTY

SEQ ID NO: 108 - protein, 15 aa, synthetic construct
FVAFITSITSGQSTM

SEQ ID NO: 109 - protein, 11 aa, synthetic construct
NYINSDNEIGG

SEQ ID NO: 110 - protein, 11 aa, synthetic construct
NYINTDNEIGG

SEQ ID NO: 111 - protein, 12 aa, synthetic construct
APGPNYSDYEEI

SEQ ID NO: 112 - protein, 8 aa, synthetic construct
ANNDERDD

SEQ ID NO: 113 - protein, 4 aa, synthetic construct
NVGA

SEQ ID NO: 114 - protein, 10 aa, synthetic construct
GSISIFSMYV

SEQ ID NO: 115 - protein, 10 aa, synthetic construct
GSISIFSDYV

SEQ ID NO: 116 - protein, 10 aa, synthetic construct
GSISIFSKYV

SEQ ID NO: 117 - protein, 8 aa, synthetic construct
GSTSLLNA

SEQ ID NO: 118 - protein, 8 aa, synthetic construct
GSIASIHG

SEQ ID NO: 119 - protein, 8 aa, synthetic construct
GIIFTSNV

SEQ ID NO: 120 - protein, 7 aa, synthetic construct
IQRGGTA

SEQ ID NO: 121 - protein, 7 aa, synthetic construct
IQSGGTA

SEQ ID NO: 122 - protein, 7 aa, synthetic construct
ISSGGGT

SEQ ID NO: 123 - protein, 7 aa, synthetic construct
IAGEGST

SEQ ID NO: 124 - protein, 10 aa, synthetic construct
ITSITSGQST

SEQ ID NO: 125 - protein, 12 aa, synthetic construct
NYINSDNEIGGP

SEQ ID NO: 126 - protein, 12 aa, synthetic construct
NYINTDNEIGGP

SEQ ID NO: 127 - protein, 13 aa, synthetic construct
APGPNYSDYEEIN

SEQ ID NO: 128 - protein, 9 aa, synthetic construct
ANNDERDDY

SEQ ID NO: 129 - protein, 5 aa, synthetic construct
NVGAY

SEQ ID NO: 130 - protein, 120 aa, synthetic construct
EVQLLESGGGEVQPGGSLRLSCAASGSISIFSMYVVAWYRQAPGKEREWVAFIQRGGTANYAESAKGRFTISRDNAKNTVYLQMSSLRAEDTAVYYCNYIQSDNEIGGPWGQGTLVTVKP

SEQ ID NO: 131 - protein, 120 aa, synthetic construct
EVQLLESGGGEVQPGGSLRLSCAASGSISIFSMYVVAWYRQAPGKEREWVAFIQRGGTANYAESAKGRFTISRDNAKNTVYLQMSSLRAEDTAVYYCNYISSDNEIGGPWGQGTLVTVKP

SEQ ID NO: 132 - protein, 120 aa, synthetic construct
EVQLLESGGGEVQPGGSLRLSCAASGSISIFSMYVVAWYRQAPGKEREWVAFIQRGGTANYAESAKGRFTISRDNAKNTVYLQMSSLRAEDTAVYYCNYITSDNEIGGPWGQGTLVTVKP

SEQ ID NO: 133 - protein, 120 aa, synthetic construct
EVQLLESGGGEVQPGGSLRLSCAASGSISIFSYYVVAWYRQAPGKEREWVAFIQRGGTANYAESAKGRFTISRDNAKNTVYLQMSSLRAEDTAVYYCNYINSDNEIGGPWGQGTLVTVKP

SEQ ID NO: 134 - protein, 120 aa, synthetic construct
EVQLLESGGGEVQPGGSLRLSCAASGSISIFSLYVVAWYRQAPGKEREWVAFIQRGGTANYAESAKGRFTISRDNAKNTVYLQMSSLRAEDTAVYYCNYINSDNEIGGPWGQGTLVTVKP

SEQ ID NO: 135 - protein, 120 aa, synthetic construct
EVQLLESGGGEVQPGGSLRLSCAASGSISIFSIYVVAWYRQAPGKEREWVAFIQRGGTANYAESAKGRFTISRDNAKNTVYLQMSSLRAEDTAVYYCNYINSDNEIGGPWGQGTLVTVKP

SEQ ID NO: 136 - protein, 120 aa, synthetic construct
EVQLLESGGGEVQPGGSLRLSCAASGSISIFSKYVVAWYRQAPGKEREWVAFIQRGGTANYAESAKGRFTISRDNAKNTVYLQMSSLRAEDTAVYYCNYINSDNEIGGPWGQGTLVTVKP

SEQ ID NO: 137 - protein, 120 aa, synthetic construct
EVQLLESGGGEVQPGGSLRLSCAASGSISIFSSYAVAWYRQAPGKEREWVAFIQRGGTANYAESAKGRFTISRDNAKNTVYLQMSSLRAEDTAVYYCNYINSDNEIGGPWGQGTLVTVKP

SEQ ID NO: 138 - protein, 120 aa, synthetic construct
EVQLLESGGGEVQPGGSLRLSCAASGSISIFSSYAMAWYRQAPGKEREWVAFIQRGGTANYAESAKGRFTISRDNAKNTVYLQMSSLRAEDTAVYYCNYINSDNEIGGPWGQGTLVTVKP

SEQ ID NO: 139 - protein, 120 aa, synthetic construct
EVQLLESGGGEVQPGGSLRLSCAASGSISIFSSYAMSWYRQAPGKEREWVAFIQRGGTANYAESAKGRFTISRDNAKNTVYLQMSSLRAEDTAVYYCNYINSDNEIGGPWGQGTLVTVKP

SEQ ID NO: 140 - protein, 120 aa, synthetic construct
QVTLKESGGGLVRTGGSLRLSCAASGSISIFSMYVVAWYRQAPGKQREWVAFIQRGGTANYAESAKGRFTISRDNAKNTVYLQMSSLRAEDTAVYYCNYINSDNEIGGPWGQGTLVTVKP

SEQ ID NO: 141 - protein, 120 aa, synthetic construct
EVQLLESGGGLVQTGGSLRLSCAASGSISIFSMYVVAWYRQAPGKQREWVAFIQRGGTANYAESAKGRFTISRDNAKNTVYLQMSSLRAEDTAVYYCNYINSDNEIGGPWGQGTLVTVKP

SEQ ID NO: 142 - protein, 120 aa, synthetic construct
EVQLLESGGGLVRPGGSLRLSCAASGSISIFSMYVVAWYRQAPGKQREWVAFIQRGGTANYAESAKGRFTISRDNAKNTVYLQMSSLRAEDTAVYYCNYINSDNEIGGPWGQGTLVTVKP

SEQ ID NO: 143 - protein, 120 aa, synthetic construct
EVQLLESGGGEVQTGGSLRLSCAASGSISIFSMYVVAWYRQAPGKQREWVAFIQRGGTANYAESAKGRFTISRDNAKNTVYLQMSSLRAEDTAVYYCNYINSDNEIGGPWGQGTLVTVKP

SEQ ID NO: 144 - protein, 120 aa, synthetic construct
EVQLLESGGGEVRPGGSLRLSCAASGSISIFSMYVVAWYRQAPGKQREWVAFIQRGGTANYAESAKGRFTISRDNAKNTVYLQMSSLRAEDTAVYYCNYINSDNEIGGPWGQGTLVTVKP

SEQ ID NO: 145 - protein, 120 aa, synthetic construct
EVQLLESGGGLVRTGGSLRLSCAASGSISIFSMYVVAWYRQAPGKQREWVAFIQRGGTANYAESAKGRFTISRDNAKNTVYLQMSSLRAEDTAVYYCNYINSDNEIGGPWGQGTLVTVKP

SEQ ID NO: 146 - protein, 120 aa, synthetic construct; identical to SEQ ID NO: 145

SEQ ID NO: 147 - protein, 120 aa, synthetic construct
EVQLLESGGGEVQTGGSLRLSCAASGSISIFSMYVVAWYRQAPGKQREWVAFIQRGGTANYAESAKGRFTISRDNAKNTVYLQMSSLRPEDTAVYYCNYINSDNEIGGPWGQGTLVTVKP

SEQ ID NO: 148 - protein, 120 aa, synthetic construct
EVQLLESGGGEVQTGGSLRLSCAASGSISIFSMYVVAWYRQAPGKQREWVAFIQRGGTANYAESAKGRFTISRDNAKNTVYLQMSSLRADDTAVYYCNYINSDNEIGGPWGQGTLVTVKP

SEQ ID NO: 149 - protein, 9 aa, synthetic construct
  VARIANT 8: note = M, K, D, Y, L, or I
GSISIFSXY

SEQ ID NO: 150 - protein, 5 aa, synthetic construct
  VARIANT 2: note = R or S
QXGGT

SEQ ID NO: 151 - protein, 10 aa, synthetic construct
  VARIANT 2: note = N, Q, S, or T
  VARIANT 3: note = S or T
IXXDNEIGGP

SEQ ID NO: 152 - protein, 7 aa, synthetic construct
  VARIANT 3: note = M, K, D, Y, L, or I
FSXYVVA

SEQ ID NO: 153 - protein, 16 aa, synthetic construct
  VARIANT 4: note = R or S
  VARIANT 16: note = G or D
FIQXGGTANYAESAKX

SEQ ID NO: 154 - protein, 10 aa, synthetic construct
  VARIANT 2: note = N, Q, S or T
  VARIANT 3: note = S or T
IXXDNEIGGP

SEQ ID NO: 155 - protein, 8 aa, synthetic construct
  VARIANT 4: note = M, K, D, Y, L or I
IFSXYVVA

SEQ ID NO: 156 - protein, 12 aa, synthetic construct
  VARIANT 3: note = A or S
  VARIANT 7: note = R or S
WVXFIQXGGTAN

SEQ ID NO: 157 - protein, 11 aa, synthetic construct
  VARIANT 4: note = N, Q, S or T
  VARIANT 5: note = S or T
NYIXXDNEIGG

SEQ ID NO: 158 - protein, 10 aa, synthetic construct
  VARIANT 8: note = M, K, D, Y, L or I
GSISIFSXYV

SEQ ID NO: 159 - protein, 7 aa, synthetic construct
  VARIANT 3: note = R or S
IQXGGTA

SEQ ID NO: 160 - protein, 12 aa, synthetic construct
  VARIANT 4: note = N, Q, S, or T
  VARIANT 5: note = S or T
NYIXXDNEIGGP

SEQ ID NO: 161 - protein, 120 aa, synthetic construct
EVQLLESGGGEVQPGGSLRLSCAASGSISIFSMYVVAWYRQAPGKEREWVAFIQRGGTANYAESAKGRFTISRDNAKNTVYLQMSSLRAEDTAVYYCNYINSDNEIGGPWGQGTLVTVKP

SEQ ID NO: 162 - protein, 120 aa, synthetic construct
EVQLLESGGGEVQPGGSLRLSCAASGSISIFSMYVVAWYRQAPGKQREWVSAIQRGGTANYAESAKGRFTISRDNAKNTVYLQMSSLRAEDTAVYYCNYINSDNEIGGPWGQGTLVTVKP

SEQ ID NO: 163 - protein, 120 aa, synthetic construct
EVQLLESGGGEVQPGGSLRLSCAASGSISIFSMYVVAWYRQAPGKQREWVSFIQRGGTANYAESAKGRFTISRDNAKNTVYLQMSSLRAEDTAVYYCNYINSDNEIGGPWGQGTLVTVKP

SEQ ID NO: 164 - protein, 120 aa, synthetic construct
QVTLKESGGGEVQPGGSLRLSCAASGSISIFSMYVVAWYRQAPGKQREWVAFIQRGGTANYAESAKGRFTISRDNAKNTVYLQMSSLRAEDTAVYYCNYINSDNEIGGPWGQGTLVTVKP

SEQ ID NO: 165 - protein, 354 aa, synthetic construct
QVQLVESGGGLVQPGGSLRLSCATSGIIFTSNVINWYRQAPGKQREFVAFITSITSGQSTMYADAVKGRFTVSRDNDKNIGYLQMTSLQPEDTARYVCNVGAYWGQGTQVTVKPGGSGGSQVQLVESGGGLVQPGGSLRLSCATSGIIFTSNVINWYRQAPGKQREFVAFITSITSGQSTMYADAVKGRFTVSRDNDKNIGYLQMTSLQPEDTARYVCNVGAYWGQGTQVTVKPGGSGGSQVQLVESGGGLVQPGGSLRLSCATSGIIFTSNVINWYRQAPGKQREFVAFITSITSGQSTMYADAVKGRFTVSRDNDKNIGYLQMTSLQPEDTARYVCNVGAYWGQGTQVTVKP

(19) United States
(12) Patent Application Publication
(10) Pub. No.: US 2023/0406925 A1
(43) Pub. Date: Dec. 21, 2023

(54) SIGLEC-8 BINDING PROTEINS AND USES THEREOF

(71) Applicants: Phylaxis Bioscience, LLC, Denver, CO (US); Inhibrx, Inc., La Jolla, CA (US)

(72) Inventors: Tony Dung-Ling Yao, Denver, CO (US); Andrew M. Eckles, La Jolla, CA (US); Jacob J. Gano, Encinitas, CA (US); Bryan Becklund, San Diego, CA (US); Brendan P. Eckelman, Encinitas, CA (US)

(21) Appl. No.: 18/361,184

(22) Filed: Jul. 28, 2023

Related U.S. Application Data
(63) Continuation of application No. PCT/US23/66086, filed on Apr. 21, 2023.
Application No. PCT/US23/66086 is a division of application No. 63/333,952, filed on Apr. 22, 2022.

(51) Int. Cl. C07K 16/28; C12N 15/63; A61P 37/06

(52) U.S. Cl. CPC C07K 16/2803; C12N 15/63; A61P 37/06; C07K 2317/565; C07K 2317/24; C07K 2317/52

(57) ABSTRACT

Provided herein are Siglec-8 binding proteins, and methods of using Siglec-8 binding proteins to modulate the biological activity of Siglec-8.

### CROSS-REFERENCE TO RELATED APPLICATIONS

This application claims the benefit of priority of U.S. Provisional Application No. 63/333,952, filed Apr. 22, 2022; the contents of which are incorporated by reference in their entirety for all purposes.

### INCORPORATION BY REFERENCE OF SEQUENCE LISTING

This application incorporates by reference a Sequence Listing submitted with this application in electronic format entitled PXS-002WO_SL.xml, created Apr. 19, 2023, which is 163,709 bytes in size.

### FIELD

The present invention relates to Siglec-8 binding proteins, and methods of using such proteins to modulate the biological activity of Siglec-8. Such methods include, but are not limited to, methods of treating an eosinophilic disorder or a mast cell disorder.

### BACKGROUND

Mast cells are tissue-resident cells that regulate acute and chronic tissue inflammation. Aberrant accumulation and activation of mast cells have been known to play a role in mediating allergic diseases such as eosinophilic asthma, atopic dermatitis, and eosinophilic gastrointestinal diseases (Schanin et al. Mucosal Immunology 2021, 14, 366-376). Sialic acid-binding immunoglobulin-like lectin 8 (Siglec-8) is an inhibitory cell surface receptor expressed selectively on these mast cells as well as mature eosinophils and basophils, which has hence been gaining attention as a target to treat allergic and inflammatory diseases. Crosslinking Siglec-8 has shown to induce eosinophil death, in which the process is dependent on release of reactive oxygen species (ROS) and activation of beta2-integrin (Legrand et al. J Allergy Clin Immunol 2009, 143, 2227-2237). The selective expression of Siglec-8 renders it a lucrative target which may reduce side effects and increase drug safety.

### SUMMARY

Provided herein are Siglec-8 binding VHH domains, polypeptides, and proteins, and methods of using the Siglec-8 binding VHH domains, polypeptides, and proteins to medical treatments. For example, the VHH domains, polypeptides, and proteins disclosed herein are useful for treating eosinophilic disorders, including Eosinophilic Cystitis, Eosinophilic Fasciitis, Eosinophilic Gastrointestinal Disorders, Eosinophilic Gastritis (EoG), Eosinophilic Enteritis, Churg-Strauss syndrome, hypereosinophilic syndrome. They are also useful for treating mast cell disorders, including but not limited to Mast Cell Leukemia, Aggressive Systemic Mastocytosis, and Indolent Mastocytosis. In addition, they are useful for treating general allergic conditions such as those induced by food allergies, environmental allergies, venom allergies, and/or companion animal allergies. In some embodiments, a Siglec-8 binding polypeptide comprises at least one VHH domain. Some embodiments are provided below.

In one aspect, the present disclosure provides an VHH domain that binds Siglec-8 (e.g., human Siglec-8), comprising complementarity determining region 1 (CDR1), complementarity determining region 2 (CDR2), and complementarity determining region 3 (CDR3) sequences of a VHH that comprises an amino acid sequence selected from the group consisting of SEQ ID NO: 26, 5, 11, 16, 21, 31, 36, 130-138, 140-148, 161, 163, and 164.

In another aspect, the present disclosure provides a VHH domain that binds Siglec-8 (e.g., human Siglec-8), comprising CDR1, CDR2, and CDR3 sequences set forth in: (a) SEQ ID NOs: 46, 47, and 48, respectively, as defined according to the AbM numbering system; (b) SEQ ID NOs: 149, 150, and 151, respectively, as defined according to the Chothia numbering system; (c) SEQ ID NOs: 152, 153, and 154, respectively, as defined according to the Kabat numbering system; (d) SEQ ID NOs: 155, 156, and 157, respectively, as defined according to the Contact numbering system; or (e) SEQ ID NOs: 158, 159, and 160, respectively, as defined according to the IMGT numbering system. As defined according to the AbM numbering system, the CDR1 can comprise the amino acid sequence of SEQ ID NO: 18, 2, or 13; the CDR2 can comprise the amino acid sequence of SEQ ID NO: 29 or 3; and the CDR3 can comprise the amino acid sequence of SEQ ID NO: 8. As defined according to the Chothia numbering system, the CDR1 can comprise the amino acid sequence of SEQ ID NO: 74, 75, or 76; the CDR2 can comprise the amino acid sequence of SEQ ID NO: 80 or 81; and the CDR3 can comprise the amino acid sequence of SEQ ID NO: 8. As defined according to the Kabat numbering system, the CDR1 can comprise the amino acid sequence of SEQ ID NO: 85, 86, or 87; the CDR2 can comprise the amino acid sequence of SEQ ID NO: 92, 93, or 94; and the CDR3 can comprise the amino acid sequence of SEQ ID NO: 8. As defined according to the Contact numbering system, the CDR1 can comprise the amino acid sequence of SEQ ID NO: 98, 99, or 100; the CDR2 can comprise the amino acid sequence of SEQ ID NO: 104 or 105; and the CDR3 can comprise the amino acid sequence of SEQ ID NO: 110. As defined according to the IMGT numbering system, the CDR1 can comprise the amino acid sequence of SEQ ID NO: 114, 115, or 116; the CDR2 can comprise the amino acid sequence of SEQ ID NO: 120 or 121; and the CDR3 can comprise the amino acid sequence of SEQ ID NO: 126.

In certain embodiments of any of the aspects above, the CDR1, CDR2, and CDR3 sequences are set forth in SEQ ID NOs: 18, 29, and 8; 2, 3, and 8; 13, 3, and 8; or 18, 3, and 8, respectively, as defined according to the AbM numbering system; in SEQ ID NOs: 74, 80, and 8; 75, 80, and 8; 76, 80, and 8; or 76, 81, and 8, respectively, as defined according to the Chothia numbering system; in SEQ ID NOs: 85, 92, and 8; 86, 92, and 8; 87, 93, and 8; 87, 92, and 8; or 87, 94, and 8, respectively, as defined according to the Kabat numbering system; in SEQ ID NOs: 98, 104, and 110; 99, 104, and 110; 100, 104, and 110; or 100, 105, and 110, respectively, as defined according to the Contact numbering system; or in SEQ ID NOs: 114, 120, and 126; 115, 120, and 126; 116, 120, and 126; or 116, 121, and 126, respectively, as defined according to the IMGT numbering system.

In certain embodiments, the VHH domain comprises an amino acid sequence that is at least 80%, at least 85%, at least 90%, at least 95%, at least 96%, at least 97%, at least 98%, at least 99%, or 100% identical to an amino acid sequence selected from the group consisting of SEQ ID NO: 26, 5, 11, 16, 21, 31, 36, 130-138, 140-148, 161, 163, and 164. In certain embodiments, the VHH domain is humanized.

The present disclosure also provides an VHH domain that binds Siglec-8 (e.g., human Siglec-8), comprising CDR1, CDR2, and CDR3 sequences of a VHH that comprises the amino acid sequence of SEQ ID NO: 1. In certain embodiments, the CDR1, CDR2, and CDR3 comprise the amino acid sequences set forth in: (a) SEQ ID NOs: 2, 3, and 4, respectively, as defined according to the AbM numbering system; (b) SEQ ID NOs: 74, 80, and 4, respectively, as defined according to the Chothia numbering system; (c) SEQ ID NOs: 85, 91, and 4, respectively, as defined according to the Kabat numbering system; (d) SEQ ID NOs: 98, 104, and 109, respectively, as defined according to the Contact numbering system; or (e) SEQ ID NOs: 114, 120, and 125, respectively, as defined according to the IMGT numbering system. In certain embodiments, the VHH domain comprises an amino acid sequence that is at least 80%, at least 85%, at least 90%, at least 95%, at least 96%, at least 97%, at least 98%, at least 99%, or 100% identical to the amino acid sequence of SEQ ID NO: 1.

The present disclosure also provides an VHH domain that binds Siglec-8 (e.g., human Siglec-8), comprising CDR1, CDR2, and CDR3 sequences of a VHH that comprises the amino acid sequence of SEQ ID NO: 49. In certain embodiments, the CDR1, CDR2, and CDR3 comprise the amino acid sequences set forth in: (a) SEQ ID NOs: 52, 53, 54, respectively, as defined according to the AbM numbering system; (b) SEQ ID NOs: 77, 82, 54, respectively, as defined according to the Chothia numbering system; (c) SEQ ID NOs: 88, 95, 54, respectively, as defined according to the Kabat numbering system; (d) SEQ ID NOs: 101, 106, 111, respectively, as defined according to the Contact numbering system; or (e) SEQ ID NOs: 117, 122, 127, respectively, as defined according to the IMGT numbering system. In certain embodiments, the VHH domain comprises an amino acid sequence that is at least 80%, at least 85%, at least 90%, at least 95%, at least 96%, at least 97%, at least 98%, at least 99%, or 100% identical to the amino acid sequence of SEQ ID NO: 49.

The present disclosure also provides an VHH domain that binds Siglec-8 (e.g., human Siglec-8), comprising CDR1, CDR2, and CDR3 sequences of a VHH that comprises the amino acid sequence of SEQ ID NO: 55. In certain embodiments, the CDR1, CDR2, and CDR3 comprise the amino acid sequences set forth in: (a) SEQ ID NOs: 58, 59, 60, respectively, as defined according to the AbM numbering system; (b) SEQ ID NOs: 78, 83, 60, respectively, as defined according to the Chothia numbering system; (c) SEQ ID NOs: 89, 96, 60, respectively, as defined according to the Kabat numbering system; (d) SEQ ID NOs: 102, 107, 112, respectively, as defined according to the Contact numbering system; or (e) SEQ ID NOs: 118, 123, 128, respectively, as defined according to the IMGT numbering system. In certain embodiments, the VHH domain comprises an amino acid sequence that is at least 80%, at least 85%, at least 90%, at least 95%, at least 96%, at least 97%, at least 98%, at least 99%, or 100% identical to the amino acid sequence of SEQ ID NO: 55.

The present disclosure also provides an VHH domain that binds Siglec-8 (e.g., human Siglec-8), comprising CDR1, CDR2, and CDR3 sequences of a VHH that comprises the amino acid sequence of SEQ ID NO: 61. In certain embodiments, the CDR1, CDR2, and CDR3 comprise: (a) the amino acid sequence of SEQ ID NO: 63, the amino acid sequence of SEQ ID NO: 64, and the amino acid sequence of Gly-Ala-Tyr (GAY), respectively, as defined according to the AbM numbering system; (b) the amino acid sequence of SEQ ID NO: 79, the amino acid sequence of SEQ ID NO: 84, and the amino acid sequence of Gly-Ala-Tyr (GAY), respectively, as defined according to the Chothia numbering system; (c) the amino acid sequence of SEQ ID NO: 90, the amino acid sequence of SEQ ID NO: 97, and the amino acid sequence of Gly-Ala-Tyr (GAY), respectively, as defined according to the Kabat numbering system; (d) the amino acid sequences of SEQ ID NOs: 103, 108, 113, respectively, as defined according to the Contact numbering system; or (e) the amino acid sequences of SEQ ID NOs: 119, 124, 129, respectively, as defined according to the IMGT numbering system. In certain embodiments, the VHH domain comprises an amino acid sequence that is at least 80%, at least 85%, at least 90%, at least 95%, at least 96%, at least 97%, at least 98%, at least 99%, or 100% identical to the amino acid sequence of SEQ ID NO: 61.

In another aspect, the present disclosure provides a polypeptide comprising a VHH domain disclosed herein. The polypeptide can comprise at least two of such VHH domains. In certain embodiments, the two VHH domains are operably linked to each other via a peptide linker, e.g., a peptide linker comprising or consisting of the amino acid sequence of SEQ ID NO: 69. In certain embodiments, the VHH domains in the polypeptide comprise the same CDR1, CDR2, and CDR3 amino acid sequences. In certain embodiments, the VHH domains in the polypeptide comprise the same VHH amino acid sequence.

In certain embodiments, the polypeptide comprises an amino acid sequence that is at least 80%, at least 85%, at least 90%, at least 95%, at least 97%, or 100% identical to the amino acid sequence of SEQ ID NO: 9, 10, 12, 17, 22, 27, 32, 37, 50, 51, 56, 57, 62, or 63.

The polypeptide disclosed herein can further comprise a multimerization domain.

In another aspect, the present disclosure provides a polypeptide comprising at least two (e.g., two, or three) antigen-binding domains that bind Siglec-8 (e.g., human Siglec-8) and a multimerization domain.

In another aspect, the present disclosure provides a polypeptide that binds Siglec-8 (e.g., human Siglec-8), comprising at least three antigen-binding domains that bind Siglec-8 (e.g., human Siglec-8).

In certain embodiments of the two aspects above, each of the antigen-binding domains is a VHH domain. In other embodiments of these aspects, each of the antigen-binding domains comprises a heavy chain variable region and a light chain variable region, e.g., in the format of a Fab or scFv.

In certain embodiments of any of the applicable aspects above, the multimerization domain is a dimerization domain, for example, an antibody Fc region, such as a human IgG1 Fc region. The antibody Fc region can comprise the amino acid sequence of SEQ ID NO: 44, 45, 66, 67, or 68.

The antibody Fc region can be operably linked to at least one of the antigen-binding domains that bind Siglec-8 via an amino acid linker, e.g., an amino acid linker comprising or consisting of the amino acid sequence of SEQ ID NO: 70.

The polypeptide can take various structural formats. In certain embodiments, the polypeptide comprises the structure VHH-VHH-Fc. Such polypeptide can comprise an amino acid sequence at least 80%, at least 85%, at least 90%, at least 95%, at least 96%, at least 97%, at least 98%, at least 99%, or 100% identical to the amino acid sequence of SEQ ID NO: 71. In certain embodiments, the polypeptide comprises the structure VHH-Fc-VHH. In certain embodiments, the polypeptide comprises the structure VHH-VHH-VHH-Fc or VHH-VHH-Fc-VHH.

The present disclosure also provides a protein comprising two or more polypeptides disclosed herein multimerized under physiological conditions via the multimerization domain. For example, the two or more polypeptides can form a homomultimer. Where the multimerization domain is a dimerization domain, the protein can comprise two polypeptides disclosed herein dimerized under physiological conditions via the dimerization domain. For example, the two polypeptides can form a homodimer.

Where the VHH, polypeptide, or protein disclosed herein binds human Siglec-8, the amino acid sequence of the human Siglec-8 can be set forth in SEQ ID NO: 43.

In certain embodiments, the polypeptide or protein disclosed herein mediates eosinophil killing in the presence of IL-5. The eosinophil killing can be determined in an in vitro assay. It is contemplated that where the protein comprises at least two VHH domains that bind Siglec-8 in each polypeptide, a homodimer protein can mediate eosinophil killing in the presence of IL-5 with a lower EC50 than a homodimer of polypeptides each having the structure of VHH-Fc and comprising only one of the VHH domains that binds Siglec-8.

In certain embodiments, the polypeptide or protein mediates eosinophil killing in the absence of IL-5. In certain embodiments, the polypeptide mediates eosinophil killing through antibody-dependent cell-mediated cytotoxicity (ADCC).

The present disclosure also provides a pharmaceutical composition comprising a polypeptide or protein disclosed herein, and a pharmaceutically acceptable carrier.

In addition, the present disclosure provides an isolated nucleic acid that encodes a polypeptide disclosed herein, a vector comprising the nucleic acid, a host cell comprising the nucleic acid or the vector, and a host cell that expresses the polypeptide or protein.

The present disclosure also provides a method of producing a polypeptide or protein, the method comprising incubating the host cell under conditions for expression of the polypeptide or protein. The method can further comprise isolating the polypeptide or protein.

Another aspect of the present disclosure provides a method of treating an eosinophilic disorder or a mast cell disorder, the method comprising administering to a subject in need thereof a therapeutically effective amount of a polypeptide, protein, or pharmaceutical composition disclosed herein. The eosinophilic disorder can be eosinophilic cystitis, eosinophilic fasciitis, an eosinophilic gastrointestinal disorder, eosinophilic gastritis (EoG), eosinophilic enteritis, Churg-Strauss syndrome, hypereosinophilic syndrome, eosinophilic leukemias (e.g., chronic eosinophilic leukemia), asthma with an eosinophilic phenotype, allergic bronchopulmonary aspergillosis (ABPA), chronic rhinosinusitis with nasal polyps (CRSwNP), or eosinophilic granulomatosis with polyangiitis (EGPA). The mast cell disorder can be Systemic Mastocytosis, hereditary alpha tryptasemia (HAT), or mast cell activation syndrome (MCAS). The Systemic Mastocytosis (SM) can be advanced SM (e.g., Mast Cell Leukemia, aggressive SM, or SM with associated hematologic neoplasm (SM-AHN)) or non-advanced SM (e.g., indolent SM).

The present disclosure also provides a method of treating an inflammatory disease or condition, the method comprising administering to a subject in need thereof a therapeutically effective amount of a polypeptide, protein, or pharmaceutical composition disclosed herein.

In addition, the present disclosure provides a method of treating or preventing an allergic condition, the method comprising administering to a subject in need thereof a therapeutically effective amount of a polypeptide, protein, or pharmaceutical composition disclosed herein. The allergic condition can be a food allergy, an environmental allergy, a companion animal allergy, and/or a venom allergy.

In addition, the present disclosure provides a method of depleting eosinophils in a subject, the method comprising administering to the subject a therapeutically effective amount of a polypeptide, protein, or pharmaceutical composition disclosed herein.

Further, the present disclosure provides a method of killing an eosinophil, the method comprising contacting the eosinophil with a natural killer (NK) cell in the presence of a polypeptide, protein, or pharmaceutical composition disclosed herein. The present disclosure also provides a method of killing an eosinophil, the method comprising contacting the eosinophil with a macrophage in the presence of a polypeptide, protein, or pharmaceutical composition disclosed herein.

### BRIEF DESCRIPTION OF THE FIGURES

FIGS. 1A-1D show the mean fluorescence intensity of parental anti-human Siglec8-B12 (cx8982; FIG. 1A), Siglec8-D1 (cx8983; FIG. 1B), Siglec8-E2 (cx8984; FIG. 1C), or Siglec8-G2 (cx10382; FIG. 1D) binding to either non-transfected (UT-CHO) or human Siglec-8 expressing CHO cells as determined by binding of an A647-conjugated anti-human Fcγ specific secondary antibody using flow cytometry.

FIGS. 2A-2D show the mean fluorescence intensity of binding to human Siglec-8 on eosinophils (FIGS. 2A-2C) or the median fluorescence intensity of binding to human Siglec-8 transfected CHO cells (FIG. 2D) by bivalent, tetravalent, and hexavalent anti-Siglec8-B12 antibodies (cx8982, cx8467, cx8466; FIG. 2A), anti-Siglec8-D1 antibodies (cx8983, cx8469, cx8523; FIG. 2B), anti-Siglec8-E2 antibodies (cx8984, cx8463, cx8522; FIG. 2C), or anti-Siglec8-G2v14 antibodies (cx11422, cx11580, cx11579; FIG. 2D) as determined by binding of an A647-conjugated anti-human Fcγ specific secondary antibody using flow cytometry.

FIGS. 3A-3E show the total red object integrated intensity (RCU×μm2/image) of the dead cell marker cytotox red incorporated in purified human eosinophils that were treated with titrating amounts of bivalent, tetravalent, or hexavalent anti-Siglec8-B12 antibodies (cx8982, cx8467, cx8466; FIG. 3A), anti-Siglec8-D1 antibodies (cx8983, cx8469, cx8523; FIG. 3B), anti-Siglec8-E2 antibodies (cx8984, cx8463, cx8522; FIG. 3C), in the presence of IL-5 after 24 hours, or anti-Siglec8-G2v14 (cx11422, cx11580, cx11579) agonist antibodies after 18 hours in the presence of IL-5 (FIG. 3D) or absence of IL-5 (FIG. 3E).

FIGS. 4A-4C show the median fluorescence intensity of binding of tetravalent Siglec-8 agonist antibodies comprising hzG2v52 (cx11913) or hzG2v53 (cx11914) and comparator analog 2E2 Afuc to freshly isolated human eosinophils (FIG. 4A), or the mean fluorescence intensity of tetravalent Siglec-8 agonist antibodies comprising hzG2v52 (cx11913), hzG2v53 (cx11914), or hzG2v14 (cx11769) binding to human Siglec-2, 3, 6, 7, 8, 9, 12, and 15 transfected CHO cells (FIGS. 4B-4C), all as determined by binding of an A647-conjugated anti-human Fcγ specific secondary antibody using flow cytometry.

FIGS. 5A-5H show the median fluorescence intensity of parental anti-human Siglec8-G2 (1mG2) and humanized variants thereof formatted a monomeric VHH-hIgG1 fusions binding to either human Siglec-8 expressing CH0293 cells (FIGS. 5A-5D) or non-transfected 293 cells (FIGS. 5E-5H) as determined by binding of an A647-conjugated anti-human Fcγ specific secondary antibody using flow cytometry.

FIGS. 6A-6B show the direct (FIG. 6A) and ADCC-mediated (FIG. 6B) killing activity of multivalent Siglec-8 agonist antibodies comprising hzG2v53 having engineered Fc regions (cx12562 and/or cx11914) or a tetravalent having a wild type Fc region (cx12532) and the comparator analog 2E2 Afuc. FIG. 6A shows the total red object integrated intensity (RCU×μm2/image) of the dead cell marker cytotox red incorporated in purified human eosinophils in the presence of IL-5. FIG. 6B shows the percent of apoptotic, apotracker green positive eosinophils killed through NK cell-mediated ADCC in the absence of IL-5.

FIG. 7 depicts exemplary formats of multivalent Siglec-8 targeting polypeptides compared to bivalent Siglec-8 targeting polypeptides.

### DETAILED DESCRIPTION

Embodiments provided herein relate to Siglec-8 binding polypeptides and their use in various methods of treating Siglec-8-related diseases or disorders.

### Definitions and Various Embodiments

The section headings used herein are for organizational purposes only and are not to be construed as limiting the subject matter described.

All references cited herein, including patent applications, patent publications, and GenBank accession numbers are herein incorporated by reference, as if each individual reference were specifically and individually indicated to be incorporated by reference in its entirety.

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3rd. edition (2001) Cold Spring Harbor Laboratory Press, Cold Spring Harbor, N.Y. CURRENT PROTOCOLS IN MOLECULAR BIOLOGY (F. M. Ausubel, et al. eds., (2003)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J.B. Lippincott Company, 1993); and updated versions thereof.

Unless otherwise defined, scientific and technical terms used in connection with the present disclosure shall have the meanings that are commonly understood by those of ordinary skill in the art. Further, unless otherwise required by context or expressly indicated, singular terms shall include pluralities and plural terms shall include the singular. For any conflict in definitions between various sources or references, the definition provided herein will control.

In general, the numbering of the residues in an immunoglobulin heavy chain is that of the EU index as in Kabat et al., Sequences of Proteins of Immunological Interest, 5th Ed. Public Health Service, National Institutes of Health, Bethesda, Md. (1991). The “EU index as in Kabat” refers to the residue numbering of the human IgG1 EU antibody.

It is understood that embodiments of the invention described herein include “comprising,” “consisting,” and/or “consisting essentially of” embodiments. As used herein, the singular form “a”, “an”, and “the” includes plural references unless indicated otherwise. Use of the term “or” herein is not meant to imply that alternatives are mutually exclusive.

In this application, the use of “or” means “and/or” unless expressly stated or understood by one skilled in the art. In the context of a multiple dependent claim, the use of “or” refers back to more than one preceding independent or dependent claim.

The phrase “reference sample”, “reference cell”, or “reference tissue”, denote a sample with at least one known characteristic that can be used as a comparison to a sample with at least one unknown characteristic. In some embodiments, a reference sample can be used as a positive or negative indicator. A reference sample can be used to establish a level of protein and/or mRNA that is present in, for example, healthy tissue, in contrast to a level of protein and/or mRNA present in the sample with unknown characteristics. In some embodiments, the reference sample comes from the same subject, but is from a different part of the subject than that being tested. In some embodiments, the reference sample is from a tissue area surrounding or adjacent to the cancer. In some embodiments, the reference sample is not from the subject being tested, but is a sample from a subject known to have, or not to have, a disorder in question (for example, a Siglec-8 related disorder). In some embodiments, the reference sample is from the same subject, but from a point in time before the subject developed the disorder. In some embodiments, the reference sample is from the same or a different subject. When a negative reference sample is used for comparison, the level of expression or amount of the molecule in question in the negative reference sample will indicate a level at which one of skill in the art will appreciate, given the present disclosure, that there is no and/or a low level of the molecule. When a positive reference sample is used for comparison, the level of expression or amount of the molecule in question in the positive reference sample will indicate a level at which one of skill in the art will appreciate, given the present disclosure, that there is a level of the molecule.

The terms “benefit”, “clinical benefit”, “responsiveness”, and “therapeutic responsiveness” as used herein in the context of benefiting from or responding to administration of a therapeutic agent, can be measured by assessing various endpoints, e.g., inhibition, to some extent, of disease progression, including slowing down and complete arrest; reduction in the number of disease episodes and/or symptoms; reduction in lesion size; inhibition (that is, reduction, slowing down or complete stopping) of disease cell infiltration into adjacent peripheral organs and/or tissues; inhibition (that is, reduction, slowing down or complete stopping) of disease spread; relief, to some extent, of one or more symptoms associated with the disorder; increase in the length of disease-free presentation following treatment, for example, progression-free survival; increased overall survival; higher response rate; and/or decreased mortality at a given point of time following treatment. A subject or cancer that is “non-responsive”or “fails to respond” is one that has failed to meet the above noted qualifications to be “responsive”.

The terms “nucleic acid molecule”, “nucleic acid” and “polynucleotide” may be used interchangeably, and refer to a polymer of nucleotides. Such polymers of nucleotides may contain natural and/or non-natural nucleotides, and include, but are not limited to, DNA, RNA, and PNA. “Nucleic acid sequence” refers to the linear sequence of nucleotides comprised in the nucleic acid molecule or polynucleotide.

The term “polypeptide” refers to a polymer of amino acid residues, and are not limited to a minimum length. Such polymers of amino acid residues may contain natural or non-natural amino acid residues, and include, but are not limited to, peptides, oligopeptides, and full-length forms and fragments of natural polypeptides. The terms also include post-expression modifications of the polypeptide, for example, glycosylation, sialylation, acetylation, phosphorylation, and the like. Furthermore, for purposes of the present disclosure, a “polypeptide” can include modifications, such as deletions, additions, and substitutions (generally conservative in nature), to the native sequence, as long as the it maintains the desired activity. These modifications may be deliberate, as through site-directed mutagenesis, or may be accidental, such as through mutations of hosts which produce the proteins or errors due to PCR amplification.

The term “protein” refers to a macromolecule comprising one or more polypeptides. For example, a protein can be a multimer (e.g., dimer, trimer, or tetramer) formed by a plurality of the same or different polypeptides.

“Siglec-8” as used herein refers to any native, mature Siglec-8 that results from processing of a Siglec-8 precursor in a cell. The term includes Siglec-8 from any mammalian source, including primates (e.g., humans and cynomolgus or rhesus monkeys) and rodents (e.g., mice and rats), unless otherwise indicated. A nonlimiting exemplary human Siglec-8 amino acid sequence is shown, e.g., in UniProtKB/Swiss-Prot: Q9NYZ4.2. See SEQ ID NO. 42. A nonlimiting exemplary mature human Siglec-8 amino acid sequence is shown, e.g., in SEQ ID NO: 43.

The term “specifically binds” to an antigen or epitope is a term that is well understood in the art, and methods to determine such specific binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. A single-domain antibody (sdAb) or VHH-containing polypeptide “specifically binds” or “preferentially binds” to a target if it binds with greater affinity, avidity, more readily, and/or with greater duration than it binds to other substances. For example, a sdAb or VHH-containing polypeptide that specifically or preferentially binds to a Siglec-8 epitope is a sdAb or VHH-containing polypeptide that binds this epitope with greater affinity, avidity, more readily, and/or with greater duration than it binds to other Siglec-8 epitopes or non-Siglec-8 epitopes. It is also understood by reading this definition that; for example, a sdAb or VHH-containing polypeptide that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding. “Specificity” refers to the ability of a binding protein to selectively bind an antigen.

The terms “inhibition” or “inhibit” refer to a decrease, cessation, or suppression of any phenotypic characteristic or to the decrease, cessation, or suppression in the incidence, degree, or likelihood of that characteristic. To “reduce” or “inhibit” is to decrease, reduce, suppress, or arrest an activity, function, amount, abundance, or rate of increase as compared to a reference. In some embodiments, by “reduce” or “inhibit” is meant the ability to cause an overall decrease of 10% or greater. In some embodiments, by “reduce” or “inhibit” is meant the ability to cause an overall decrease of 50% or greater. In some embodiments, by “reduce” or “inhibit” is meant the ability to cause an overall decrease of 75%, 85%, 90%, 95%, or greater. In some embodiments, the amount noted above is inhibited or decreased over a period of time, relative to a control over the same period of time.

The terms “agonize” or “activate” refer to an increase, activation, or induction of any phenotypic characteristic or to the increase, activation, or induction in the incidence of likelihood of that characteristic. To “agonize” or “activate” is to increase, activate, or induce an activity, function, amount, abundance, or rate of increase as compared to a reference. In some embodiments, by “agonize” or “activate” is meant the ability to cause an overall increase of 10% or greater. In some embodiments, by “agonize” or “activate” is meant an ability to cause an overall increase of 50% or greater. In some embodiments, by “agonize” or “activate” is meant the ability to cause an overall increase of 75%, 85%, 90%, 95%, or greater. In some embodiments, the amount noted above is increased over a period of time, relative to a control over the same period of time. As used herein, the term “agonize” with regard to the activity of Siglec-8 refers to an increase in an activity of Siglec-8, which is an inhibitory receptor on mast cells and eosinophils that mediates cell death of eosinophils in the presence of IL-5 and negatively regulates mast cell activation. Accordingly, in some embodiments, an agonist of Siglec-8 increases eosinophil cell death in the presence of IL-5, and/or increases inhibition of mast cell activation. In some embodiments, “agonize” refers to an increase in a Siglec-8 activity compared to the Siglec-8 activity in the absence of the modulator.

As used herein, the term “epitope” refers to a site on a target molecule (for example, an antigen, such as a protein, nucleic acid, carbohydrate or lipid) to which an antigen-binding molecule (for example, a sdAb or VHH-containing polypeptide) binds. Epitopes often include a chemically active surface grouping of molecules such as amino acids, polypeptides or sugar side chains and have specific three-dimensional structural characteristics as well as specific charge characteristics. Epitopes can be formed both from contiguous and/or juxtaposed noncontiguous residues (for example, amino acids, nucleotides, sugars, lipid moiety) of the target molecule. Epitopes formed from contiguous residues (for example, amino acids, nucleotides, sugars, lipid moiety) typically are retained on exposure to denaturing solvents whereas epitopes formed by tertiary folding typically are lost on treatment with denaturing solvents. An epitope may include but is not limited to at least 3, at least 5 or 8-10 residues (for example, amino acids or nucleotides). In some embodiments, an epitope is less than 20 residues (for example, amino acids or nucleotides) in length, less than 15 residues or less than 12 residues. Two antibodies may bind the same epitope within an antigen if they exhibit competitive binding for the antigen. In some embodiments, an epitope can be identified by a certain minimal distance to a CDR residue on the antigen-binding molecule. In some embodiments, an epitope can be identified by the above distance, and further limited to those residues involved in a bond (for example, a hydrogen bond) between a residue of the antigen-binding molecule and an antigen residue. An epitope can be identified by various scans as well, for example an alanine or arginine scan can indicate one or more residues that the antigen-binding molecule can interact with. Unless explicitly denoted, a set of residues as an epitope does not exclude other residues from being part of the epitope for a particular antigen-binding molecule. Rather, the presence of such a set designates a minimal series (or set of species) of epitopes. Thus, in some embodiments, a set of residues identified as an epitope designates a minimal epitope of relevance for the antigen, rather than an exclusive list of residues for an epitope on an antigen.

A “nonlinear epitope” or “conformational epitope” comprises noncontiguous polypeptides, amino acids and/or sugars within the antigenic protein to which an antigen-binding molecule specific to the epitope binds. In some embodiments, at least one of the residues will be noncontiguous with the other noted residues of the epitope; however, one or more of the residues can also be contiguous with the other residues.

A “linear epitope” comprises contiguous polypeptides, amino acids and/or sugars within the antigenic protein to which an antigen-binding molecule specific to the epitope binds. It is noted that, in some embodiments, not every one of the residues within the linear epitope need be directly bound (or involved in a bond) by the antigen-binding molecule. In some embodiments, linear epitopes can be from immunizations with a peptide that effectively consisted of the sequence of the linear epitope, or from structural sections of a protein that are relatively isolated from the remainder of the protein (such that the antigen-binding molecule can interact, at least primarily), just with that sequence section.

The term “antibody” is used in the broadest sense and encompass various polypeptides that comprise antibody-like antigen-binding domains, including but not limited to conventional antibodies (typically comprising at least one heavy chain and at least one light chain, more typically comprising two heavy chains and two light chains that form two antigen-binding domains), single-domain antibodies (sdAbs, comprising, e.g., at least one VHH domain and an Fc region), VHH-containing polypeptides (polypeptides comprising at least one VHH domain), and fragments of any of the foregoing so long as they exhibit the desired antigen-binding activity. In some embodiments, an antibody comprises a dimerization domain. Such dimerization domains include, but are not limited to, heavy chain constant domains (comprising CH1, hinge, CH2, and CH3, where CH1 typically pairs with a light chain constant domain, CL, to mediate dimerization) and Fc regions (comprising hinge, CH2, and CH3, where the hinge and CH3 mediate dimerization). The term antibody also includes, but is not limited to, chimeric antibodies, humanized antibodies, and antibodies of various species such as camelid (including llama), shark, mouse, human, cynomolgus monkey, etc.

The term “antigen-binding domain” as used herein refers to a portion of an antibody sufficient to bind antigen. In some embodiments, an antigen binding domain of a conventional antibody comprises three heavy chain CDRs and three light chain CDRs. Thus, in some embodiments, an antigen binding domain comprises a heavy chain variable region comprising CDR1-FR2-CDR2-FR3-CDR3, and any portions of FR1 and/or FR4 required to maintain binding to antigen, and a light chain variable region comprising CDR1-FR2-CDR2-FR3-CDR3, and any portions of FR1 and/or FR4 required to maintain binding to antigen. In some embodiments, an antigen-binding domain comprises a heavy chain variable region and a light chain variable region. Nonlimiting such antigen-binding domains include Fabs and scFvs. In some embodiments, an antigen-binding domain comprises a VHH domain. In some embodiments, an antigen-binding domain of an sdAb or VHH-containing polypeptide comprises three CDRs of a VHH domain. Thus, in some embodiments, an antigen binding domain of an sdAb or VHH-containing polypeptide comprises a VHH domain comprising CDR1-FR2-CDR2-FR3-CDR3, and any portions of FR1 and/or FR4 required to maintain binding to antigen.

The term “VHH” or “VHH domain” as used herein refers to the antigen-binding portion of a single-domain antibody, such as a camelid antibody. In some embodiments, a VHH comprises three CDRs and four framework regions, designated FR1, CDR1, FR2, CDR2, FR3, CDR3, and FR4. In some embodiments, a VHH may be truncated at the N-terminus or C-terminus such that it comprises only a partial FR1 and/or FR4, or lacks one or both of those framework regions, so long as the VHH substantially maintains antigen binding and specificity.

The terms “single domain antibody” and “sdAb” are used interchangeably herein to refer to an antibody comprising at least one monomeric domain, such as a VHH domain, without a light chain, and an Fc region. In some embodiments, an sdAb is a dimer of two polypeptides wherein each polypeptide comprises at least one VHH domain and an Fc region. As used herein, the terms “single domain antibody” and “sdAb” encompass polypeptides that comprise multiple VHH domains, such as a polypeptide having the structure VHH₁-VHH₂-Fc or VHH₁-VHH₂-VHH₃-Fc, wherein VHH₁, VHH₂, and VHH₃ may be the same or different.

The term “VHH-containing polypeptide” refers to a polypeptide that comprises at least one VHH domain. In some embodiments, a VHH polypeptide comprises two, three, or four or more VHH domains, wherein each VHH domain may be the same or different. In some embodiments, a VHH-containing polypeptide comprises an Fc region. In some such embodiments, the VHH-containing polypeptide may be referred to as an sdAb. Further, in some such embodiments, the VHH polypeptide may form a dimer. Nonlimiting structures of VHH-containing polypeptides, which are also sdAbs, include VHH₁-Fc, VHH₁-VHH₂-Fc, and VHH₁-VHH₂-VHH₃-Fc, wherein VHH₁, VHH₂, and VHH₃ may be the same or different. In some embodiments of such structures, one VHH may be connected to another VHH by a linker, or one VHH may be connected to the Fc region by a linker. In some such embodiments, the linker comprises 1-20 amino acids, preferably 1-20 amino acids predominantly composed of glycine and, optionally, serine. Non-limiting linkers are presented in SEQ ID NOs: 69 and 70. In some embodiments, when a VHH-containing polypeptide comprises an Fc, it forms a dimer. Thus, the structure VHH₁-VHH₂-Fc, if it forms a dimer, is considered to be tetravalent (i.e., the dimer has four VHH domains). Similarly, the structure VHH₁-VHH₂-VHH₃-Fc, if it forms a dimer, is considered to be hexavalent (i.e., the dimer has six VHH domains). In some embodiments a VHH-containing polypeptide comprising an Fc region forms a homodimer, e.g., through association of the hinge of the Fc regions.

The term “monoclonal antibody” refers to an antibody (including an sdAb or VHH-containing polypeptide) of a substantially homogeneous population of antibodies, that is, the individual antibodies comprising the population are identical except for possible naturally-occurring mutations that may be present in minor amounts. Monoclonal antibodies are highly specific, being directed against a single antigenic site. Furthermore, in contrast to polyclonal antibody preparations, which typically include different antibodies directed against different determinants (epitopes), each monoclonal antibody is directed against a single determinant on the antigen. Thus, a sample of monoclonal antibodies can bind to the same epitope on the antigen. The modifier “monoclonal” indicates the character of the antibody as being obtained from a substantially homogeneous population of antibodies, and is not to be construed as requiring production of the antibody by any particular method. For example, the monoclonal antibodies may be made by the hybridoma method first described by Kohler and Milstein, 1975, Nature 256:495, or may be made by recombinant DNA methods such as described in U.S. Pat. No. 4,816,567. The monoclonal antibodies may also be isolated from phage libraries generated using the techniques described in McCafferty et al., 1990, Nature 348:552-554, for example.

The term “CDR” denotes a complementarity determining region as defined by at least one manner of identification to one of skill in the art. In some embodiments, CDRs can be defined in accordance with any of the Chothia numbering schemes (see Chothia and Lesk, J Mol Biol, 1987, 196:901-917), the Kabat numbering scheme (see Kabat et al., 1992, Sequences of Proteins of Immunological Interest, DIANE Publishing: 2719), a combination of Kabat and Chothia, the AbM definition (see Whitelegg & Rees, Protein Eng. 2000, 13:819-824; Whitelegg & Rees, Methods Mol Biol. 2004, 248:51-91), the IMGT definition (see Lefranc, (1999) The Immunologist, 7, 132-136), and/or the Contact definition (see MacCallum et al., J. Mol. Biol. 1996, 262:732-745). A VHH comprises three CDRs, designated CDR1, CDR2, and CDR3.

The term “heavy chain constant region” as used herein refers to a region comprising at least three heavy chain constant domains, CH1, hinge, CH2, and CH3. Of course, non-function-altering deletions and alterations within the domains are encompassed within the scope of the term “heavy chain constant region,” unless designated otherwise. Nonlimiting exemplary heavy chain constant regions include γ, δ, and α. Nonlimiting exemplary heavy chain constant regions also include ε and μ. Each heavy constant region corresponds to an antibody isotype. For example, an antibody comprising a γ constant region is an IgG antibody, an antibody comprising a δ constant region is an IgD antibody, and an antibody comprising an α constant region is an IgA antibody. Further, an antibody comprising a μ constant region is an IgM antibody, and an antibody comprising an ε constant region is an IgE antibody. Certain isotypes can be further subdivided into subclasses. For example, IgG antibodies include, but are not limited to, IgG1 (comprising a γ₁ constant region), IgG2 (comprising a γ₂ constant region), IgG3 (comprising a γ₃ constant region), and IgG4 (comprising a γ₄ constant region) antibodies; IgA antibodies include, but are not limited to, IgA1 (comprising an α₁ constant region) and IgA2 (comprising an α₂ constant region) antibodies; and IgM antibodies include, but are not limited to, IgM1 and IgM2. For all heavy chain constant region amino acid positions discussed in the present invention, numbering is according to the EU index first described in Edelman et al., 1969, Proc. Natl. Acad. Sci. USA 63(1):78-85, describing the amino acid sequence of myeloma protein Eu, which is the first human IgG1 sequenced. The Eu index of Edelman et al. is also set forth in Kabat et al., 1991, Sequences of Proteins of Immunological Interest, 5th Ed., United States Public Health Service, National Institutes of Health, Bethesda. Thus, the phrases “EU index as set forth in Kabat” or “EU index of Kabat” and “position . . . according to the EU index as set forth in Kabat,” and grammatical variants thereof refer to the residue numbering system based on the human lgG1 Eu antibody of Edelman et al. as set forth in Kabat 1991.

An “Fc region” as used herein refers to a portion of a heavy chain constant region comprising CH2 and CH3. In some embodiments, an Fc region comprises a hinge, CH2, and CH3. In various embodiments, when an Fc region comprises a hinge, the hinge and CH3 mediate dimerization between two Fc-containing polypeptides. An Fc region may be of any antibody heavy chain constant region isotype discussed herein. In some embodiments, an Fc region is from an IgG1, IgG2, IgG3, or IgG4.

An “acceptor human framework” as used herein is a framework comprising the amino acid sequence of a heavy chain variable domain (V_{H}) framework derived from a human immunoglobulin framework or a human consensus framework, as discussed herein. An acceptor human framework derived from a human immunoglobulin framework or a human consensus framework can comprise the same amino acid sequence thereof, or it can contain amino acid sequence changes. In some embodiments, the number of amino acid changes are fewer than 10, or fewer than 9, or fewer than 8, or fewer than 7, or fewer than 6, or fewer than 5, or fewer than 4, or fewer than 3, across all of the human frameworks in a single antigen binding domain, such as a VHH.

“Affinity” refers to the strength of the sum total of noncovalent interactions between a single binding site of a molecule (for example, an antibody, such as an sdAb, or VHH-containing polypeptide) and its binding partner (for example, an antigen). The affinity or the apparent affinity of a molecule X for its partner Y can generally be represented by the dissociation constant (K_{d}) or the K_{d-apparent}, respectively. Affinity can be measured by common methods known in the art (such as, for example, ELISA K_{d}, KinExA, flow cytometry, and/or surface plasmon resonance devices), including those described herein. Such methods include, but are not limited to, methods involving BIAcore®, Octet®, or flow cytometry. In some embodiments, an exemplary polypeptide binds to Siglec-8 with a K_{D} of less than or equal to about 90, 85, 80, 75, 70, 68, 65, 60, 55, 56, or 50×10⁻¹² M, as measured by surface plasmon resonance (SPR) assay. In some embodiments, an exemplary polypeptide binds to Siglec-8 with a K_{D} of less than or equal to about 500, 400, 300, 200, 100, 95, 90, 85, 80, 75, 70, 68, 65, 60, 55, 45, 40, 35, 30, 25, 20, 15, or 10×10⁻¹² M, as measured by surface plasmon resonance (SPR) assay.

The term “K_{d}”, as used herein, refers to the equilibrium dissociation constant of an antigen-binding molecule/antigen interaction. When the term “K_{d}” is used herein, it includes K_{d} and K_{d-apparent}.

In some embodiments, the K_{d} of the antigen-binding molecule is measured by flow cytometry using an antigen-expressing cell line and fitting the mean fluorescence measured at each antibody concentration to a non-linear one-site binding equation (Prism Software graphpad). In some such embodiments, the K_{d} is K_{d-apparent}.

The term “biological activity” refers to any one or more biological properties of a molecule (whether present naturally as found in vivo, or provided or enabled by recombinant means).

An “agonist” or “activating” antibody is one that increases and/or activates a biological activity of the target antigen. In some embodiments, the agonist antibody binds to an antigen and increases its biologically activity by at least about 20%, 40%, 60%, 80%, 85% or more.

An “antagonist”, a “blocking” or “neutralizing” antibody is one that inhibits a biological activity of the target antigen. In some embodiments, a neutralizing antibody binds to an antigen and reduces its biologically activity by at least about 20%, 40%, 60%, 80%, 85% 90%, 95%, 99% or more.

An “affinity matured” sdAb or VHH-containing polypeptide refers to a sdAb or VHH-containing polypeptide with one or more alterations in one or more CDRs compared to a parent sdAb or VHH-containing polypeptide that does not possess such alterations, such alterations resulting in an improvement in the affinity of the sdAb or VHH-containing polypeptide for antigen.

A “humanized VHH” as used herein refers to a VHH in which one or more framework regions have been substantially replaced with human framework regions. In some instances, certain framework region (FR) residues of the human immunoglobulin are replaced by corresponding non-human residues. Furthermore, the humanized VHH can comprise residues that are found neither in the original VHH nor in the human framework sequences, but are included to further refine and optimize sdAb VHH-containing polypeptide performance. In some embodiments, a humanized sdAb or VHH-containing polypeptide comprises a human Fc region. As will be appreciated, a humanized sequence can be identified by its primary sequence and does not necessarily denote the process by which the antibody was created.

An “effector-positive Fc region” possesses an “effector function” of a native sequence Fc region. Exemplary “effector functions” include Fc receptor binding; Clq binding and complement dependent cytotoxicity (CDC); Fc receptor binding; antibody-dependent cell-mediated cytotoxicity (ADCC); phagocytosis; down regulation of cell surface receptors (for example B-cell receptor); and B-cell activation, etc. Such effector functions generally require the Fc region to be combined with a binding domain (for example, an antibody variable domain) and can be assessed using various assays.

A “native sequence Fc region” comprises an amino acid sequence identical to the amino acid sequence of an Fc region found in nature. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof.

A “variant Fc region” comprises an amino acid sequence which differs from that of a native sequence Fc region by virtue of at least one amino acid modification. In some embodiments, a “variant Fc region” comprises an amino acid sequence which differs from that of a native sequence Fc region by virtue of at least one amino acid modification, yet retains at least one effector function of the native sequence Fc region. In some embodiments, the variant Fc region has at least one amino acid substitution compared to a native sequence Fc region or to the Fc region of a parent polypeptide, for example, from about one to about ten amino acid substitutions, and preferably, from about one to about five amino acid substitutions in a native sequence Fc region or in the Fc region of the parent polypeptide. In some embodiments, the variant Fc region herein will possess at least about 80% sequence identity with a native sequence Fc region and/or with an Fc region of a parent polypeptide, at least about 90% sequence identity therewith, at least about 95%, at least about 96%, at least about 97%, at least about 98%, or at least about 99% sequence identity therewith.

“Fc receptor” or “FcR” describes a receptor that binds to the Fc region of an antibody. In some embodiments, an FcγR is a native human FcR. In some embodiments, an FcR is one which binds an IgG antibody (a gamma receptor) and includes receptors of the FcγRI, FcγRII, and FcγRIII subclasses, including allelic variants and alternatively spliced forms of those receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain Inhibiting receptor FcγRIIB contains an immunoreceptor tyrosine-based inhibition motif (ITIM) in its cytoplasmic domain. (See, for example, Daeron, Annu. Rev. Immunol. 15:203-234 (1997)). FcRs are reviewed, for example, in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. For example, the term “Fc receptor” or “FcR” also includes the neonatal receptor, FcRn, which is responsible for the transfer of maternal IgGs to the fetus (Guyer et al., J. Immunol. 117:587 (1976) and Kim et al., J. Immunol. 24:249 (1994)) and regulation of homeostasis of immunoglobulins. Methods of measuring binding to FcRn are known (see, for example, Ghetie and Ward, Immunol. Today 18(12):592-598 (1997); Ghetie et al., Nature Biotechnology, (1997); Hinton et al., J. Biol. Chem. 279(8):6213-6216 (2004); WO 2004/92219 (Hinton et al.).

The term “substantially similar” or “substantially the same,” as used herein, denotes a sufficiently high degree of similarity between two or more numeric values such that one of skill in the art would consider the difference between the two or more values to be of little or no biological and/or statistical significance within the context of the biological characteristic measured by said value. In some embodiments the two or more substantially similar values differ by no more than about any one of 5%, 10%, 15%, 20%, 25%, or 50%.

A polypeptide “variant” means a biologically active polypeptide having at least about 80% amino acid sequence identity with the native sequence polypeptide after aligning the sequences and introducing gaps, if necessary, to achieve the maximum percent sequence identity, and not considering any conservative substitutions as part of the sequence identity. Such variants include, for instance, polypeptides wherein one or more amino acid residues are added, or deleted, at the N- or C-terminus of the polypeptide. In some embodiments, a variant will have at least about 80% amino acid sequence identity. In some embodiments, a variant will have at least about 90% amino acid sequence identity. In some embodiments, a variant will have at least about 95% amino acid sequence identity with the native sequence polypeptide.

As used herein, “percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared.

An amino acid substitution may include but are not limited to the replacement of one amino acid in a polypeptide with another amino acid. Exemplary substitutions are shown in Table 1. Amino acid substitutions may be introduced into an antibody of interest and the products screened for a desired activity, for example, retained/improved antigen binding, decreased immunogenicity, or improved ADCC or CDC.

TABLE 1

| Original Residue | Exemplary Substitutions |
|---|---|
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe; Norleucine |
| Leu (L) | Norleucine; Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala; Norleucine |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

The term “vector” is used to describe a polynucleotide that can be engineered to contain a cloned polynucleotide or polynucleotides that can be propagated in a host cell. A vector can include one or more of the following elements: an origin of replication, one or more regulatory sequences (such as, for example, promoters and/or enhancers) that regulate the expression of the polypeptide of interest, and/or one or more selectable marker genes (such as, for example, antibiotic resistance genes and genes that can be used in colorimetric assays, for example, β-galactosidase). The term “expression vector” refers to a vector that is used to express a polypeptide of interest in a host cell.

A “host cell” refers to a cell that may be or has been a recipient of a vector or isolated polynucleotide. Host cells may be prokaryotic cells or eukaryotic cells. Exemplary eukaryotic cells include mammalian cells, such as primate or non-primate animal cells; fungal cells, such as yeast; plant cells; and insect cells. Nonlimiting exemplary mammalian cells include, but are not limited to, NSO cells, PER.C6® cells (Crucell), and 293 and CHO cells, and their derivatives, such as 293-6E, CHO-DG44, CHO-K1, CHO-S, and CHO-DS cells. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) a provided herein.

The term “isolated” as used herein refers to a molecule that has been separated from at least some of the components with which it is typically found in nature or produced. For example, a polypeptide is referred to as “isolated” when it is separated from at least some of the components of the cell in which it was produced. Where a polypeptide is secreted by a cell after expression, physically separating the supernatant containing the polypeptide from the cell that produced it is considered to be “isolating” the polypeptide. Similarly, a polynucleotide is referred to as “isolated” when it is not part of the larger polynucleotide (such as, for example, genomic DNA or mitochondrial DNA, in the case of a DNA polynucleotide) in which it is typically found in nature, or is separated from at least some of the components of the cell in which it was produced, for example, in the case of an RNA polynucleotide. Thus, a DNA polynucleotide that is contained in a vector inside a host cell may be referred to as “isolated”.

The terms “individual” and “subject” are used interchangeably herein to refer to an animal; for example, a mammal. In some embodiments, methods of treating mammals, including, but not limited to, humans, rodents, simians, felines, canines, equines, bovines, porcines, ovines, caprines, mammalian laboratory animals, mammalian farm animals, mammalian sport animals, and mammalian pets, are provided. In some examples, an “individual” or “subject” refers to an individual or subject in need of treatment for a disease or disorder. In some embodiments, the subject to receive the treatment can be a patient, designating the fact that the subject has been identified as having a disorder of relevance to the treatment, or being at adequate risk of contracting the disorder.

A “disease” or “disorder” as used herein refers to a condition where treatment is needed and/or desired.

In some embodiments, an “increase” or “decrease” refers to a statistically significant increase or decrease, respectively. As will be clear to the skilled person, “modulating” can also involve effecting a change (which can either be an increase or a decrease) in affinity, avidity, specificity and/or selectivity of a target or antigen, for one or more of its ligands, binding partners, partners for association into a homomultimeric or heteromultimeric form, or substrates; effecting a change (which can either be an increase or a decrease) in the sensitivity of the target or antigen for one or more conditions in the medium or surroundings in which the target or antigen is present (such as pH, ion strength, the presence of co-factors, etc.); and/or cellular proliferation or cytokine production, compared to the same conditions but without the presence of a test agent. This can be determined in any suitable manner and/or using any suitable assay known per se or described herein, depending on the target involved.

As used herein, “an immune response” is meant to encompass cellular and/or humoral immune responses that are sufficient to inhibit or prevent onset or ameliorate the symptoms of disease (for example, cancer or cancer metastasis). “An immune response” can encompass aspects of both the innate and adaptive immune systems.

As used herein, “treatment” is an approach for obtaining beneficial or desired clinical results. “Treatment” as used herein, covers any administration or application of a therapeutic for disease in a mammal, including a human. For purposes of this disclosure, beneficial or desired clinical results include, but are not limited to, any one or more of: alleviation of one or more symptoms, diminishment of extent of disease, preventing exacerbation of disease, preventing or delaying recurrence of disease, delay or slowing of disease progression, amelioration of the disease state, inhibiting the disease or progression of the disease, and inhibiting or slowing the disease or its progression. Also encompassed by “treatment” is a reduction of pathological consequence of a disease. The methods provided herein contemplate any one or more of these aspects of treatment. In-line with the above, the term treatment does not require one-hundred percent removal of all aspects of the disorder.

“Ameliorating” means a lessening or improvement of one or more symptoms as compared to not administering a therapeutic agent. “Ameliorating” also includes shortening or reduction in duration of a symptom.

The term “biological sample” means a quantity of a substance from a living thing or formerly living thing. Such substances include, but are not limited to, blood, (for example, whole blood), plasma, serum, bronchoalveolar lavage fluid, sputum, nasal lavage fluid, urine, amniotic fluid, synovial fluid, endothelial cells, leukocytes, monocytes, other cells, organs, tissues, bone marrow, lymph nodes and spleen.

The term “control” or “reference” in the context of an experiment or comparison, refers to a composition known to not contain an analyte (“negative control”) or to contain an analyte (“positive control”). A positive control can comprise a known concentration of analyte. A control or reference may also refer to a control agent known to lack the activity of an agent being tested, such as an antibody.

“Preventing,” as used herein, includes providing prophylaxis with respect to the occurrence or recurrence of a disease in a subject that may be predisposed to the disease but has not yet been diagnosed with the disease. Unless otherwise specified, the terms “reduce”, “inhibit”, or “prevent” do not denote or require complete prevention over all time, but just over the time period being measured.

A “therapeutically effective amount” of a substance/molecule, agonist or antagonist may vary according to factors such as the disease state, age, sex, and weight of the individual, and the ability of the substance/molecule, agonist or antagonist to elicit a desired response in the individual. A therapeutically effective amount is also one in which any toxic or detrimental effects of the substance/molecule, agonist or antagonist are outweighed by the therapeutically beneficial effects. A therapeutically effective amount may be delivered in one or more administrations. A therapeutically effective amount refers to an amount effective, at dosages and for periods of time necessary, to achieve the desired therapeutic and/or prophylactic result.

The terms “pharmaceutical formulation” and “pharmaceutical composition” are used interchangeably and refer to a preparation which is in such form as to permit the biological activity of the active ingredient(s) to be effective, and which contains no additional components which are unacceptably toxic to a subject to which the formulation would be administered. Such formulations may be sterile.

A “pharmaceutically acceptable carrier” refers to a non-toxic solid, semisolid, or liquid filler, diluent, encapsulating material, formulation auxiliary, or carrier conventional in the art for use with a therapeutic agent that together comprise a “pharmaceutical composition” for administration to a subject. A pharmaceutically acceptable carrier is non-toxic to recipients at the dosages and concentrations employed and are compatible with other ingredients of the formulation. The pharmaceutically acceptable carrier is appropriate for the formulation employed.

Administration “in combination with” one or more further therapeutic agents includes simultaneous (concurrent) and sequential administration in any order.

The term “concurrently” is used herein to refer to administration of two or more therapeutic agents, where at least part of the administration overlaps in time, or where the administration of one therapeutic agent falls within a short period of time relative to administration of the other therapeutic agent, or wherein the therapeutic effects of both agents overlap for at least a period of time.

The term “sequentially” is used herein to refer to administration of two or more therapeutic agents that does not overlap in time, or wherein the therapeutic effects of the agents do not overlap.

As used herein, “in conjunction with” refers to administration of one treatment modality in addition to another treatment modality. As such, “in conjunction with” refers to administration of one treatment modality before, during, or after administration of the other treatment modality to the individual.

The term “package insert” is used to refer to instructions customarily included in commercial packages of therapeutic products, that contain information about the indications, usage, dosage, administration, combination therapy, contraindications and/or warnings concerning the use of such therapeutic products.

An “article of manufacture” is any manufacture (for example, a package or container) or kit comprising at least one reagent, for example, a medicament for treatment of a disease or disorder (for example, cancer), or a probe for specifically detecting a biomarker described herein. In some embodiments, the manufacture or kit is promoted, distributed, or sold as a unit for performing the methods described herein.

The terms “label” and “detectable label” mean a moiety attached, for example, to an antibody or antigen to render a reaction (for example, binding) between the members of the specific binding pair, detectable. The labeled member of the specific binding pair is referred to as “detectably labeled.” Thus, the term “labeled binding protein” refers to a protein with a label incorporated that provides for the identification of the binding protein. In some embodiments, the label is a detectable marker that can produce a signal that is detectable by visual or instrumental means, for example, incorporation of a radiolabeled amino acid or attachment to a polypeptide of biotinyl moieties that can be detected by marked avidin (for example, streptavidin containing a fluorescent marker or enzymatic activity that can be detected by optical or colorimetric methods). Examples of labels for polypeptides include, but are not limited to, the following: radioisotopes or radionuclides (for example, ³H, ¹⁴C, ³⁵S, ⁹⁰Y, ⁹⁹Tc, ¹¹¹In, ¹²⁵I, ¹³¹I, ¹⁷⁷Lu, ¹⁶⁶Ho, or ¹⁵³Sm); chromogens, fluorescent labels (for example, FITC, rhodamine, lanthanide phosphors), enzymatic labels (for example, horseradish peroxidase, luciferase, alkaline phosphatase); chemiluminescent markers; biotinyl groups; predetermined polypeptide epitopes recognized by a secondary reporter (for example, leucine zipper pair sequences, binding sites for secondary antibodies, metal binding domains, epitope tags); and magnetic agents, such as gadolinium chelates. Representative examples of labels commonly employed for immunoassays include moieties that produce light, for example, acridinium compounds, and moieties that produce fluorescence, for example, fluorescein. In this regard, the moiety itself may not be detectably labeled but may become detectable upon reaction with yet another moiety.

### Exemplary Siglec-8 Binding Polypeptides and Proteins

Siglec-8 binding polypeptides and proteins are provided herein. In various embodiments, the Siglec-8 binding polypeptide or protein is a Siglec-8 agonist. In some embodiments, a Siglec-8 binding polypeptide is provided that comprises at least two antigen-binding domains that bind Siglec-8. In some embodiments, a Siglec-8 binding polypeptide or protein is provided that comprises at least three antigen-binding domains that bind Siglec-8. In some embodiments, a Siglec-8 binding polypeptide is provided that comprises at least two antigen-binding domains that bind Siglec-8 and a dimerization domain. In some embodiments, a Siglec-8 binding polypeptide is provided that comprises more than three antigen-binding domains that bind Siglec-8 and a dimerization domain. In some embodiments, the dimerization domain is a Fc region. In some embodiments, the polypeptide mediates eosinophil killing in the presence of IL-5 with a lower EC50 than a polypeptide that comprises two antigen-binding domains that bind Siglec-8 under physiological conditions, such as a conventional antibody. In some embodiments, two or more antigen-binding domains are the same. In some embodiments, two or more antigen-binding domains are different. In some embodiments, all the antigen-binding domains are the same. In some embodiments, each antigen-binding domain comprises a heavy chain variable region and a light chain variable region. In some embodiments, each antigen-binding domain is a Fab or scFv.

In various embodiments, the Siglec-8 binding polypeptides or proteins comprise at least one VHH domain that binds Siglec-8. In some embodiments, the Siglec-8-binding polypeptides or proteins bind human Siglec-8. In some embodiments, a Siglec-8 binding polypeptide or protein binds Siglec-8 and agonizes (i.e., increases the activity of) Siglec-8. In some embodiments, a Siglec-8 binding polypeptide or protein agonizes Siglec-8, resulting in killing of eosinophils in the presence of IL-5. In some embodiments, a Siglec-8 binding polypeptide or protein depletes eosinophils in vitro or in vivo in the presence of IL-5.

In some embodiments, a Siglec-8 binding polypeptide provided herein comprises one, two, three, four, five, six, seven, or eight VHH domains that bind Siglec-8. In some embodiments, a Siglec-8 binding polypeptide provided herein comprises one, two, three, or four VHH domains that bind Siglec-8. In some embodiments, a Siglec-8 binding polypeptide provided herein comprises two or three VHH domains that bind Siglec-8. Siglec-8 binding polypeptides may comprise one or more VHH domains that bind one or more target proteins other than Siglec-8. Such polypeptides may be referred to as “multispecific” polypeptides.

In various embodiments, a Siglec-8 binding polypeptide comprises one, two, three, or four VHH domains that bind Siglec-8. In various embodiments, a Siglec-8 binding polypeptide comprises two or three VHH domains that bind Siglec-8. In some embodiment, a Siglec-8 binding polypeptide comprises two, three, or four VHH domains that bind Siglec-8 operably linked to each other via a peptide linker. A non-limiting example of a linker which may be used to link VHH domains is presented in SEQ ID NO: 69.

In some embodiments, a Siglec-8 binding polypeptide comprises at least one VHH domain that binds Siglec-8 and an Fc region. In some embodiments, a Siglec-8 binding polypeptide provided herein comprises one, two, three, or four VHH domains that bind Siglec-8 and an Fc region. In some embodiments, a Siglec-8 binding polypeptide provided herein comprises two or three VHH domains that bind Siglec-8 and an Fc region. In some embodiments, an Fc region mediates dimerization of the Siglec-8 binding polypeptide at physiological conditions such that a dimer is formed that doubles the number of Siglec-8 binding sites in the protein. For example, a Siglec-8 binding polypeptide comprising three VHH domains that bind Siglec-8 and an Fc region is trivalent as a monomer, but at physiological conditions, the Fc region may mediate dimerization, to form a Siglec-8 binding protein as a hexavalent dimer under such conditions. In some embodiments, the Fc region is a human IgG1 Fc region variant, such as any human IgG1 Fc region variant described herein.

In some embodiments, a Siglec-8 binding polypeptide comprises at least one VHH domain described herein fused to an Fc region. In some embodiments, the Fc region comprises the sequence of SEQ ID NO: 44, 45, 65, 66, 67, or 68. In some embodiment, a Siglec-8 binding polypeptide comprises at least one VHH domain described herein operably linked to an Fc region via a peptide linker. A non-limiting example of a linker which may be used to link a VHH domain and an Fc region is presented in SEQ ID NO: 70.

In various embodiments, an Fc region included in a Siglec-8 binding polypeptide is a human Fc region, or is derived from a human Fc region. In some embodiments, the Fc region is an IgG1 isotype, IgG2 isotype, IgG3 isotype, or IgG4 isotype. In some embodiments, the Fc region is a wild type IgG1 isotype (see, e.g., SEQ ID NOs: 45 and 66)

In some embodiments, the Fc region included in a Siglec-8 binding polypeptide is derived from a human Fc region and comprises mutations designed for heterodimerization.

In some embodiments, the Fc region included in a Siglec-8 binding polypeptide is derived from a human Fc region and comprises mutations designed to alter antibody-dependent cellular cytotoxicity (ADCC) and/or complement-dependent cytotoxicity (CDC), e.g., the amino acid modifications described in WO 00/42072, WO 04/029207, WO 04/099249, and WO 04/063351. In some embodiments, the Fc region included in a Siglec-8 binding polypeptide include a deletion of Glu233, Leu234, Leu235, or any combination thereof, according to the EU index as set forth in Kabat. In some embodiments, the Fc region included in a Siglec-8 binding polypeptide include a deletion of Glu233, Leu234, and Leu235 (xELL) according to the EU index as set forth in Kabat (see, e.g., SEQ ID NOs: 45 and 67), to reduce or eliminate ADCC. In some embodiments, the Fc region included in a Siglec-8 binding polypeptide include a modification at Ser239 and/or Ile332, according to the EU index as set forth in Kabat. In one embodiment the Fc region included in a Siglec-8 binding polypeptide comprises the following modifications Ser239Asp and Ile332Glu (S239D, 1332E) according to the EU index as set forth in Kabat to enhance ADCC (see, e.g., SEQ ID NOs:65 and 68). In some embodiments, the Fc region lacks Lys447 according to the EU index as set forth in Kabat (see, e.g., SEQ ID NOs: 66, 67, and 68).

Fc regions that can be used in a Siglec-8 binding polypeptide include Fc regions comprising the amino acid sequence of SEQ ID NOs: 44, 45, 65, 66, 67, or 68.

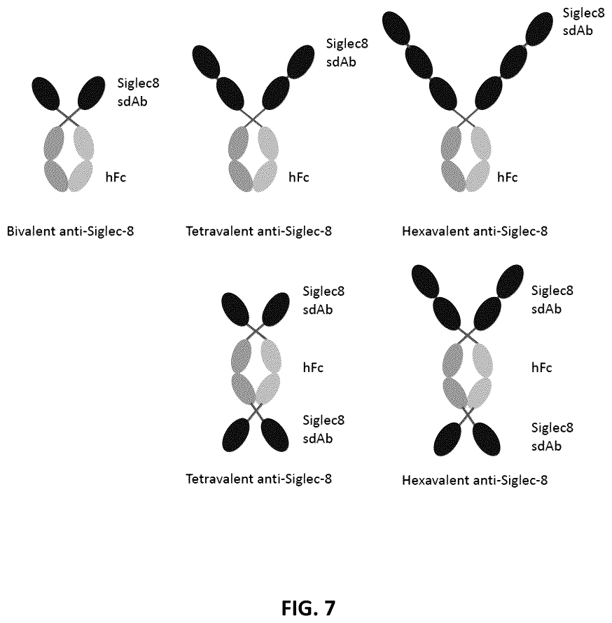

The Siglec-8 binding proteins disclosed herein can take various formats, for example, the formats depicted in FIG. 7. In some embodiments, the protein is a homodimer comprising two identical polypeptide chains, which comprises one or more, two or more, three or more, two, three, or two or three VHH domains that bind Siglec-8 and an Fc region. In some embodiments, the polypeptide chain comprises, from the N-terminus to the C-terminus, a VHH domain that binds Siglec-8 and an Fc region. In some embodiments, the polypeptide chain comprises, from the N-terminus to the C-terminus, a first VHH domain that binds Siglec-8, a second VHH domain that binds Siglec-8, and an Fc region, wherein the first and second VHH domains can be identical or different. In some embodiments, the polypeptide chain comprises, from the N-terminus to the C-terminus, a first VHH domain that binds Siglec-8, an Fc region, and a second VHH domain that binds Siglec-8, wherein the first and second VHH domains can be identical or different. In some embodiments, the polypeptide chain comprises, from the N-terminus to the C-terminus, a first VHH domain that binds Siglec-8, a second VHH domain that binds Siglec-8, a third VHH domain that binds Siglec-8, and an Fc region, wherein the first, second, and third VHH domains can be identical or different. In some embodiments, the polypeptide chain comprises, from the N-terminus to the C-terminus, a first VHH domain that binds Siglec-8, a second VHH domain that binds Siglec-8, an Fc region, and a third VHH domain that binds Siglec-8, wherein the first, second, and third VHH domains can be identical or different.

In some embodiments, where a Siglec-8 binding polypeptide or protein containing an Fc domain that has ADCC effector function, the Siglec-8 binding polypeptide or protein increases killing of eosinophils by NK cells in the absence of IL-5.

In some embodiments, a Siglec-8 binding polypeptide comprises at least two VHH domains, wherein a first VHH domain binds Siglec-8 and a second VHH domain binds an antigen other than Siglec-8. Such polypeptides may be referred to as “bispecific” or “multispecific.”

Nonlimiting exemplary Siglec-8 binding VHH domains are described in the “Siglec-8 binding VHH domains” subsection below.

### Siglec-8 Binding VHH Domains

The present disclosure provides VHH domains that bind Siglec-8. Table 2 lists exemplary VHH domains and the CDR sequences in these VHH domains under various CDR definitions. A VHH name that begins with “hz” indicates that it is a humanized version of the corresponding parental polypeptide.

TABLE 2
Polypeptides comprising a VHH that binds Siglec-8

| | SEQ ID NOs | | | | | |
|---|---|---|---|---|---|---|
| | | CDR1, CDR2, CDR3 | | | | |
| Name | VHH | AbM | Chothia | Kabat | Contact | IMGT |
| Siglec8- | 1 | 2, 3, 4 | 74, 80, 4 | 85, 91, 4 | 98, 104, 109 | 114, 120, 125 |
| G2 | | | | | | |
| hzG2v14 | 5 | 6, 7, 8; | 74, 80, 8 | 85, 92, 8 | 98, 104, 110 | 114, 120, 126 |
| | | 2, 3, 8 | | | | |
| hzG2v47 | 11 | 13, 14, 15; | 75, 80, 8 | 86, 92, 8 | 99, 104, 110 | 115, 120, 126 |
| | | 13, 3, 8 | | | | |
| hzG2v51 | 16 | 18, 19, 20; | 76, 80, 8 | 87, 93, 8 | 100, 104, 110 | 116, 120, 126 |
| | | 18, 3, 8 | | | | |
| hzG2v52 | 21 | 23, 24, 25; | 76, 80, 8 | 87, 92, 8 | 100, 104, 110 | 116, 120, 126 |
| | | 18, 3, 8 | | | | |
| hzG2v53 | 26 | 28, 29, 30, | 76, 81, 8 | 87, 94, 8 | 100, 105, 110 | 116, 121, 126 |
| | | 18, 29, 8 | | | | |
| hzG2v54 | 31 | 33, 34, 35 | 76, 80, 8 | 87, 92, 8 | 100, 104, 110 | 116, 120, 126 |
| | | 18, 3, 8 | | | | |
| hzG2v55 | 36 | 38, 39, 40 | 75, 80, 8 | 86, 92, 8 | 99, 104, 110 | 115, 120, 126 |
| | | 13, 3, 8 | | | | |
| Siglec8- | 49 | 52, 53, 54 | 77, 82, 54 | 88, 95, 54 | 101, 106, 111 | 117, 122, 127 |
| B12 | | | | | | |
| Siglec8- | 55 | 58, 59, 60 | 78, 83, 60 | 89, 96, 60 | 102, 107, 112 | 118, 123, 128 |
| D1 | | | | | | |
| Siglec8- | 61 | 63, 64, | 79, 84, | 90, 97, | 103, 108, 113 | 119, 124, 129 |
| E2 | | “GAY” | “GAY” | “GAY” | | |

Where multiple sets of CDR sequence identifiers are provided, e.g., for hzG2v14 under the AbM CDR definition, the multiple sets of CDR sequences refer to identical sequences. In other words, in the hzG2v14 example, SEQ ID NOs: 2 and 6 represent the same sequence, and SEQ ID NOs: 3 and 7 represent the same sequence.

Amino acid sequences of additional exemplary humanized VHH domains are set forth in SEQ ID NOs: 130-138, 140-148, 161, 163, and 164. Although their CDR sequences are not provided in the Sequence Listing, they can be identified by methods known in the art, e.g., using the AbYsis server (www.abysis.org/abysis/). These VHH domains are variants of hzG2v14, hzG2v47, hzG2v51, hzG2v52, hzG2v53, hzG2v54, and hzG2v55 of Table 2.

Therefore, their CDR sequences can also be identified by aligning the VHH amino acid sequences with one or more of those of reference sequences (e.g., hzG2v14) and isolating the regions corresponding to the CDRs of the reference sequences.

In certain embodiments, a Siglec-8 binding VHH domain comprises CDR1, CDR2, and CDR3 sequences of a VHH disclosed herein, e.g., as set forth in a sequence selected from SEQ ID NOs: 26, 5, 11, 16, 21, 31, 36, 130-138, 140-148, 161, 163, and 164. In certain embodiments, the VHH domain comprises CDR1, CDR2, and CDR3 sequences of a VHH disclosed herein under the AbM definition, e.g., a set of CDR sequences disclosed in the “AbM” column of Table 2. In certain embodiments, the VHH domain comprises CDR1, CDR2, and CDR3 sequences of a VHH disclosed herein under the Chothia definition, e.g., a set of CDR sequences disclosed in the “Chothia” column of Table 2. In certain embodiments, the VHH domain comprises CDR1, CDR2, and CDR3 sequences of a VHH disclosed herein under the Kabat definition, e.g., a set of CDR sequences disclosed in the “Kabat” column of Table 2. In certain embodiments, the VHH domain comprises CDR1, CDR2, and CDR3 sequences of a VHH disclosed herein under the Contact definition, e.g., a set of CDR sequences disclosed in the “Contact” column of Table 2. In certain embodiments, the VHH domain comprises CDR1, CDR2, and CDR3 sequences of a VHH disclosed herein under the IMGT definition, e.g., a set of CDR sequences disclosed in the “IMGT” column of Table 2.

In certain embodiments, under the AbM definition, a Siglec-8 binding VHH domain comprises consensus hzG2 CDR1, CDR2, and CDR3 sequences set forth in SEQ ID NOs: 46, 47, and 48, respectively. In certain embodiments, the X₁ in SEQ ID NO: 48 is Q, S, or T. In certain embodiments, the X2 in SEQ ID NO: 48 is T. In various embodiments, the VHH domain comprises the CDR1 sequence of SEQ ID NO: 2, 6, 13, 18, 23, 28, 33, or 38; a CDR2 sequence of SEQ ID NO: 3, 7, 14, 19, 24, 29, 34, or 39; and the CDR3 sequence of SEQ ID NO: 4, 8, 15, 25, 30, 35, or 40. In some embodiments, the VHH domain comprises the CDR1, CDR2, and CDR3 sequences of SEQ ID NOs: 6, 7, and 8. In some embodiments, the VHH domain comprises the CDR1, CDR2, and CDR3 sequences of SEQ ID NOs: 13, 14, and 15. In some embodiments, the VHH domain comprises the CDR1, CDR2, and CDR3 sequences of SEQ ID NOs: 18, 19, and 20. In some embodiments, the VHH domain comprises the CDR1, CDR2, and CDR3 sequences of SEQ ID NOs: 23, 24, and 25. In some embodiments, the VHH domain comprises the CDR1, CDR2, and CDR3 sequences of SEQ ID NOs: 28, 29, and 30. In some embodiments, the VHH domain comprises the CDR1, CDR2, and CDR3 sequences of SEQ ID NOs: 33, 34, and 35. In some embodiments, the VHH domain comprises the CDR1, CDR2, and CDR3 sequences of SEQ ID NOs: 38, 39, and 40.

In certain embodiments, under the Chothia definition, a Siglec-8 binding VHH domain comprises consensus hzG2 CDR1, CDR2, and CDR3 sequences set forth in SEQ ID NOs: 149, 150, and 151, respectively. In certain embodiments, the X₁ in SEQ ID NO: 151 is Q, S, or T. In certain embodiments, the X2 in SEQ ID NO: 151 is T. In various embodiments, the VHH domain comprises the CDR1 sequence of SEQ ID NO: 74, 75, or 76; a CDR2 sequence of SEQ ID NO: 80 or 81; and the CDR3 sequence of SEQ ID NO: 8. In some embodiments, the VHH domain comprises the CDR1, CDR2, and CDR3 sequences of SEQ ID NOs: 74, 80, and 8. In some embodiments, the VHH domain comprises the CDR1, CDR2, and CDR3 sequences of SEQ ID NOs: 75, 80, and 8. In some embodiments, the VHH domain comprises the CDR1, CDR2, and CDR3 sequences of SEQ ID NOs: 76, 80, and 8. In some embodiments, the VHH domain comprises the CDR1, CDR2, and CDR3 sequences of SEQ ID NOs: 76, 81, and 8.

In certain embodiments, under the Kabat definition, a Siglec-8 binding VHH domain comprises consensus hzG2 CDR1, CDR2, and CDR3 sequences set forth in SEQ ID NOs: 152, 153, and 154, respectively. In certain embodiments, the X₁ in SEQ ID NO: 154 is Q, S, or T. In certain embodiments, the X2 in SEQ ID NO: 154 is T. In various embodiments, the VHH domain comprises the CDR1 sequence of SEQ ID NO: 85, 86, or 87; a CDR2 sequence of SEQ ID NO: 92, 93, or 94; and the CDR3 sequence of SEQ ID NO: 8. In some embodiments, the VHH domain comprises the CDR1, CDR2, and CDR3 sequences of SEQ ID NOs: 85, 92, and 8. In some embodiments, the VHH domain comprises the CDR1, CDR2, and CDR3 sequences of SEQ ID NOs: 86, 92, and 8. In some embodiments, the VHH domain comprises the CDR1, CDR2, and CDR3 sequences of SEQ ID NOs: 87, 93, and 8. In some embodiments, the VHH domain comprises the CDR1, CDR2, and CDR3 sequences of SEQ ID NOs: 87, 92, and 8. In some embodiments, the VHH domain comprises the CDR1, CDR2, and CDR3 sequences of SEQ ID NOs: 87, 94, and 8.

In certain embodiments, under the Contact definition, a Siglec-8 binding VHH domain comprises consensus hzG2 CDR1, CDR2, and CDR3 sequences set forth in SEQ ID NOs: 155, 156, and 157, respectively. In certain embodiments, the X₁ in SEQ ID NO: 157 is Q, S, or T. In certain embodiments, the X2 in SEQ ID NO: 157 is T. In various embodiments, the VHH domain comprises the CDR1 sequence of SEQ ID NO: 98, 99, or 100; a CDR2 sequence of SEQ ID NO: 104 or 105; and the CDR3 sequence of SEQ ID NO: 110. In some embodiments, the VHH domain comprises the CDR1, CDR2, and CDR3 sequences of SEQ ID NOs: 98, 104, and 110. In some embodiments, the VHH domain comprises the CDR1, CDR2, and CDR3 sequences of SEQ ID NOs: 99, 104, and 110. In some embodiments, the VHH domain comprises the CDR1, CDR2, and CDR3 sequences of SEQ ID NOs: 100, 104, and 110. In some embodiments, the VHH domain comprises the CDR1, CDR2, and CDR3 sequences of SEQ ID NOs: 100, 105, and 110.

In certain embodiments, under the IMGT definition, a Siglec-8 binding VHH domain comprises consensus hzG2 CDR1, CDR2, and CDR3 sequences set forth in SEQ ID NOs: 158, 159, and 160, respectively. In certain embodiments, the X₁ in SEQ ID NO: 160 is Q, S, or T. In certain embodiments, the X2 in SEQ ID NO: 160 is T. In various embodiments, the VHH domain comprises the CDR1 sequence of SEQ ID NO: 114, 115, or 116; a CDR2 sequence of SEQ ID NO: 120 or 121; and the CDR3 sequence of SEQ ID NO: 126. In some embodiments, the VHH domain comprises the CDR1, CDR2, and CDR3 sequences of SEQ ID NOs: 114, 120, and 126. In some embodiments, the VHH domain comprises the CDR1, CDR2, and CDR3 sequences of SEQ ID NOs: 115, 120, and 126. In some embodiments, the VHH domain comprises the CDR1, CDR2, and CDR3 sequences of SEQ ID NOs: 116, 120, and 126. In some embodiments, the VHH domain comprises the CDR1, CDR2, and CDR3 sequences of SEQ ID NOs: 116, 121, and 126.

In some embodiments, the VHH domain comprises the CDR1, CDR2, and CDR3 sequences of SEQ ID NOs: 2, 3, and 4, respectively, under the AbM definition. In some embodiments, the VHH domain comprises the CDR1, CDR2, and CDR3 sequences of SEQ ID NOs: 52, 53, and 54, respectively, under the AbM definition. In some embodiments, the VHH domain comprises the CDR1, CDR2, and CDR3 sequences of SEQ ID NOs: 58, 59, and 60, respectively, under the AbM definition. In some embodiments, the VHH domain comprises the CDR1 and CDR2 sequences of SEQ ID NOs: 63 and 64, respectively, and a CDR3 comprising the amino acid sequence “GAY”, under the AbM definition. In various embodiments, the VHH domain is humanized.

In some embodiments, the VHH domain comprises the CDR1, CDR2, and CDR3 sequences of SEQ ID NOs: 74, 80, and 4, respectively, under the Chothia definition. In some embodiments, the VHH domain comprises the CDR1, CDR2, and CDR3 sequences of SEQ ID NOs: 77, 82, and 54, respectively, under the Chothia definition. In some embodiments, the VHH domain comprises the CDR1, CDR2, and CDR3 sequences of SEQ ID NOs: 78, 83, and 60, respectively, under the Chothia definition. In some embodiments, the VHH domain comprises the CDR1 and CDR2 sequences of SEQ ID NOs: 79 and 84, respectively, and a CDR3 comprising the amino acid sequence “GAY”, under the Chothia definition. In various embodiments, the VHH domain is humanized.

In some embodiments, the VHH domain comprises the CDR1, CDR2, and CDR3 sequences of SEQ ID NOs: 85, 91, and 4, respectively, under the Kabat definition. In some embodiments, the VHH domain comprises the CDR1, CDR2, and CDR3 sequences of SEQ ID NOs: 88, 95, and 54, respectively, under the Kabat definition. In some embodiments, the VHH domain comprises the CDR1, CDR2, and CDR3 sequences of SEQ ID NOs: 89, 96, and 60, respectively, under the Kabat definition. In some embodiments, the VHH domain comprises the CDR1 and CDR2 sequences of SEQ ID NOs: 90 and 97, respectively, and a CDR3 comprising the amino acid sequence “GAY”, under the Kabat definition. In various embodiments, the VHH domain is humanized.

In some embodiments, the VHH domain comprises the CDR1, CDR2, and CDR3 sequences of SEQ ID NOs: 98, 104, and 109, respectively, under the Contact definition. In some embodiments, the VHH domain comprises the CDR1, CDR2, and CDR3 sequences of SEQ ID NOs: 101, 106, and 111, respectively, under the Contact definition. In some embodiments, the VHH domain comprises the CDR1, CDR2, and CDR3 sequences of SEQ ID NOs: 102, 107, and 112, respectively, under the Contact definition. In some embodiments, the VHH domain comprises the CDR1, CDR2, and CDR3 sequences of SEQ ID NOs: 103, 108, and 113, respectively, under the Contact definition. In various embodiments, the VHH domain is humanized.

In some embodiments, the VHH domain comprises the CDR1, CDR2, and CDR3 sequences of SEQ ID NOs: 114, 120, and 125, respectively, under the IMGT definition. In some embodiments, the VHH domain comprises the CDR1, CDR2, and CDR3 sequences of SEQ ID NOs: 117, 122, and 127, respectively, under the IMGT definition. In some embodiments, the VHH domain comprises the CDR1, CDR2, and CDR3 sequences of SEQ ID NOs: 118, 123, and 128, respectively, under the IMGT definition. In some embodiments, the VHH domain comprises the CDR1, CDR2, and CDR3 sequences of SEQ ID NOs: 119, 124, and 129, respectively, under the IMGT definition. In various embodiments, the VHH domain is humanized.

In some embodiments, an antibody provided herein comprises one to three CDRs of a VHH domain selected from SEQ ID NOs: 1, 5, 11, 16, 21, 26, 31, 36, 49, 55, or 61. In some embodiments, an antibody provided herein comprises two to three CDRs of a VHH domain selected from SEQ ID NOs: 1, 5, 11, 16, 21, 26, 31, 36, 49, 55, or 61. In some embodiments, an antibody provided herein comprises three CDRs of a VHH domain selected from SEQ ID NOs: 1, 5, 11, 16, 21, 26, 31, 36, 49, 55, or 61. In some aspects, the CDRs are Kabat CDRs. In some aspects, the CDRs are Chothia CDRs. In some aspects, the CDRs are AbM CDRs. In some aspects, the CDRs are Contact CDRs. In some aspects, the CDRs are IMGT CDRs.

In some embodiments, the CDRs are CDRs having at least about 50%, 75%, 80%, 85%, 90%, or 95% identity with the CDRs provided herein. In some embodiments, the CDR1 is selected from SEQ ID NOs: 2, 6, 13, 18, 23, 28, 33, 38, 52, 58, 63, 74, 75, 76, 77, 78, 79, 85, 86, 87, 88, 89, 90, 98, 99, 100, 101, 102, 103, 114, 115, 116, 117, 118, or 119 with up to 1, 2, 3, 4, or 5 amino acid substitutions. In some embodiments, the CDR2 is selected from SEQ ID NOs: 3, 7, 14, 19, 24, 29, 34, 39, 53, 58, 64, 80, 81, 82, 83, 84, 91, 92, 93, 94, 95, 96, 97, 104, 105, 106, 107, 108, 120, 121, 122, 123, or 124, with up to 1, 2, 3, 4, 5, 6, 7, or 8 amino acid substitutions. In some embodiments, the CDR3 is selected from SEQ ID NOs: 4, 8, 15, 20, 30, 35, 40, 54, 60, “GAY”, 109, 110, 111, 112, 113, 125, 126, 127, 128, or 129 with up to 1, 2, 3, 4, 5, 6, 7, or 8 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, a VHH domain that binds Siglec-8 comprises a modification at Leu 11 within framework 1. For example, the VHH domain may comprise Leu11Glu (L11E), Leu11Asp (L11D), Leu11Arg (L11R) or Leu11Lys (L11K). In some embodiments, a VHH domain that binds Siglec-8 comprises a modified C-terminal sequence, such as TVKP (SEQ ID NO: 51), TVKPG (SEQ ID NO: 52), TVKPGG (SEQ ID NO: 50), TVRP (SEQ ID NO: 53), TVRPG (SEQ ID NO: 54), TVRPGG (SEQ ID NO: 55), TVEP (SEQ ID NO: 56), TVEPG (SEQ ID NO: 57), TVEPGG (SEQ ID NO: 58), TVDP (SEQ ID NO: 59), TVDPG (SEQ ID NO: 60) or TVDPGG (SEQ ID NO: 61). In some embodiments, the modified C-terminal sequence is LVTVKPGG (SEQ ID NO: 49). In some embodiments, a VHH domain that binds Siglec-8 comprises a modification at Leu 11 and modified C-terminal sequence. The L11E mutation and modified C-terminal sequence provide improved properties to the Siglec-8 binding polypeptide, such as reduced immunogenicity. See, e.g., WO 2016/118733.

In some such embodiments, the VHH domain comprises an amino acid sequence that is at least 85%, at least 90%, at least 95%, at least 96%, at least 97%, at least 98%, at least 99% identical to an amino acid sequence selected from SEQ ID NOs: 1, 5, 11, 16, 21, 26, 31, 36, 49, 61, 130-138, 140-148, 161, 163, and 164 (e.g., an amino acid sequence herein comprising the CDR1, CDR2, and CDR3 sequences of the VHH domain). In some embodiments, a VHH domain that binds Siglec-8 comprises an amino acid sequence selected from SEQ ID NOs: 1, 5, 11, 16, 21, 26, 31, 36, 49, 55, 61, 130-138, 140-148, 161, 163, and 164.

In various embodiments, a Siglec-8 binding polypeptide comprises one, two, three, or four VHH domains that bind Siglec-8. In various embodiments, a Siglec-8 binding polypeptide comprises two or three VHH domains that bind Siglec-8.

In some embodiment, a Siglec-8 binding polypeptide comprises two, three, or four VHH domains that bind Siglec-8 operably linked to each other via a peptide linker. A non-limiting example of a linker which may be used to link VHH domains is presented in SEQ ID NO: 69.

In some embodiments, a Siglec-8 binding polypeptide comprises at least one VHH domain described herein fused to an Fc region. In some embodiments, the Fc region comprises the sequence of SEQ ID NO: 44, 45, 65, 66, 67, or 68.

In some embodiment, a Siglec-8 binding polypeptide comprises at least one VHH domain described herein operably linked to an Fc region via a peptide linker. A non-limiting example of a linker which may be used to link a VHH domain and an Fc region is presented in SEQ ID NO: 70.

In some embodiments, a VHH domain that binds Siglec-8 is humanized. Humanized antibodies (such as sdAbs or VHH-containing polypeptides) are useful as therapeutic molecules because humanized antibodies reduce or eliminate the human immune response to non-human antibodies, which can result in an immune response to an antibody therapeutic, and decreased effectiveness of the therapeutic. Generally, a humanized antibody comprises one or more variable domains in which CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (for example, the antibody from which the CDR residues are derived), for example, to restore or improve antibody specificity or affinity.

Humanized antibodies and methods of making them are reviewed, for example, in Almagro and Fransson, (2008) Front. Biosci. 13: 1619-1633, and are further described, for example, in Riechmann et al., (1988) Nature 332:323-329; Queen et al., (1989) Proc. Natl Acad. Sci. USA 86: 10029-10033; U.S. Pat. Nos. 5,821,337, 7,527,791, 6,982,321, and 7,087,409; Kashmiri et al., (2005) Methods 36:25-34; Padlan, (1991) Mol. Immunol. 28:489-498 (describing “resurfacing”); Dall'Acqua et al., (2005) Methods 36:43-60 (describing “FR shuffling”); and Osbourn et al., (2005) Methods 36:61-68 and Klimka et al., (2000) Br. J. Cancer, 83:252-260 (describing the “guided selection” approach to FR shuffling).

Human framework regions that can be used for humanization include but are not limited to: framework regions selected using the “best-fit” method (see, for example, Sims et al. (1993) J. Immunol. 151:2296); framework regions derived from the consensus sequence of human antibodies of a particular subgroup of heavy chain variable regions (see, for example, Carter et al. (1992) Proc. Natl. Acad. Sci. USA, 89:4285; and Presta et al. (1993) J. Immunol, 151:2623); human mature (somatically mutated) framework regions or human germline framework regions (see, for example, Almagro and Fransson, (2008) Front. Biosci. 13:1619-1633); and framework regions derived from screening FR libraries (see, for example, Baca et al., (1997) J. Biol. Chem. 272: 10678-10684 and Rosok et al., (1996) J. Biol. Chem. 271:22611-22618). Typically, the FR regions of a VHH are replaced with human FR regions to make a humanized VHH. In some embodiments, certain FR residues of the human FR are replaced in order to improve one or more properties of the humanized VHH. VHH domains with such replaced residues are still referred to herein as “humanized.”

### Polypeptide and Protein Expression and Production

Nucleic acid molecules comprising polynucleotides that encode a Siglec-8 binding polypeptide or protein are provided. In some embodiments, the nucleic acid molecule may also encode a leader sequence that directs secretion of the Siglec-8 binding polypeptide or protein, which leader sequence is typically cleaved such that it is not present in the secreted polypeptide or protein. The leader sequence may be a native heavy chain (or VHH) leader sequence, or may be another heterologous leader sequence.

Nucleic acid molecules can be constructed using recombinant DNA techniques conventional in the art. In some embodiments, a nucleic acid molecule is an expression vector that is suitable for expression in a selected host cell.

Vectors comprising nucleic acids that encode the Siglec-8 binding polypeptides and proteins described herein are provided. Such vectors include, but are not limited to, DNA vectors, phage vectors, viral vectors, retroviral vectors, etc. In some embodiments, a vector is selected that is optimized for expression of polypeptides or proteins in a desired cell type, such as CHO or CHO-derived cells, or in NSO cells. Exemplary such vectors are described, for example, in Running Deer et al., Biotechnol. Prog. 20:880-889 (2004).

In some embodiments, a Siglec-8 binding polypeptide or protein may be expressed in prokaryotic cells, such as bacterial cells; or in eukaryotic cells, such as fungal cells (such as yeast), plant cells, insect cells, and mammalian cells. Such expression may be carried out, for example, according to procedures known in the art. Exemplary eukaryotic cells that may be used to express polypeptides and proteins include, but are not limited to, COS cells, including COS 7 cells; 293 cells, including 293-6E cells; CHO cells, including CHO-S, DG44. Lec13 CHO cells, and FUT8 CHO cells; PER.C6 ® cells (Crucell); and NSO cells. In some embodiments, the Siglec-8 binding polypeptides or proteins may be expressed in yeast. See, e.g., U.S. Publication No. US 2006/0270045 A1. In some embodiments, a particular eukaryotic host cell is selected based on its ability to make desired post-translational modifications to the polypeptide or protein. For example, in some embodiments, CHO cells produce polypeptides or proteins that have a higher level of sialylation than the same polypeptide or protein produced in HEK 293 cells.

Introduction of one or more nucleic acids (such as vectors) into a desired host cell may be accomplished by any method, including but not limited to, calcium phosphate transfection, DEAE-dextran mediated transfection, cationic lipid-mediated transfection, electroporation, transduction, infection, etc. Nonlimiting exemplary methods are described, for example, in Sambrook et al., Molecular Cloning, A Laboratory Manual, 3^{rd} ed. Cold Spring Harbor Laboratory Press (2001). Nucleic acids may be transiently or stably transfected in the desired host cells, according to any suitable method.

Host cells comprising any of the nucleic acids or vectors described herein are also provided. In some embodiments, a host cell that expresses a Siglec-8 binding polypeptide or protein described herein is provided. The Siglec-8 binding polypeptides or proteins expressed in host cells can be purified by any suitable method. Such methods include, but are not limited to, the use of affinity matrices or hydrophobic interaction chromatography. Suitable affinity ligands include the ROR1 ECD and agents that bind Fc regions. For example, a Protein A, Protein G, Protein A/G, or an antibody affinity column may be used to bind the Fc region and to purify a Siglec-8 binding polypeptide or protein that comprises an Fc region. Hydrophobic interactive chromatography, for example, a butyl or phenyl column, may also be suitable for purifying some polypeptides or proteins such as antibodies. Ion exchange chromatography (for example anion exchange chromatography and/or cation exchange chromatography) may also be suitable for purifying some polypeptides or proteins such as antibodies. Mixed-mode chromatography (for example reversed phase/anion exchange, reversed phase/cation exchange, hydrophilic interaction/anion exchange, hydrophilic interaction/cation exchange, etc.) may also be suitable for purifying some polypeptides or proteins such as antibodies. Many methods of purifying polypeptides or proteins are known in the art.

In some embodiments, the Siglec-8 binding polypeptide or protein is produced in a cell-free system. Nonlimiting exemplary cell-free systems are described, for example, in Sitaraman et al., Methods Mol. Biol. 498: 229-44 (2009); Spirin, Trends Biotechnol. 22: 538-45 (2004); Endo et al., Biotechnol. Adv. 21: 695-713 (2003).

In some embodiments, Siglec-8 binding polypeptides or proteins prepared by the methods described above are provided. In some embodiments, the Siglec-8 binding polypeptide or protein is prepared in a host cell. In some embodiments, the Siglec-8 binding polypeptide or protein is prepared in a cell-free system. In some embodiments, the Siglec-8 binding polypeptide or protein is purified. In some embodiments, a cell culture media comprising a Siglec-8 binding polypeptide or protein is provided.

In some embodiments, compositions comprising antibodies prepared by the methods described above are provided. In some embodiments, the composition comprises a Siglec-8 binding polypeptide or protein prepared in a host cell. In some embodiments, the composition comprises a Siglec-8 binding polypeptide or protein prepared in a cell-free system. In some embodiments, the composition comprises a purified Siglec-8 binding polypeptide or protein.

### Exemplary Methods of Treating Diseases Using Siglec-8 Binding Polypeptides or Proteins

In some embodiments, methods of treating disease in an individual comprising administering a Siglec-8 binding polypeptide or protein are provided. In some embodiments, methods for treating a disease associated with Siglec-8 (e.g., a disease associated with increased Siglec-8 expression on certain cells relative to normal, reference cells of the same type and/or in the same tissue). Such diseases include eosinophilic disorders, including eosinophilic cystitis, eosinophilic fasciitis, an eosinophilic gastrointestinal disorder, eosinophilic gastritis (EoG), eosinophilic enteritis, Churg-Strauss syndrome, hypereosinophilic syndrome (HES) (e.g., familial HES, idiopathic HES, primary (neoplastic) HES, secondary (reactive) HES, or lymphoid variant of HES), Gleich Syndrome, Eosinophilia myalgia syndrome (EMS), IgG4-related disease (IgG4-RD), eosinophilic leukemias (e.g., chronic eosinophilic leukemia), asthma with an eosinophilic phenotype, allergic bronchopulmonary aspergillosis (ABPA), chronic rhinosinusitis with nasal polyps (CRSwNP), or eosinophilic granulomatosis with polyangiitis (EGPA). Additional exemplary eosinophil disorders and related syndromes are described in Valent et al., Allergy (2023) 78(1):47-59. Such diseases also include mast cell disorders, including Systemic Mastocytosis, hereditary alpha tryptasemia (HAT), mast cell activation syndrome (MCAS), or general allergic conditions. The Systemic Mastocytosis (SM) can be advanced SM (e.g., Mast Cell Leukemia, aggressive SM, or SM with associated hematologic neoplasm (SM-AHN)), or non-advanced SM (e.g., indolent SM). In certain embodiments, the subject having SM comprises a D816V mutation in c-KIT. In certain embodiments, increased expression of CD25 and/or CD2 is observed on mast cells of the subject having SM. The general allergic conditions can be induced by allergies such as food allergies (such as, for example, peanut, sesame, milk, egg, fish, or tree nut allergy), environmental allergies (such as, for example, dust mite, pollen, mold, or latex allergy), allergies to companion animals (such as, for example, cat, dog, gird, horse, or cow allergy), and venom allergies (such as, for example, bee, wasp, or spider allergy). In some embodiments, methods for treating allergic disorders are provided. The method comprises administering to the individual an effective amount of an Siglec-8 binding polypeptide or protein provided herein. In some embodiments, the Siglec-8 binding polypeptide or protein induces eosinophil cell death in the presence of IL-5. In some embodiments, the Siglec-8 binding polypeptide or protein inhibits activation of mast cells. In some embodiments, the Siglec-8-binding polypeptide or protein inhibits an inflammatory response.

In some embodiments, the Siglec-8 binding polypeptide or protein is linked to an additional agent, such as an additional therapeutic agent, to form an immunoconjugate.

In some embodiments, the methods of treatment may be in humans or animals. In some embodiments, methods of treating humans are provided.

The Siglec-8 binding polypeptides and proteins can be administered as needed to subjects. Determination of the frequency of administration can be made by persons skilled in the art, such as an attending physician based on considerations of the condition being treated, age of the subject being treated, severity of the condition being treated, general state of health of the subject being treated and the like. In some embodiments, an effective dose of a Siglec-8 binding polypeptide or protein is administered to a subject one or more times. In some embodiments, an effective dose of a Siglec-8 binding polypeptide or protein is administered to the subject daily, semiweekly, weekly, every two weeks, once a month, etc. An effective dose of a Siglec-8 binding polypeptide or protein is administered to the subject at least once. In some embodiments, the effective dose of a Siglec-8 binding polypeptide or protein may be administered multiple times.

In some embodiments, pharmaceutical compositions are administered in an amount effective for treating a disease associated with Siglec-8. The therapeutically effective amount is typically dependent on the weight of the subject being treated, his or her physical or health condition, the extensiveness of the condition to be treated, or the age of the subject being treated.

In some embodiments, Siglec-8 binding polypeptides or proteins can be administered in vivo by various routes, including, but not limited to, intravenous, intra-arterial, parenteral, intraperitoneal or subcutaneous. The appropriate formulation and route of administration may be selected according to the intended application.

### Pharmaceutical Compositions

In some embodiments, compositions comprising Siglec-8 binding polypeptides or proteins are provided in formulations with a wide variety of pharmaceutically acceptable carriers (see, for example, Gennaro, Remington: The Science and Practice of Pharmacy with Facts and Comparisons: Drugfacts Plus, 20th ed. (2003); Ansel et al., Pharmaceutical Dosage Forms and Drug Delivery Systems, 7^{th} ed., Lippencott Williams and Wilkins (2004); Kibbe et al., Handbook of Pharmaceutical Excipients, 3^{rd} ed., Pharmaceutical Press (2000)). Various pharmaceutically acceptable carriers, which include vehicles, adjuvants, and diluents, are available. Moreover, various pharmaceutically acceptable auxiliary substances, such as pH adjusting and buffering agents, tonicity adjusting agents, stabilizers, wetting agents and the like, are also available.

### Combination Therapy

Siglec-8 binding polypeptides and proteins of the present disclosure can be administered alone or in combination with other modes of treatment, such as an additional therapeutic agent. The Siglec-8 binding polypeptide or protein can be provided before, substantially contemporaneous with, or after the one or more other modes of treatment (i.e., concurrently or sequentially).

### Nonlimiting Exemplary Methods of Diagnosis

In some embodiments, the methods described herein are useful for evaluating a subject and/or a specimen from a subject. In some embodiments, evaluation is one or more of diagnosis, prognosis, and/or response to treatment.

In some embodiments, the methods described herein comprise evaluating a presence, absence, or level of a protein or other molecule, for example, Siglec-8, histamine, β-tryptase or other allergic mediators. In some embodiments, the methods described herein comprise evaluating a presence, absence, or level of expression of a nucleic acid. The compositions described herein may be used for these measurements. For example, in some embodiments, the methods described herein comprise contacting a sample, serum, or cells from a sample with a therapeutic agent as described herein.

### Kits

Also provided are articles of manufacture and kits that include any of the Siglec-8 binding polypeptides and proteins as described herein, and suitable packaging. In some embodiments, the invention includes a kit with (i) a Siglec-8 binding polypeptide or protein, and (ii) instructions for using the kit to administer the Siglec-8 binding polypeptide or protein to an individual.

Suitable packaging for compositions, such as pharmaceutical compositions, comprising polypeptides or proteins described herein are known in the art, and include, for example, vials (e.g., sealed vials), vessels, ampules, bottles, jars, flexible packaging (e.g., sealed Mylar or plastic bags), and the like. These articles of manufacture may further be sterilized and/or sealed. Also provided are unit dosage forms comprising the compositions described herein. These unit dosage forms can be stored in a suitable packaging in single or multiple unit dosages and may also be further sterilized and sealed. Instructions supplied in the kits of the invention are typically written instructions on a label or package insert (e.g., a paper sheet included in the kit), but machine-readable instructions (e.g., instructions carried on a magnetic or optical storage disk) are also acceptable. The instructions relating to the use of the antibodies generally include information as to dosage, dosing schedule, and route of administration for the intended treatment or industrial use.

The containers may be unit doses, bulk packages (e.g., multi-dose packages) or sub-unit doses. For example, kits may also be provided that contain sufficient dosages of polypeptides or proteins disclosed herein to provide effective treatment for an individual for an extended period. Kits may also include multiple unit doses of polypeptides or proteins and instructions for use and packaged in quantities sufficient for storage and use in pharmacies, for example, hospital pharmacies and compounding pharmacies.

### EXAMPLES

The examples discussed below are intended to be purely exemplary of the invention and should not be considered to limit the invention in any way. The examples are not intended to represent that the experiments below are all or the only experiments performed. Efforts have been made to ensure accuracy with respect to numbers used (for example, amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1: Development of a Single Domain Antibody that Specifically Binds Human Siglec-8

Siglec-8 is an inhibitory receptor expressed on eosinophils, mast cells and basophils. Single domain antibodies targeting human Siglec8 were generated via immunization of llamas and alpacas with a recombinant version of the human Siglec-8 extracellular domain (ECD: amino acids 17-363 of human Siglec-8).

Following the development of specific anti-Siglec-8 antibody titers, llama/alpaca peripheral blood mononuclear cells (PBMCs) were isolated from 500 mL of blood from the immunized animals and total mRNA was isolated using the Qiagen RNeasy Maxi Kit and subsequently converted to first strand cDNA using Thermo Superscript IV Reverse Transcriptase and oligo-dT priming. Single domain antibody (sdAb) sequences were specifically amplified via PCR using cDNA as a template and cloned into a phage display vector pADL.

Phage libraries (3.7×10¹⁰ diversity) displaying llama derived sdAbs were enriched by cell panning of freestyle HEK 293 cells or CHO cells transfected to surface express the recombinant form of the Siglec8-ECD. DNA from the enriched Siglec8 phage library was transformed into BL21 competent cells which were plated and isolated into 96-well blocks. IPTG induction media allowed for sdAb secretion into the supernatant which was applied to Siglec-8 positive and negative cells. The cells were washed, treated with fluorophore labelled anti-Myc secondary antibody, and analyzed by 96-well flow cytometry.

Nucleic acid sequences encoding sdAbs that bound to Siglec-8 positive cells and not to Siglec-8 negative cells were cloned in-frame with a human Fc encoding region into mammalian expression vectors and expressed by transient transfection in HEK293 freestyle cells (293F cells) or CHO cells using polyethylenimine. Supernatant was collected after 3-7 days, secreted recombinant protein was purified by protein A chromatography, and concentration was calculated from the absorbance at 280 nm and extinction coefficient.

Binding of the anti-human Siglec-8 VHHs, B12, D1, E2, and G2, as a bivalent VHH-hIgG1-xELL-Fc (VHH of SEQ ID NO: 49, 55, 61, and 1, respectively linked to an Fc xELL via linker 2, these constructs are also designated cx8982, cx8983, cx8984, and cx10382, respectively) was determined by flow cytometry using CHO cells that had been transiently transfected with full-length human Siglec-8. Binding specificity was assessed using untransfected (UT) CHO cells as a control. For the binding assays, 50,000 Siglec-8 CHOs or UT CHOs were seeded in a 96-well plate and a titration of anti-Siglec8-VHH-xELL was added in FACS Buffer (PBS 1% BSA, 0.1% NaN3 pH 7.4) incubating for 30 minutes at 4° C. After washing, the plates were incubated with an A647-conjugated anti-human Fcγ specific secondary antibody (Jackson ImmunoResearch) diluted 1:2000 in FACS buffer for 30 minutes at 4° C. The plates were washed, and binding was determined by flow cytometry quantitating anti-human A647 mean fluorescent intensity using an Intellicyt iQue Plus.

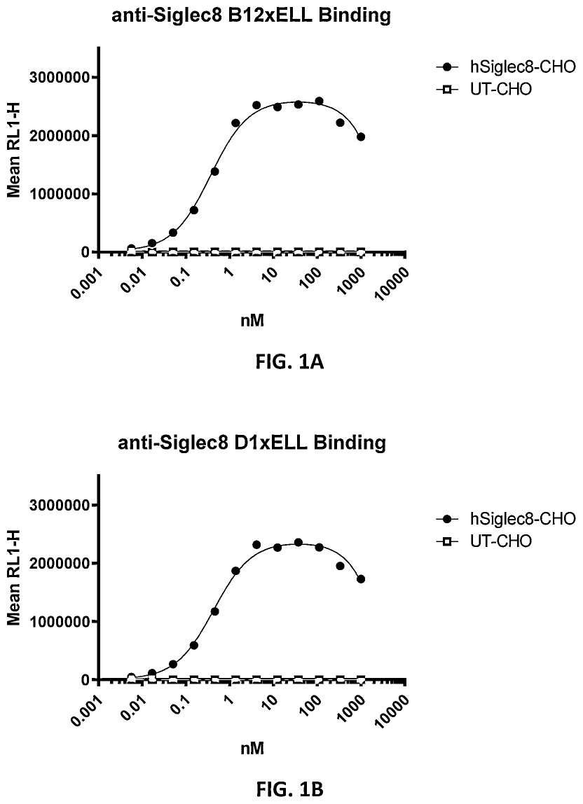

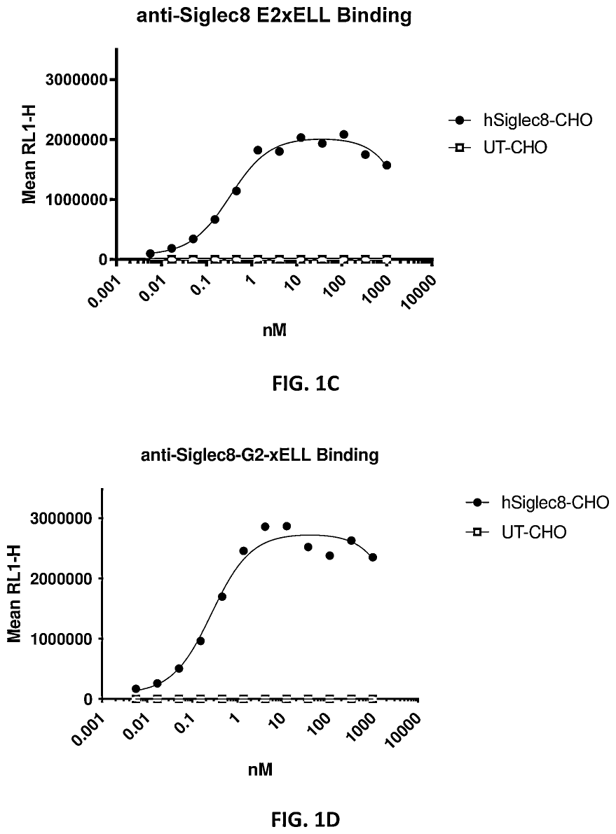

The binding of each anti-human Siglec-8 VHH-xELL Fc construct is shown in FIG. 1A-1D. Anti-Siglec8-B12-xELL (FIG. 1A) specifically binds to human Siglec-8 expressing CHO cells with a calculated affinity of 0.367 nM, anti-Siglec8-D1-xELL (FIG. 1B) specifically binds to human Siglec-8 expressing CHO cells with a calculated affinity of 0.427 nM, anti-Siglec8-E2-xELL (FIG. 1C) specifically binds to human Siglec-8 expressing CHO cells with a calculated affinity of 0.331 nM, and anti-Siglec8-G2-xELL (FIG. 1D) specifically binds to human Siglec-8 expressing CHO cells with a calculated affinity of 0.268 nM. The binding was specific to Siglec-8 as there was no detectable binding to untransfected CHO cells for any of the tested constructs.

### Example 2: Multivalent Anti-Siglec8-Fc Antibodies Bind to Human Siglec-8 Expressing Cells

To determine if increasing the valency of Siglec-8 antibodies could enhance agonist activity, the parental llama B12, D1 and E2 VHH sequences were used to generate tetravalent (Tet-B12 (cx8467; SEQ ID NO:50 linked to an Fc), Tet-D1 (cx8469; SEQ ID NO:56 linked to an Fc), and Tet-E2 (cx8463; SEQ ID NO:62 linked to an Fc)) and hexavalent (Hex-B12 (cx8466; SEQ ID NO:51 linked to an Fc), Hex-D1 (cx8523; SEQ ID NO:57 linked to an Fc), and Hex-E2 (cx8522; SEQ ID NO:165 linked to an Fc)), Siglec8-VHH-Fc antibodies linked to a reduced effector function xELL human Fc region via linker 2. The bivalent (Bi-B12 (cx8982), Bi-D1 (cx8983) and Bi-E2 (cx8984)), tetravalent, and hexavalent Siglec8-VHH-Fc antibodies were assessed by flow cytometry for binding to eosinophils (CD16⁻ CCR3⁺). Cells were isolated from whole blood by centrifugation, washed twice with 2% FBS PBS, and resuspended in 1×RBC lysis buffer for 10 minutes at room temperature (RT), washed once in 2% FBS PBS and treated with a second RBC lysis step and washed twice with 2% FBS. Cells were seeded at 200 k/well and resuspended in Human FC block for 10 minutes at RT. Cells were spun down and then resuspended in titrated test articles starting at 200 nM 1:4 and incubated for 30 minutes at 4° C. before being washed twice and resuspended in secondary mixture (2*; anti-CCR3 FITC, anti-human AF647, PI, anti-CD16 BV605) and incubated for 30 minutes at 4° C., washed twice, and bound antibodies were detected via flow cytometry (Novocyte).

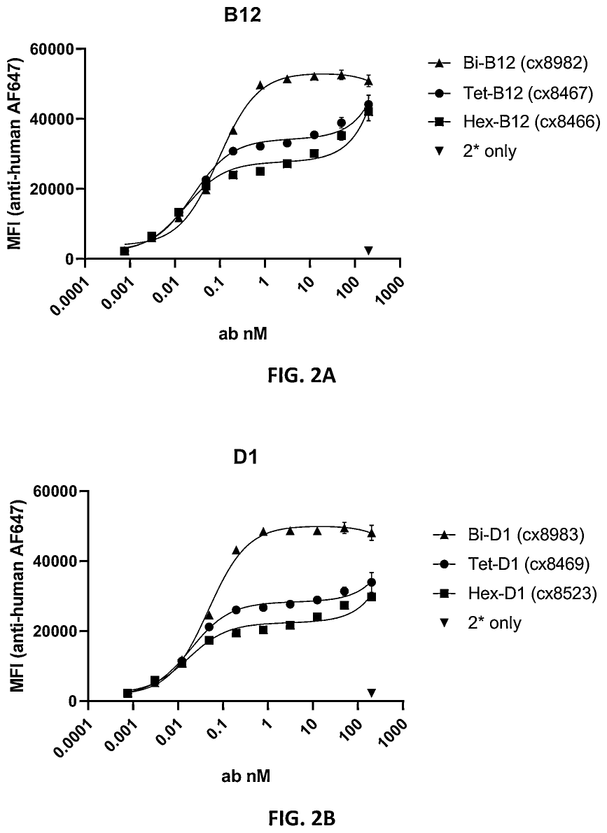

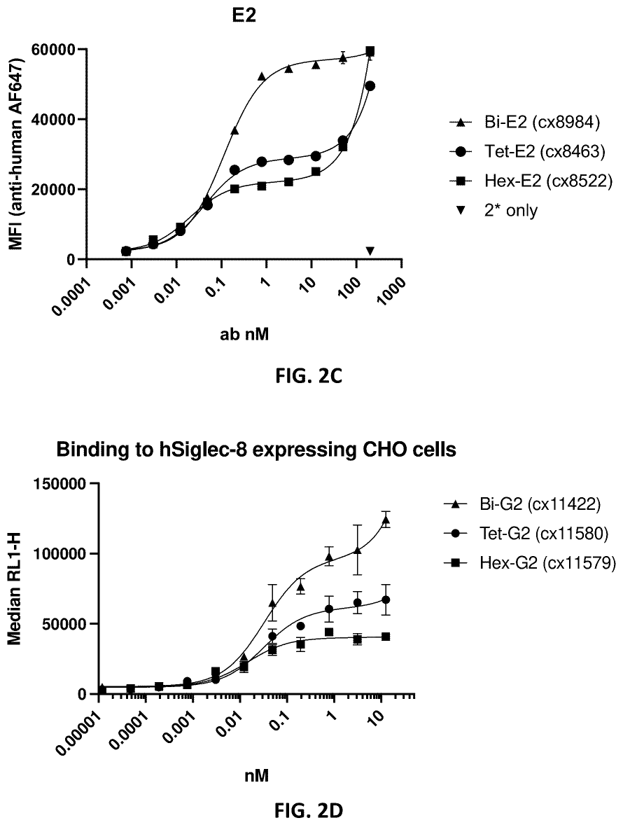

As shown in FIG. 2A-2C, bivalent, tetravalent, and hexavalent Siglec8-VHH-Fc antibodies B12 (FIG. 2A), D1 (FIG. 2B) and E2 (FIG. 2C) bound to eosinophils. The calculated affinity was similar between the bivalent, tetravalent, and hexavalent hzSiglec8-VHH-Fc antibodies. The observed Bmax was reduced with increasing valency as anticipated, due the fact that the Fc portion of the antibody was detected in this assay and the ratio of Fc regions to anti-Siglec-8 binding domains is inverse to valency. No binding was observed on other granulocyte populations (CD16⁺ CCR3⁻).

In additional studies, the parental llama G2 sequence was humanized and multivalent hzSiglec8-G2v14-Fc antibodies were cloned with a reduced effector function xELL human Fc. Binding of bivalent (cx11422; SEQ ID NO: 5 linked to an Fc xELL via linker 2), tetravalent (cx11580; SEQ ID NO: 9 linked to an Fc xELL via linker 2), and hexavalent (cx11579; SEQ ID NO: 10 linked to an Fc xELL via linker 2) hzSiglec8-G2v14-Fc antibodies were assessed by flow cytometry using CHO cells transfected with human Siglec-8. A titration series of the fusion proteins were incubated with the Siglec8-expressing CHO cells (5×10⁴ cells/well) for 30 minutes at 4° C. in FACS Buffer (PBS 1% BSA, 0.1% NaN3 pH 7.4) in 96 well plates. Following one 150 μL wash with FACS buffer, an APC-conjugated anti-human Fcγ specific secondary antibody (Jackson ImmunoResearch) was added, and cells were incubated for 30 minutes at 4° C. Following one additional wash in FACS buffer, bound antibodies were detected via flow cytometry (iQue Intellicyte).

As shown in FIG. 2D, bivalent, tetravalent, and hexavalent hzSiglec8-G2v14-Fc antibodies bound to CHO cells expressing human Siglec-8. The calculated affinity was similar between the bivalent, tetravalent, and hexavalent hzSiglec8-G2v14-Fc antibodies. The observed Bmax was reduced with increasing valency as anticipated, due the fact that the Fc portion of the antibody was detected in this assay and the ratio of Fc regions to anti-Siglec-8 binding domains is inverse to valency.

### Example 3: Increased Valency of Anti-Siglec-8 Antibodies Leads to Enhanced Eosinophil Killing Activity

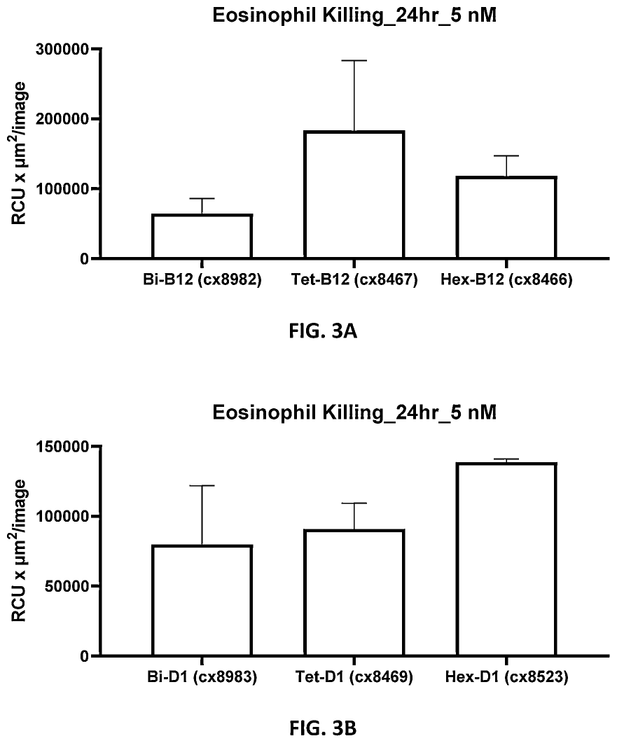

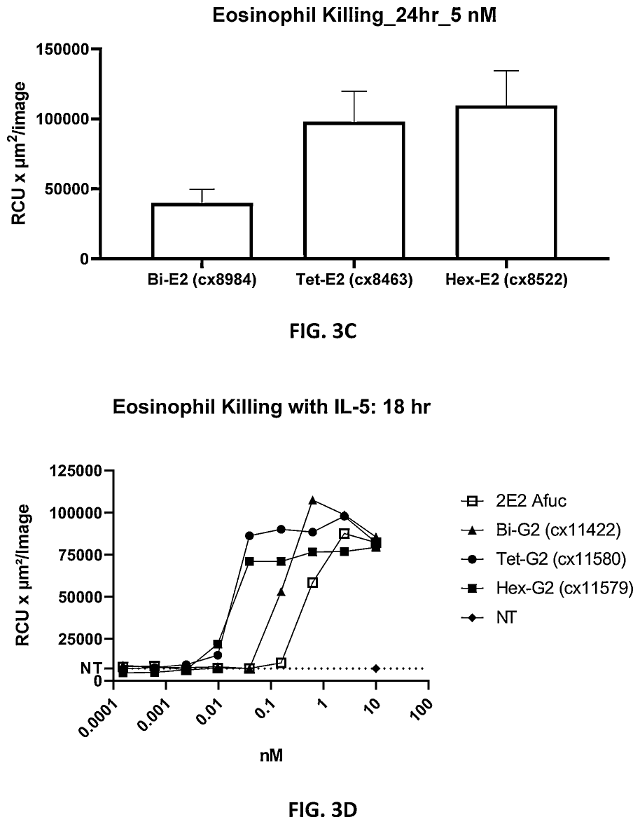

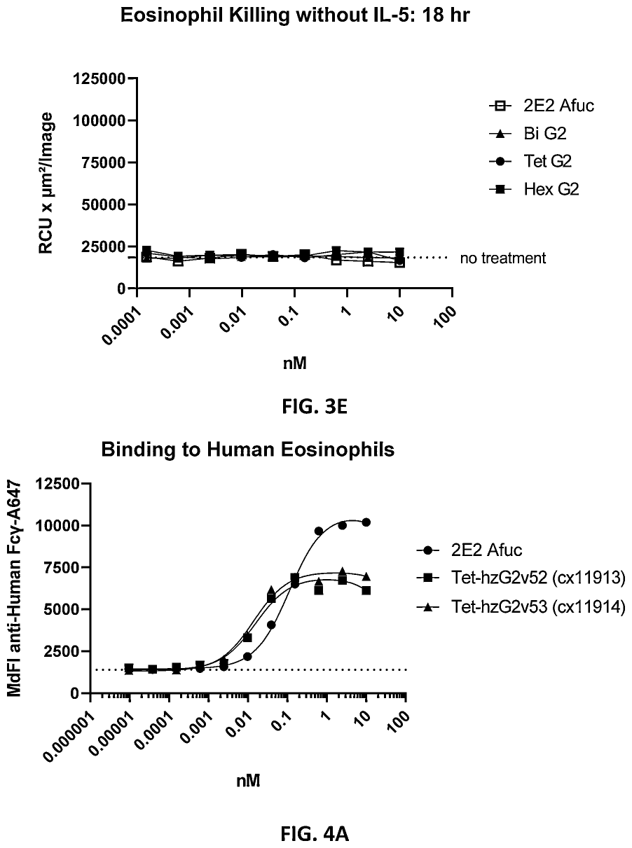

Siglec-8 agonism promotes the direct killing of eosinophils in the presence of IL-5. In order to assess the effect of valency on Siglec-8-mediated killing activity, eosinophils were isolated from healthy donor whole blood using the StemCell EasySep™ direct human eosinophil isolation kit, and treated with bivalent (Bi-B12 (cx8982), Bi-D1 (cx8983) and Bi-E2 (cx8984)), tetravalent (Tet-B12 (cx8467), Tet-D1 (cx8469), and Tet-E2 (cx8463)), and hexavalent (Hex-B12 (cx8466), Hex-D1 (cx8523), and Hex-E2 (cx8522)) Siglec8-VHH-Fc antibodies. The tested antibodies were titrated (3 point titration, 1:10 dilution) across the plate starting at 50 nM, the isolated eosinophils were resuspended to 160 k/mL in 10% FBS RPMI+Cytotox red and added at 80 k/well in a 48-well plate in the presence of IL-5. Incorporation of Cytotox Red was monitored over time using the Incucyte® cell analyzer and the total red object integrated intensity (RCU×μm²/image) was calculated at 24 hours using onboard software. The killing observed at 24 hours for cells treated with 5 nM of each tested antibody is plotted in FIG. 3A-3C. Increasing the valency from bivalent to tetravalent led to an increase in the potency of eosinophil killing for all of the Siglec8-VHH-Fc antibodies tested. Further increasing the valency to hexavalent resulted in similar or slightly increased potency relative to tetravalent valency.

In additional studies, the isolated eosinophils were added at 30,000 cells/well in a 96-well plate and treated with bivalent (cx11422), tetravalent (cx11580), or hexavalent (cx11579) hzSiglec8-G2v14-Fc antibodies, and incubated with the test articles titrated across the plate starting at 10 nM, in the presence of IL-5. The killing activity was compared to an internally derived afucosylated AK002 (lirentelimab) analog titrated in the same manner. To monitor eosinophil killing, Cytotox Red was added at a final concentration of 250 nM to each well. Incorporation of Cytotox Red was monitored over time using the Incucyte® cell analyzer and the total red object integrated intensity (RCU×μm²/image) was calculated at 18 hours using onboard software.

As shown in FIG. 3D, bivalent hzSiglec8-G2v14-Fc (cx11422) was more potent than the afucosylated AK002 (lirentelimab) analog antibody at potentiating the killing of eosinophils in the presence of IL-5. Increasing the valency of hzSiglec8-G2v14-Fc to tetravalent (cx11580) led to a dramatic increase in the potency of eosinophil killing resulting in an almost 30-fold improvement over the afucosylated AK002 analog antibody and a 10-fold increase compared to bivalent hzSiglec8-G2v14-Fc. Further increasing the valency to hexavalent (cx11579) did not result in more potent eosinophil killing relative to the tetravalent hzSiglec8-G2v14-Fc antibody but maintained superior killing activity compared to the afucosylated AK002 analog and bivalent hzSiglec8-G2v14-Fc. No killing was observed in the absence of IL-5 (FIG. 3E).

### Example 4: G2 VHH Sequence Optimization

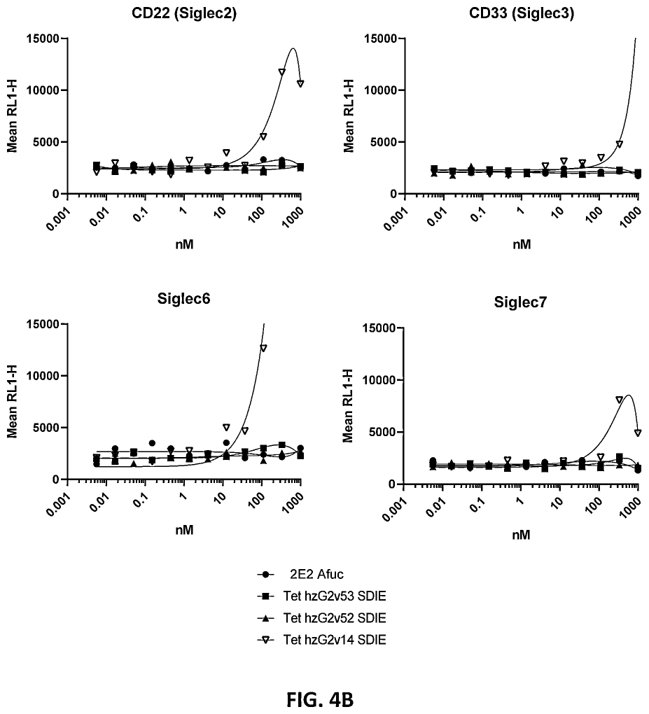

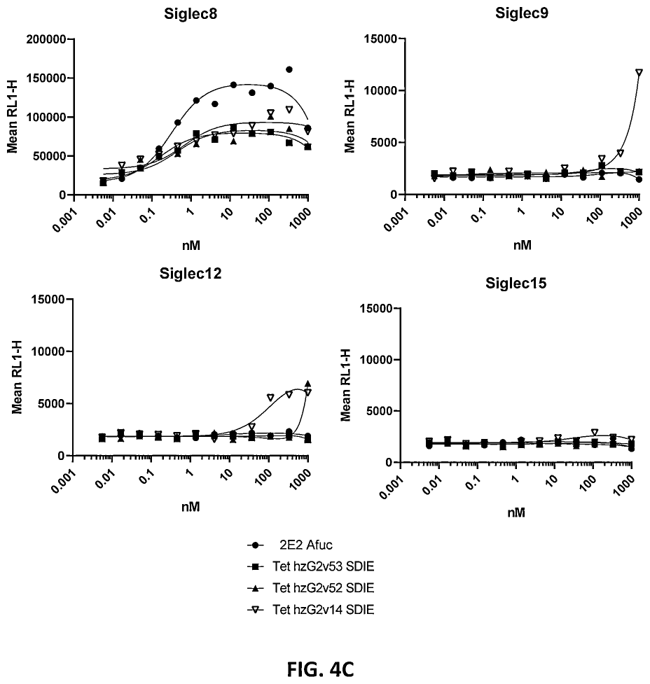

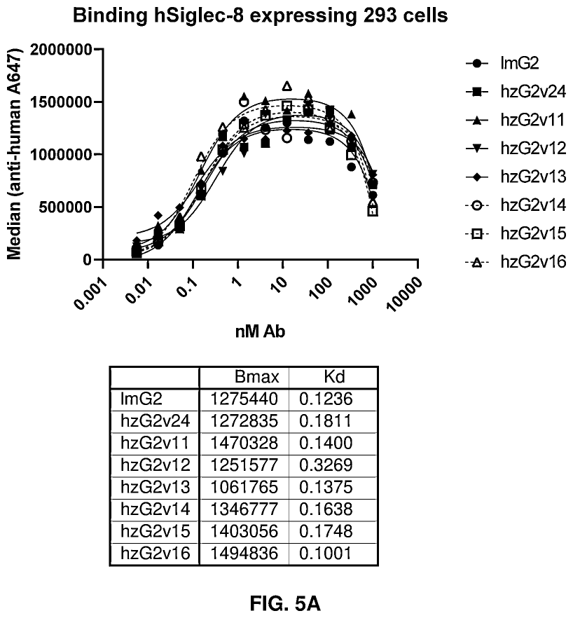

The anti-Siglec8 G2 VHH sequence hzG2v14 underwent further modification to minimize binding to siglec-2 (CD22) and other siglec proteins (see, below), reduce potential immunogenicity and optimize manufacturability. Humanized tetravalent Siglec-8 agonist antibodies comprising hzG2v14 (SEQ ID NO: 9), hzG2v47 (SEQ ID NO: 12), hzG2v51 (SEQ ID NO: 17), hzG2v52 (SEQ ID NO: 22), hzG2v53 (SEQ ID NO: 27), hzG2v54 (SEQ ID NO: 32) or hzG2v55 (SEQ ID NO: 37) were linked to an engineered Fc SDIE region (described further in Example 5) via linker 2. Binding to human Siglec-8 on eosinophils was evaluated and compared to that of the afucosylated anti-Siglec-8 2E2 (2E2 Afuc) reference antibody by flow cytometry. Eosinophils were isolated from healthy donor whole blood using the StemCell EasySep™ direct human eosinophil isolation kit, diluted in FACS buffer, added at 10 k/well to a 96-well assay plate and incubated at 4° C. for 20 minutes with a titration series (10-12 point titration, 1:4 dilution) starting at 10 nM of the test articles. Following one wash with FACS buffer, an Alexa Fluor® 647 AffiniPure Donkey Anti-Human Fcγ specific secondary antibody (Jackson ImmunoResearch) was added, and cells were incubated for 20 minutes at 4° C. Following one additional wash in FACS buffer, bound antibodies were detected via flow cytometry (NovoCyte 3000). Median fluorescent intensity was calculated using Novoexpress onboard software gating on SSC^{high}/CCR3⁺ cells. The dissociation constant (Kd) values from these studies are summarized in Table 3 and representative binding data for the tetravalent anti-Siglec8 antibodies comprising hzG2v52 (cx11913) and hzG2v53 (cx11914) is show in FIG. 4A. The pI of the VHH domains as determined using ExPasy are shown in Table 3, with a higher pI generally preferred for manufacturing.

TABLE 3
Humanized tetravalent Siglec-8 antibodies binding to eosinophiles

| Name | Kd (nM)* | pI |
|---|---|---|
| hzG2v14 | 0.00812 | 6.66 |
| hzG2v47 | 0.00888 | 5.39 |
| hzG2v51 | 0.119 | 6.67 |
| hzG2v52 | 0.012 | 5.42 |
| hzG2v53 | 0.013 | 6.66 |
| hzG2v54 | 0.036 | 6.59 |
| hzG2v55 | 0.010 | 6.58 |
| 2E2 Afuc | 0.112 | |

*determined from 1-3 experiments

As show in FIG. 4A, the tetravalent anti-Siglec8 antibodies cx11913 and cx11914 bound to the CCR3⁺ eosinophil population in a titration dependent manner. Both cx11913 and cx11914 bound with approximately 8-fold higher affinity than the 2E2 Afuc comparator antibody. As expected, the Bmax of cx11913 and cx11914 were both reduced compared to 2E2 Afuc due to fewer available Fc receptors being bound with the tetravalent antibodies.

This data demonstrates high affinity binding of the further modified humanized tetravalent anti-Siglec-8 antibodies comprising hzG2v47, hzG2v52, hzG2v53, hzG2v54 and hzG2v55 to the target human eosinophil population. In ELISA assays, the tetravalent anti-Siglec8 antibody comprising hzG2v47 exhibited some binding to recombinant Siglec-2 (CD22). The relative cross reactivity to Siglec-2 was:
hzG2v14>hzG2v47≈hzG2v54>hzG2v52≈hzG2v53≈hzG2v55.

The binding of the tetravalent Siglec-8 agonist antibodies cx11913 and cx11914, cx11769 (hzG2v14-Fc SDIE), and 2E2 Afuc to a panel of human siglec proteins transiently expressed on CHO cells was also evaluated by flow cytometry. CHO cells were transiently transfected (by PEI transfection in BalanCD Transfectory CHO cell culture media (Irvine Scientific)) with a panel of plasmids encoding the full-length extra cellular domain of a human Siglec-2, 3, 6, 7, 8, 9, 12, or 15 linked to a transmembrane protein for cell surface expression with co-expression of either citrine or GFP in the same vector. The next day a titration series (twelve point, 1:3 dilution) starting at 1 μM of the test articles was incubated with the Siglec-expressing cell lines (50 k cells/well) for 20 minutes at 4° C. in FACS Buffer (PBS 1% BSA, 0.1% NaN3 pH 7.4) in 96 well plates. Following one wash with FACS buffer, an Alexa Fluor® 647 AffiniPure Donkey Anti-Human Fcγ specific secondary antibody (Jackson ImmunoResearch) was added, and cells were incubated for 20 minutes at 4° C. Following one additional wash in FACS buffer, bound antibodies were detected via flow cytometry (iQue Intellicyte). Mean fluorescent intensity was calculated using iQue onboard software gating on GFP+ or citrine+ cells.

As shown in FIG. 4B-4C, the tetravalent anti-human Siglec-8 antibodies cx11913 (comprising hzG2v52) and cx11914 (hzG2v53) bound with high affinity to Siglec-8 (FIG. 4C, top left), but none of the other siglec proteins. At the highest 1 μM concentration tested, cx11913 bound to Siglec-12 (FIG. 4C, lower left), but did not show binding at any other concentrations. Both constructs demonstrated similar siglec specificity profiles to the 2E2 Afuc comparator antibody. In contrast, at certain higher concentrations tested, the anti-human Siglec-8 antibody cx11769 (comprising hzG2v14) exhibited cross reactivity with Siglec-2 as well as several other siglec proteins (FIGS. 4B-4C).

### Example 5: Assessment of Additional Humanized G2 VHH Constructs

The llama anti-Siglec8-G2 VHH domain (1mG2) was humanized to generate additional variants, namely, Siglec8 hzG2v11, Siglec8 hzG2v12, Siglec8 hzG2v13, Siglec8 hzG2v15, Siglec8 hzG2v16, Siglec8 hzG2v17, Siglec8 hzG2v18, Siglec8 hzG2v19, Siglec8 hzG2v20, Siglec8 hzG2v21, Siglec8 hzG2v22, Siglec8 hzG2v24, Siglec8 hzG2v25, Siglec8 hzG2v26, Siglec8 hzG2v28, Siglec8 hzG2v29, Siglec8 hzG2v30, Siglec8 hzG2v31, Siglec8 hzG2v32, Siglec8 hzG2v33, Siglec8 hzG2v2.1, Siglec8 hzG2v5.1, and Siglec8 hzG2v6.1, having the amino acid sequences set forth in SEQ ID NOs: 130-148 and 161-164. The substitution for asparagine (N) at position 2 or for serine (S) at position 3 of AbM CDR3 in hzG2v11-hzG2v14 was designed to eliminate a potential deamidation site.

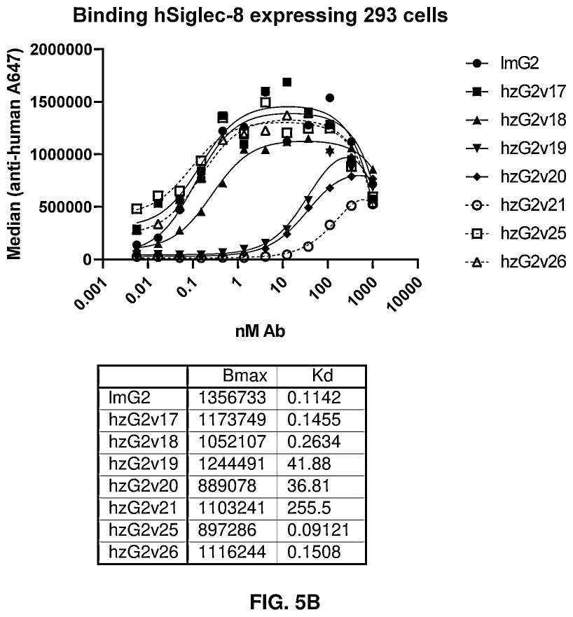

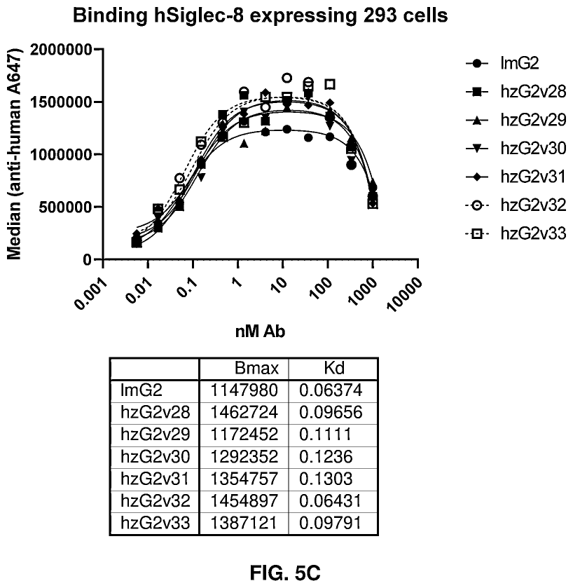

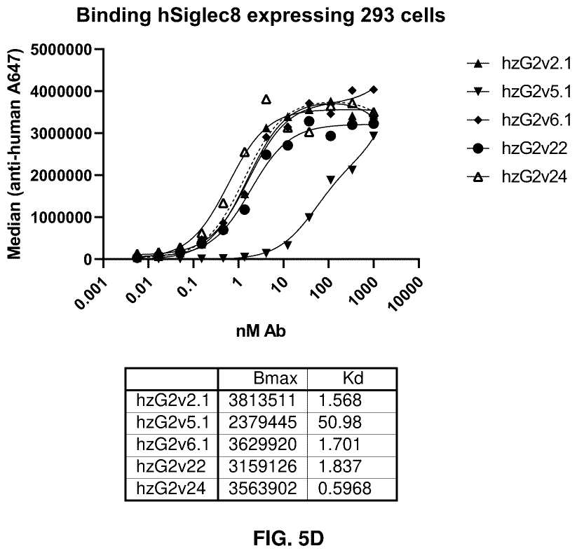

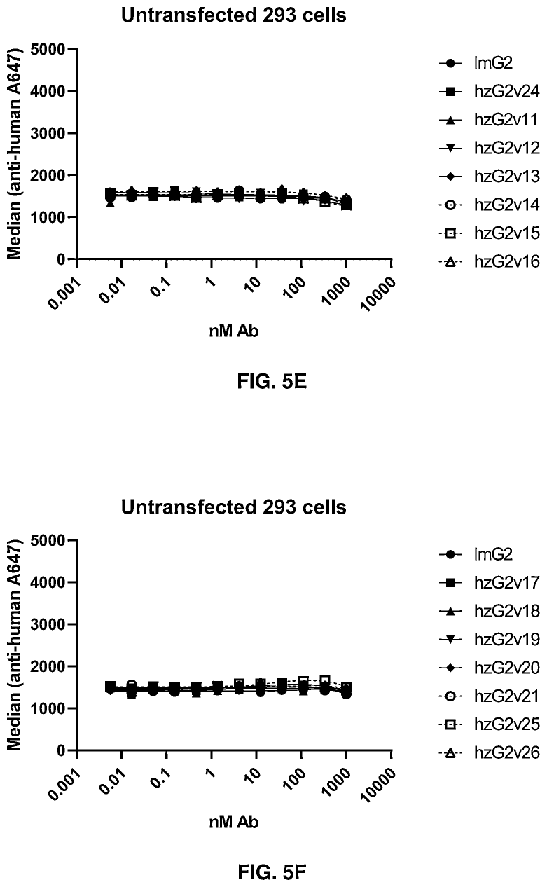

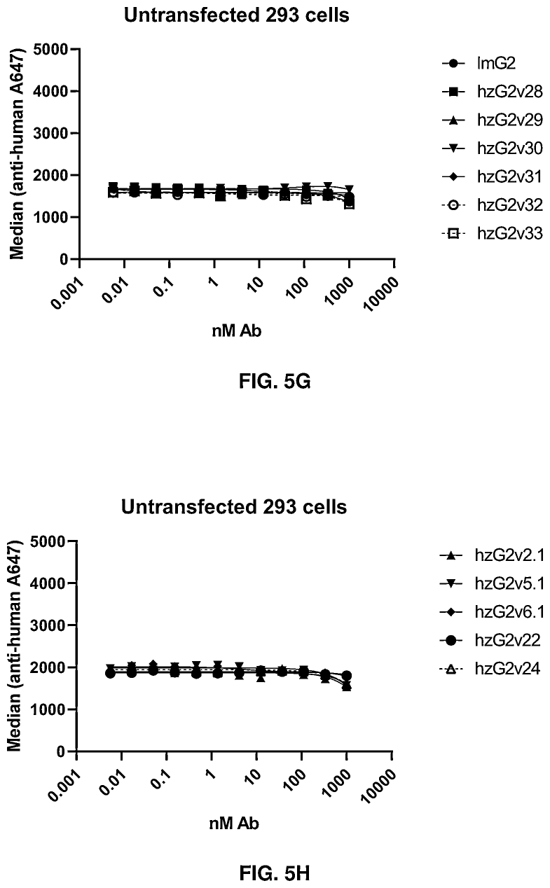

The binding of the 1mG2 and humanized versions (further including Siglec8 hzG2v14), formatted as monomeric VHH-hIgG1 fusions proteins using a non-dimerizing human IgG1 Fc variant region lacking a hinge Fc NNT, was assessed by flow cytometry using 293 cells that had been transiently transfected with full-length human Siglec-8 and detected with an anti-human AF647 secondary antibody. Binding specificity was assessed using untransfected 293 cells as a control. For the binding assays, Siglec-8 293 cells or untransformed 293 cells were seeded in a 96 well U-bottom plate with 50 k cells/well in FACS buffer and incubated at 4° C. with a titration of anti-Siglec8-VHH-NNT for 30 minutes. After washing, the plates were incubated with an A647-conjugated anti-human Fcγ specific secondary antibody diluted 1:10,000 in FACS buffer for 20 minutes at 4° C. The plates were washed, and binding was determined by flow cytometry quantitating anti-human A647 median fluorescent intensity. The maximum specific binding (Bmax) and affinity (Kd) for was determined from the flow cytometry binding data using a nonlinear fit model. The binding curves for each plate are presented in FIGS. 5A-5H.

Humanized versions of G2 had good expression and, as shown in FIGS. 5A-5D, almost all variants bound Siglec-8 with similar affinity as the parental clone, with the exception of hzG2v19-hzG2v21 and Siglec8 hzG2v5.1. No binding was observed on non-transfected cells (FIGS. 5E-5H).

### Example 6: Enhanced Antibody-Dependent Cellular Cytotoxicity (ADCC) Activity of Anti-Siglec-8 Antibodies Leads to Enhanced Eosinophil Killing Activity

As demonstrated above, bivalent, tetravalent and hexavalent anti-Siglec-8 antibodies can directly kill eosinophils in the presence of inflammatory mediators such as IL-5, with the tetravalent and hexavalent exhibiting higher relative potency than bivalent antibodies. However, the killing activity is eliminated in the absence of IL-5. To assess the effect of the Fc region on Siglec-8-mediated killing activity tetravalent anti-Siglec8-VHH-Fc antibodies comprising the humanized G2 VHH, hzG2v53, and either a wild-type human IgG1 Fc region (hIgG1) (cx12532; SEQ ID NO: 27 linked to the Fc (see, SEQ ID NO: 72)), or an engineered IgG1 Fc region with mutations at two residues, S239D and I332E (SDIE), to improve CD16 binding and subsequent antibody-dependent cell-mediated cytotoxicity (ADCC) activity (cx11914; SEQ ID NO: 27 linked to the Fc (see, SEQ ID NO: 71)), were generated. In addition, a bivalent anti-Siglec8-VHH-Fc antibody comprising hzG2v53 and the SDIE Fc region (cx12562; SEQ ID NO: 26 linked to the Fc (see, SEQ ID NO: 73) was generated. The hzG2v53 based tetravalent anti-Siglec8-VHH-Fc antibodies were evaluated in a direct eosinophil killing assay in the presence of IL-5. Eosinophils were isolated from healthy donor whole blood using the StemCell EasySep™ direct human eosinophil isolation kit, and added at 30,000 cells/well, in 10% FBS RPMI in the presence of IL-5 (10 ng/mL final) and incubated with a titration series (11 point titration, 1:4 dilution) of the tested antibodies starting at 100 nM. The killing activity was compared to an internally derived afucosylated AK002 (lirentelimab) analog titrated in the same manner. To monitor eosinophil killing, Cytotox Red was added at a final concentration of 250 nM to each well. Incorporation of Cytotox Red was monitored over time using the Incucyte® cell analyzer and the total red object integrated intensity (RCU×μm²/image) was calculated at 18 hours using onboard software.

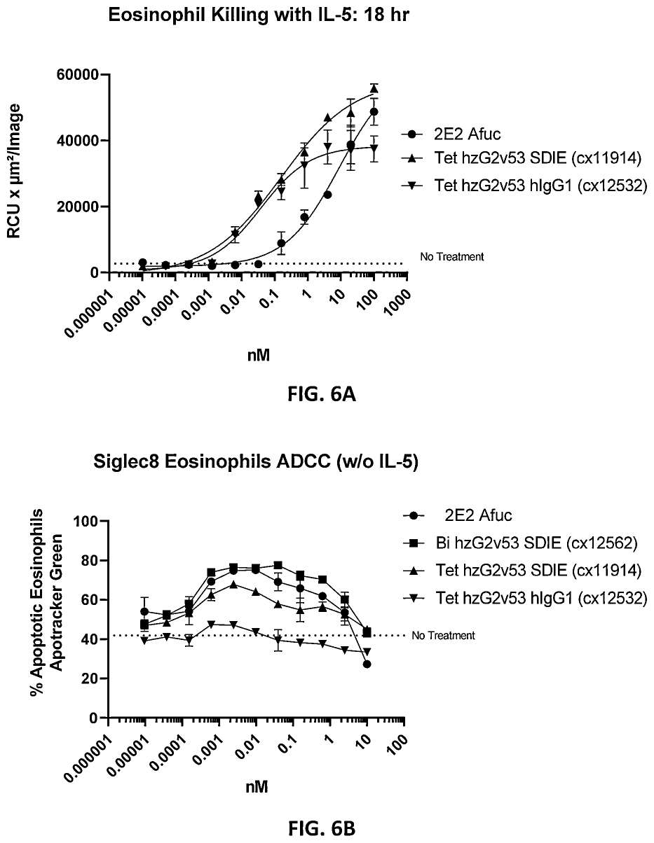

As shown in FIG. 6A, treatment with humanized tetravalent anti-Siglec-8 antibody constructs showed the highest potency in the presence of IL-5, demonstrating that the Fc modifications did not significantly impact direct eosinophil killing activity in the presence of IL-5.

The hzG2v53 based tetra- and bivalent anti-Siglec8-VHH-Fc antibodies were also evaluated for their ability to promote the ADCC of primary human eosinophils in the absence of IL-5 along with the afucosylated AK002 (lirentelimab) analog. Eosinophils were isolated as described above, and autologous NK cells were isolated from enriched PBMCs using the EasySep™ human NK cell enrichment kit. Antibodies were titrated in 96-well plates (11 point titration 1:4 dilution). Isolated NK cells (50,000 cells/well) and eosinophils (10,000 cells/well) were plated (5:1 Effector:Target ratio), in 100 μL 10% FBS RPMI+IL-2 (2 ng/mL final) for NK cell stimulation, no IL-5 was added. The next day, the cells were washed, and apoptotic target cells were labeled using Apotracker Green (Biolegend) for 20 minutes. Next, the cells were washed, resuspended in FACS buffer, and analyzed on a NovoCyte 3000 flow cytometer (ACEA Biosciences, Inc.).

As shown in FIG. 6B treatment with tet-hzG2v53-SDIE led to a dose-dependent increase in eosinophil killing, even in the absence of IL-5. The optimal range for ADCC activity was between 1-100 pM with a gradual decline at higher concentrations. The SDIE Fc modifications dramatically improved ADCC activity compared to the tet-hzG2v53-IgG1 with a wild type effector enabled Fc. Although, the tet-hzG2v53-SDIE antibody exhibited a small decrease in maximal ADCC killing activity compared to the of bi-hzG2v53-SDIE and 2E2 Afuc antibodies, as demonstrated above, the tetravalent format exhibited higher relative potency in direct killing assays.

The disclosure may be embodied in other specific forms without departing from the spirit or essential characteristics thereof. The foregoing embodiments are therefore to be considered in all respects illustrative rather than limiting of the disclosure. Scope of the disclosure is thus indicated by the appended claims rather than by the foregoing description, and all changes that come within the meaning and range of equivalency of the claims are therefore intended to be embraced herein.

Table of Certain Sequences

| SEQ | | |
|---|---|---|
| ID | | |
| NO | Description | Sequence |
| 1 | Siglec8-G2 VHH | QVTLKESGGGLVRIGGSLRLSCAASGSISIFSMYVVAWYRQAPGKQREWVA |
| | | FIQRGGTANYADSAKGRFTISRDNAKNTVYLQMASLKPDDTAVYYCNYINS |
| | | DNEIGGPWGQGTQVTVKP |
| 2 | Siglec8-G2 | GSISIFSMYVVA |
| | CDR1 | |
| 3 | Siglec8-G2 | FIQRGGTAN |
| | CDR2 | |
| 4 | Siglec8-G2 | INSDNEIGGP |
| | CDR3 | |
| 5 | hzG2v14 VHH | EVQLLESGGGEVQPGGSLRLSCAASGSISIFSMYVVAWYRQAPGKEREWVA |
| | | FIQRGGTANYAESAKGRFTISRDNAKNTVYLQMSSLRAEDTAVYYCNYINT |
| | | DNEIGGPWGQGTLVTVKP |
| 6 | hzG2v14 CDR1 | GSISIFSMYVVA |
| 7 | hzG2v14 CDR2 | FIQRGGTAN |
| 8 | hzG2v14 CDR3 | INTDNEIGGP |
| 9 | Siglec8-Tet- | EVQLLESGGGEVQPGGSLRLSCAASGSISIFSMYVVAWYRQAPGKEREWVA |
| | hzG2v14 VHH | FIQRGGTANYAESAKGRFTISRDNAKNTVYLQMSSLRAEDTAVYYCNYINT |
| | | DNEIGGPWGQGTLVTVKPGGSGGSEVQLLESGGGEVQPGGSLRLSCAASGS |
| | | ISIFSMYVVAWYRQAPGKEREWVAFIQRGGTANYAESAKGRFTISRDNAKN |
| | | TVYLQMSSLRAEDTAVYYCNYINTDNEIGGPWGQGTLVTVKP |
| 10 | Siglec8-Hex- | EVQLLESGGGEVQPGGSLRLSCAASGSISIFSMYVVAWYRQAPGKEREWVA |
| | hzG2v14 VHH | FIQRGGTANYAESAKGRFTISRDNAKNTVYLQMSSLRAEDTAVYYCNYINT |
| | | DNEIGGPWGQGTLVTVKPGGSGGSEVQLLESGGGEVQPGGSLRLSCAASGS |
| | | ISIFSMYVVAWYRQAPGKEREWVAFIQRGGTANYAESAKGRFTISRDNAKN |
| | | TVYLQMSSLRAEDTAVYYCNYINTDNEIGGPWGQGTLVTVKPGGSGGSEVQ |
| | | LLESGGGEVQPGGSLRLSCAASGSISIFSMYVVAWYRQAPGKEREWVAFIQ |
| | | RGGTANYAESAKGRFTISRDNAKNTVYLQMSSLRAEDTAVYYCNYINTDNE |
| | | IGGPWGQGTLVTVKP |
| 11 | hzG2v47 VHH | EVQLLESGGGEVQPGGSLRLSCAASGSISIFSDYVVAWYRQAPGKEREWVA |
| | | FIQRGGTANYAESAKGRFTISRDNAKNTVYLQMSSLRAEDTAVYYCNYINT |
| | | DNEIGGPWGQGTLVTVRP |
| 12 | Siglec8-Tet- | EVQLLESGGGEVQPGGSLRLSCAASGSISIFSDYVVAWYRQAPGKEREWVA |
| | hzG2v47 VHH | FIQRGGTANYAESAKGRFTISRDNAKNTVYLQMSSLRAEDTAVYYCNYINT |
| | | DNEIGGPWGQGTLVTVRPGGSGGSEVQLLESGGGEVQPGGSLRLSCAASGS |
| | | ISIFSDYVVAWYRQAPGKEREWVAFIQRGGTANYAESAKGRFTISRDNAKN |
| | | TVYLQMSSLRAEDTAVYYCNYINTDNEIGGPWGQGTLVTVRP |
| 13 | hzG2v47 CDR1 | GSISIFSDYVVA |
| 14 | hzG2v47 CDR2 | FIQRGGTAN |
| 15 | hzG2v47 CDR3 | INTDNEIGGP |
| 16 | hzG2v51 VHH | EVQLLESGGGEVQPGGSLRLSCAASGSISIFSKYVVAWYRQAPGKEREWVA |
| | | FIQRGGTANYAESAKDRFTISRDNAKNTVYLQMSSLRAEDTAVYYCNYINT |
| | | DNEIGGPWGQGTLVTVRP |
| 17 | Siglec8-Tet- | EVQLLESGGGEVQPGGSLRLSCAASGSISIFSKYVVAWYRQAPGKEREWVA |
| | hzG2v51 VHH | FIQRGGTANYAESAKDRFTISRDNAKNTVYLQMSSLRAEDTAVYYCNYINT |
| | | DNEIGGPWGQGTLVTVRPGGSGGSEVQLLESGGGEVQPGGSLRLSCAASGS |
| | | ISIFSKYVVAWYRQAPGKEREWVAFIQRGGTANYAESAKDRFTISRDNAKN |
| | | TVYLQMSSLRAEDTAVYYCNYINTDNEIGGPWGQGTLVTVRP |
| 18 | hzG2v51 CDR1 | GSISIFSKYVVA |
| 19 | hzG2v51 CDR2 | FIQRGGTAN |
| 20 | hzG2v51 CDR3 | INTDNEIGGP |
| 21 | hzG2v52 VHH | EVQLLESGGGEVQPGGSLRLSCAASGSISIFSKYVVAWYRQAPGKEREWVA |
| | | FIQRGGTANYAESAKGRFTISRDNAKNTVYLQMSSLRAEDTAVYYCNYINT |
| | | DNEIGGPWGQGTLVTVEP |
| 22 | Siglec8-Tet- | EVQLLESGGGEVQPGGSLRLSCAASGSISIFSKYVVAWYRQAPGKEREWVA |
| | hzG2v52 VHH | FIQRGGTANYAESAKGRFTISRDNAKNTVYLQMSSLRAEDTAVYYCNYINT |
| | | DNEIGGPWGQGTLVTVEPGGSGGSEVQLLESGGGEVQPGGSLRLSCAASGS |
| | | ISIFSKYVVAWYRQAPGKEREWVAFIQRGGTANYAESAKGRFTISRDNAKN |
| | | TVYLQMSSLRAEDTAVYYCNYINTDNEIGGPWGQGTLVTVEP |
| 23 | hzG2v52 CDR1 | GSISIFSKYVVA |
| 24 | hzG2v52 CDR2 | FIQRGGTAN |
| 25 | hzG2v52 CDR3 | INTDNEIGGP |
| 26 | hzG2v53 VHH | EVQLLESGGGEVQPGGSLRLSCAASGSISIFSKYVVAWYRQAPGKEREWVA |
| | | FIQSGGTANYAESAKGRFTISRDNAKNTVYLQMSSLRAEDTAVYYCNYINT |
| | | DNEIGGPWGQGTLVTVRP |
| 27 | Siglec8-Tet- | EVQLLESGGGEVQPGGSLRLSCAASGSISIFSKYVVAWYRQAPGKEREWVA |
| | hzG2v53 VHH | FIQSGGTANYAESAKGRFTISRDNAKNTVYLQMSSLRAEDTAVYYCNYINT |
| | | DNEIGGPWGQGTLVTVRPGGSGGSEVQLLESGGGEVQPGGSLRLSCAASGS |
| | | ISIFSKYVVAWYRQAPGKEREWVAFIQSGGTANYAESAKGRFTISRDNAKN |
| | | TVYLQMSSLRAEDTAVYYCNYINTDNEIGGPWGQGTLVTVRP |
| 28 | hzG2v53 CDR1 | GSISIFSKYVVA |
| 29 | hzG2v53 CDR2 | FIQSGGTAN |
| 30 | hzG2v53 CDR3 | INTDNEIGGP |
| 31 | hzG2v54 VHH | QVQLLESGGGEVQPGGSLRLSCAASGSISIFSKYVVAWYRQAPGKEREWVA |
| | | FIQRGGTANYAESAKGRFTISRDNAKNTVYLQMSSLRAEDTAVYYCNYINT |
| | | DNEIGGPWGQGTLVTVEP |
| 32 | Siglec8-Tet- | QVQLLESGGGEVQPGGSLRLSCAASGSISIFSKYVVAWYRQAPGKEREWVA |
| | hzG2v54 VHH | FIQRGGTANYAESAKGRFTISRDNAKNTVYLQMSSLRAEDTAVYYCNYINT |
| | | DNEIGGPWGQGTLVTVEPGGSGGSQVQLLESGGGEVQPGGSLRLSCAASGS |
| | | ISIFSKYVVAWYRQAPGKEREWVAFIQRGGTANYAESAKGRFTISRDNAKN |
| | | TVYLQMSSLRAEDTAVYYCNYINTDNEIGGPWGQGTLVTVEP |
| 33 | hzG2v54 CDR1 | GSISIFSKYVVA |
| 34 | hzG2v54 CDR2 | FIQRGGTAN |
| 35 | hzG2v54 CDR3 | INTDNEIGGP |
| 36 | hzG2v55 VHH | QVQLLESGGGEVQPGGSLRLSCAASGSISIFSDYVVAWYRQAPGKEREWVA |
| | | FIQRGGTANYAESAKGRFTISRDNAKNTVYLQMSSLRAEDTAVYYCNYINT |
| | | DNEIGGPWGQGTLVTVRP |
| 37 | Siglec8-Tet- | QVQLLESGGGEVQPGGSLRLSCAASGSISIFSDYVVAWYRQAPGKEREWVA |
| | hzG2v55 VHH | FIQRGGTANYAESAKGRFTISRDNAKNTVYLQMSSLRAEDTAVYYCNYINT |
| | | DNEIGGPWGQGTLVTVRPGGSGGSQVQLLESGGGEVQPGGSLRLSCAASGS |
| | | ISIFSDYVVAWYRQAPGKEREWVAFIQRGGTANYAESAKGRFTISRDNAKN |
| | | TVYLQMSSLRAEDTAVYYCNYINTDNEIGGPWGQGTLVTVRP |
| 38 | hzG2v55 CDR1 | GSISIFSDYVVA |
| 39 | hzG2v55 CDR2 | FIQRGGTAN |
| 40 | hzG2v55 CDR3 | INTDNEIGGP |
| 46 | hzG2 CDR1 | GSISIFSXYVVA, wherein X is M, K, D, Y, L, or I |
| | consensus | |
| | (AbM) | |
| 47 | hzG2 CDR2 | FIQXGGTAN, wherein X is R or S |
| | consensus | |
| | (AbM) | |
| 48 | hzG2 CDR3 | IX₁X₂DNEIGGP, wherein X₁ is N, Q, S, or T; and X₂ is S or T |
| | consensus | |
| | (AbM) | |
| 49 | Siglec8-B12 | QVQVVESGGGLVQPGGSLRLSCAASGSTSLLNAMAWYRQAPGKERELVAAI |
| | VHH | SSGGGTYYADSVKGRFTISTNNAKNTLYLQMNSLKPEDTAMYYCAPGPNYS |
| | | DYEEINWGQGTQVTVKP |
| 50 | Siglec8-Tet-B12 | QVQVVESGGGLVQPGGSLRLSCAASGSTSLLNAMAWYRQAPGKERELVAAI |
| | VHH | SSGGGTYYADSVKGRFTISTNNAKNTLYLQMNSLKPEDTAMYYCAPGPNYS |
| | | DYEEINWGQGTQVTVKPGGSGGSQVQVVESGGGLVQPGGSLRLSCAASGST |
| | | SLLNAMAWYRQAPGKERELVAAISSGGGTYYADSVKGRFTISTNNAKNTLY |
| | | LQMNSLKPEDTAMYYCAPGPNYSDYEEINWGQGTQVTVKP |
| 51 | Siglec8-Hex- | QVQVVESGGGLVQPGGSLRLSCAASGSTSLLNAMAWYRQAPGKERELVAAI |
| | B12 VHH | SSGGGTYYADSVKGRFTISTNNAKNTLYLQMNSLKPEDTAMYYCAPGPNYS |
| | | DYEEINWGQGTQVTVKPGGSGGSQVQVVESGGGLVQPGGSLRLSCAASGST |
| | | SLLNAMAWYRQAPGKERELVAAISSGGGTYYADSVKGRFTISTNNAKNTLY |
| | | LQMNSLKPEDTAMYYCAPGPNYSDYEEINWGQGTQVTVKPGGSGGSQVQVV |
| | | ESGGGLVQPGGSLRLSCAASGSTSLLNAMAWYRQAPGKERELVAAISSGGG |
| | | TYYADSVKGRFTISTNNAKNTLYLQMNSLKPEDTAMYYCAPGPNYSDYEEI |
| | | NWGQGTQVTVKP |
| 52 | Siglec8-B12 | GSTSLLNAMA |
| | CDR1 | |
| 53 | Siglec8-B12 | AISSGGGTY |
| | CDR2 | |
| 54 | Siglec8-B12 | GPNYSDYEEIN |
| | CDR3 | |
| 55 | Siglec8-D1 VHH | QVQLVESGGGLVQPGGSLRLSCAASGSIASIHGMGWYRQAPGKERELVAAI |
| | | AGEGSTYYADSVKGRFTISRDFAKNTLYLQMNNLKAEDTAMYYCANNDERD |
| | | DYWGQGTQVTVKP |
| 56 | Siglec8-Tet-D1 | QVQLVESGGGLVQPGGSLRLSCAASGSIASIHGMGWYRQAPGKERELVAAI |
| | VHH | AGEGSTYYADSVKGRFTISRDFAKNTLYLQMNNLKAEDTAMYYCANNDERD |
| | | DYWGQGTQVTVKPGGSGGSQVQLVESGGGLVQPGGSLRLSCAASGSIASIH |
| | | GMGWYRQAPGKERELVAAIAGEGSTYYADSVKGRFTISRDFAKNTLYLQMN |
| | | NLKAEDTAMYYCANNDERDDYWGQGTQVTVKP |
| 57 | Siglec8-Hex-D1 | QVQLVESGGGLVQPGGSLRLSCAASGSIASIHGMGWYRQAPGKERELVAAI |
| | VHH | AGEGSTYYADSVKGRFTISRDFAKNTLYLQMNNLKAEDTAMYYCANNDERD |
| | | DYWGQGTQVTVKPGGSGGSQVQLVESGGGLVQPGGSLRLSCAASGSIASIH |
| | | GMGWYRQAPGKERELVAAIAGEGSTYYADSVKGRFTISRDFAKNTLYLQMN |
| | | NLKAEDTAMYYCANNDERDDYWGQGTQVTVKPGGSGGSQVQLVESGGGLVQ |
| | | PGGSLRLSCAASGSIASIHGMGWYRQAPGKERELVAAIAGEGSTYYADSVK |
| | | GRFTISRDFAKNTLYLQMNNLKAEDTAMYYCANNDERDDYWGQGTQVTVKP |
| 58 | Siglec8-D1 | GSIASIHGMG |
| | CDR1 | |
| 59 | Siglec8-D1 | AIAGEGSTY |
| | CDR2 | |
| 60 | Siglec8-D1 | NDERDDY |
| | CDR3 | |
| 61 | Siglec8-E2 VHH | QVQLVESGGGLVQPGGSLRLSCATSGIIFTSNVINWYRQAPGKQREFVAFI |
| | | TSITSGQSTMYADAVKGRFTVSRDNDKNIGYLQMTSLQPEDTARYVCNVGA |
| | | YWGQGTQVTVKP |
| 62 | Siglec8-Tet-E2 | QVQLVESGGGLVQPGGSLRLSCATSGIIFTSNVINWYRQAPGKQREFVAFI |
| | VHH | TSITSGQSTMYADAVKGRFTVSRDNDKNIGYLQMTSLQPEDTARYVCNVGA |
| | | YWGQGTQVTVKPGGSGGSQVQLVESGGGLVQPGGSLRLSCATSGIIFTSNV |
| | | INWYRQAPGKQREFVAFITSITSGQSTMYADAVKGRFTVSRDNDKNIGYLQ |
| | | MTSLQPEDTARYVCNVGAYWGQGTQVTVKP |
| 165 | Siglec8-Hex-E2 | QVQLVESGGGLVQPGGSLRLSCATSGIIFTSNVINWYRQAPGKQREFVAFI |
| | VHH | TSITSGQSTMYADAVKGRFTVSRDNDKNIGYLQMTSLQPEDTARYVCNVGA |
| | | YWGQGTQVTVKPGGSGGSQVQLVESGGGLVQPGGSLRLSCATSGIIFTSNV |
| | | INWYRQAPGKQREFVAFITSITSGQSTMYADAVKGRFTVSRDNDKNIGYLQ |
| | | MTSLQPEDTARYVCNVGAYWGQGTQVTVKPGGSGGSQVQLVESGGGLVQPG |
| | | GSLRLSCATSGIIFTSNVINWYRQAPGKQREFVAFITSITSGQSTMYADAV |
| | | KGRFTVSRDNDKNIGYLQMTSLQPEDTARYVCNVGAYWGQGTQVTVKP |
| 63 | Siglec8-E2 | GIIFTSNVIN |
| | CDR1 | |
| 64 | Siglec8-E2 | FITSITSGQSTM |
| | CDR2 | |
| n/a | Siglec8-E2 | GAY |
| | CDR3 | |
| 41 | Siglec8-ECD- | MEGDRQYGDGYLLQVQELVTVQEGLCVHVPCSFSYPQDGWTDSDPVHGYWF |
| | LFc | RAGDRPYQDAPVATNNPDREVQAETQGRFQLLGDIWSNDCSLSIRDARKRD |
| | | KGSYFFRLERGSMKWSYKSQLNYKTKQLSVFVTALTHRPDILILGTLESGH |
| | | SRNLTCSVPWACKQGTPPMISWIGASVSSPGPTTARSSVLTLTPKPQDHGT |
| | | SLTCQVTLPGTGVTTTSTVRLDVSYPPWNLTMTVFQGDATASTALGNGSSL |
| | | SVLEGQSLRLVCAVNSNPPARLSWTRGSLTLCPSRSSNPGLLELPRVHVRD |
| | | EGEFTCRAQNAQGSQHISLSLSLQNEGTGTSRPVSQVTLAAGTGGSGGGGC |
| | | PPCPAPELPGGPSVFVFPPKPKDVLSISGRPEVTCVVVDVGKEDPEVNENW |
| | | YIDGVEVRTANTKPKEEQFNSTYRVVSVLPIQHQDWLTGKEFKCKVNNKAL |
| | | PAPIERTISKAKGQTREPQVYTLAPHREELAKDTVSVTCLVKGFYPADINV |
| | | EWQRNGQPESEGTYANTPPQLDNDGTYFLYSKLSVGKNTWQRGETLTCVVM |
| | | HEALHNHYTQKSISQSLGK |
| 42 | Human Siglec8 | MLLLLLLLPLLWGTKGMEGDRQYGDGYLLQVQELVTVQEGLCVHVPCSFSY |
| | | PQDGWTDSDPVHGYWFRAGDRPYQDAPVATNNPDREVQAETQGRFQLLGDI |
| | | WSNDCSLSIRDARKRDKGSYFFRLERGSMKWSYKSQLNYKTKQLSVFVTAL |
| | | THRPDILILGTLESGHSRNLTCSVPWACKQGTPPMISWIGASVSSPGPTTA |
| | | RSSVLTLTPKPQDHGTSLTCQVTLPGTGVTTTSTVRLDVSYPPWNLTMTVF |
| | | QGDATASTALGNGSSLSVLEGQSLRLVCAVNSNPPARLSWTRGSLTLCPSR |
| | | SSNPGLLELPRVHVRDEGEFTCRAQNAQGSQHISLSLSLQNEGTGTSRPVS |
| | | QVTLAAVGGAGATALAFLSFCIIFIIVRSCRKKSARPAAGVGDTGMEDAKA |
| | | IRGSASQGPLTESWKDGNPLKKPPPAVAPSSGEEGELHYATLSFHKVKPQD |
| | | PQGQEATDSEYSEIKIHKRETAETQACLRNHNPSSKEVRG |
| 43 | Mature human | MEGDRQYGDGYLLQVQELVTVQEGLCVHVPCSFSYPQDGWTDSDPVHGYWF |
| | Siglec8 (amino | RAGDRPYQDAPVATNNPDREVQAETQGRFQLLGDIWSNDCSLSIRDARKRD |
| | acids 17-499) | KGSYFFRLERGSMKWSYKSQLNYKTKQLSVFVTALTHRPDILILGTLESGH |
| | | SRNLTCSVPWACKQGTPPMISWIGASVSSPGPTTARSSVLTLTPKPQDHGT |
| | | SLTCQVTLPGTGVTTTSTVRLDVSYPPWNLTMTVFQGDATASTALGNGSSL |
| | | SVLEGQSLRLVCAVNSNPPARLSWTRGSLTLCPSRSSNPGLLELPRVHVRD |
| | | EGEFTCRAQNAQGSQHISLSLSLQNEGTGTSRPVSQVTLAAVGGAGATALA |
| | | FLSFCIIFIIVRSCRKKSARPAAGVGDTGMEDAKAIRGSASQGPLTESWKD |
| | | GNPLKKPPPAVAPSSGEEGELHYATLSFHKVKPQDPQGQEATDSEYSEIKI |
| | | HKRETAETQACLRNHNPSSKEVRG |
| 44 | human IgG1 Fc | DKTHTCPPCPAPELLGGPSVFLFPPKPKDTLMISRTPEVTCVVVDVSHEDP |
| | region | EVKFNWYVDGVEVHNAKTKPREEQYNSTYRVVSVLTVLHQDWLNGKEYKCK |
| | | VSNKALPAPIEKTISKAKGQPREPQVYTLPPSRDELTKNQVSLTCLVKGFY |
| | | PSDIAVEWESNGQPENNYKTTPPVLDSDGSFFLYSKLTVDKSRWQQGNVFS |
| | | CSVMHEALHNHYTQKSLSLSPGK |
| 45 | human IgG1 Fc | DKTHTCPPCPAPGGPSVFLFPPKPKDTLMISRTPEVTCVVVDVSHEDPEVK |
| | xELL | FNWYVDGVEVHNAKTKPREEQYNSTYRVVSVLTVLHQDWLNGKEYKCKVSN |
| | | KALPAPIEKTISKAKGQPREPQVYTLPPSRDELTKNQVSLTCLVKGFYPSD |
| | | IAVEWESNGQPENNYKTTPPVLDSDGSFFLYSKLTVDKSRWQQGNVFSCSV |
| | | MHEALHNHYTQKSLSLSPGK |
| 65 | Human IgG1 Fc | DKTHTCPPCPAPELLGGPDVFLFPPKPKDTLMISRTPEVTCVVVDVSHEDP |
| | S239D, I332E | EVKFNWYVDGVEVHNAKTKPREEQYNSTYRVVSVLTVLHQDWLNGKEYKCK |
| | (SDIE) | VSNKALPAPEEKTISKAKGQPREPQVYTLPPSRDELTKNQVSLTCLVKGFY |
| | | PSDIAVEWESNGQPENNYKTTPPVLDSDGSFFLYSKLTVDKSRWQQGNVFS |
| | | CSVMHEALHNHYTQKSLSLSPGK |
| 66 | human IgG1 Fc | DKTHTCPPCPAPELLGGPSVFLFPPKPKDTLMISRTPEVTCVVVDVSHEDP |
| | region-K447 | EVKFNWYVDGVEVHNAKTKPREEQYNSTYRVVSVLTVLHQDWLNGKEYKCK |
| | | VSNKALPAPIEKTISKAKGQPREPQVYTLPPSRDELTKNQVSLTCLVKGFY |
| | | PSDIAVEWESNGQPENNYKTTPPVLDSDGSFFLYSKLTVDKSRWQQGNVFS |
| | | CSVMHEALHNHYTQKSLSLSPG |
| 67 | human IgG1 Fc | DKTHTCPPCPAPGGPSVFLFPPKPKDTLMISRTPEVTCVVVDVSHEDPEVK |
| | xELL-K447 | FNWYVDGVEVHNAKTKPREEQYNSTYRVVSVLTVLHQDWLNGKEYKCKVSN |
| | | KALPAPIEKTISKAKGQPREPQVYTLPPSRDELTKNQVSLTCLVKGFYPSD |
| | | IAVEWESNGQPENNYKTTPPVLDSDGSFFLYSKLTVDKSRWQQGNVFSCSV |
| | | MHEALHNHYTQKSLSLSPG |
| 68 | Human IgG1 Fc | DKTHTCPPCPAPELLGGPDVFLFPPKPKDTLMISRTPEVTCVVVDVSHEDP |
| | S239D, I332E | EVKFNWYVDGVEVHNAKTKPREEQYNSTYRVVSVLTVLHQDWLNGKEYKCK |
| | (SDIE)-K447 | VSNKALPAPEEKTISKAKGQPREPQVYTLPPSRDELTKNQVSLTCLVKGFY |
| | | PSDIAVEWESNGQPENNYKTTPPVLDSDGSFFLYSKLTVDKSRWQQGNVFS |
| | | CSVMHEALHNHYTQKSLSLSPG |
| 69 | Linker 1 | GGSGGS |
| 70 | Linker 2 | GGGG |
| 71 | cx11914 | EVQLLESGGGEVQPGGSLRLSCAASGSISIFSKYVVAWYRQAPGKEREWVA |
| | | FIQSGGTANYAESAKGRFTISRDNAKNTVYLQMSSLRAEDTAVYYCNYINT |
| | | DNEIGGPWGQGTLVTVRPGGSGGSEVQLLESGGGEVQPGGSLRLSCAASGS |
| | | ISIFSKYVVAWYRQAPGKEREWVAFIQSGGTANYAESAKGRFTISRDNAKN |
| | | TVYLQMSSLRAEDTAVYYCNYINTDNEIGGPWGQGTLVTVRPGGGGDKTHT |
| | | CPPCPAPELLGGPDVFLFPPKPKDTLMISRTPEVTCVVVDVSHEDPEVKEN |
| | | WYVDGVEVHNAKTKPREEQYNSTYRVVSVLTVLHQDWLNGKEYKCKVSNKA |
| | | LPAPEEKTISKAKGQPREPQVYTLPPSRDELTKNQVSLTCLVKGFYPSDIA |
| | | VEWESNGQPENNYKTTPPVLDSDGSFFLYSKLTVDKSRWQQGNVFSCSVMH |
| | | EALHNHYTQKSLSLSPGK |
| 72 | cx12532 | EVQLLESGGGEVQPGGSLRLSCAASGSISIFSKYVVAWYRQAPGKEREWVA |
| | | FIQSGGTANYAESAKGRFTISRDNAKNTVYLQMSSLRAEDTAVYYCNYINT |
| | | DNEIGGPWGQGTLVTVRPGGSGGSEVQLLESGGGEVQPGGSLRLSCAASGS |
| | | ISIFSKYVVAWYRQAPGKEREWVAFIQSGGTANYAESAKGRFTISRDNAKN |
| | | TVYLQMSSLRAEDTAVYYCNYINTDNEIGGPWGQGTLVTVRPGGGGDKTHT |
| | | CPPCPAPELLGGPSVFLFPPKPKDTLMISRTPEVTCVVVDVSHEDPEVKEN |
| | | WYVDGVEVHNAKTKPREEQYNSTYRVVSVLTVLHQDWLNGKEYKCKVSNKA |
| | | LPAPIEKTISKAKGQPREPQVYTLPPSRDELTKNQVSLTCLVKGFYPSDIA |
| | | VEWESNGQPENNYKTTPPVLDSDGSFFLYSKLTVDKSRWQQGNVFSCSVMH |
| | | EALHNHYTQKSLSLSPGK |
| 73 | cx12562 | EVQLLESGGGEVQPGGSLRLSCAASGSISIFSKYVVAWYRQAPGKEREWVA |
| | | FIQSGGTANYAESAKGRFTISRDNAKNTVYLQMSSLRAEDTAVYYCNYINT |
| | | DNEIGGPWGQGTLVTVRPGGGGDKTHTCPPCPAPELLGGPDVFLFPPKPKD |
| | | TLMISRTPEVTCVVVDVSHEDPEVKFNWYVDGVEVHNAKTKPREEQYNSTY |
| | | RVVSVLTVLHQDWLNGKEYKCKVSNKALPAPEEKTISKAKGQPREPQVYTL |
| | | PPSRDELTKNQVSLTCLVKGFYPSDIAVEWESNGQPENNYKTTPPVLDSDG |
| | | SFFLYSKLTVDKSRWQQGNVFSCSVMHEALHNHYTQKSLSLSPGK |
| 74 | Siglec8-G2 | GSISIFSMY |
| | CDR1 (Chothia) | |
| 75 | hzG2v47 CDR1 | GSISIFSDY |
| | (Chothia) | |
| 76 | hzG2v51 CDR1 | GSISIFSKY |
| | (Chothia) | |
| 77 | Siglec8-B12 | GSTSLLN |
| | CDR1 (Chothia) | |
| 78 | Siglec8-D1 | GSIASIH |
| | CDR1 (Chothia) | |
| 79 | Siglec8-E2 | GIIFTSN |
| | CDR1 (Chothia) | |
| 80 | Siglec8-G2 | QRGGT |
| | CDR2 (Chothia) | |
| 81 | hzG2v53 CDR2 | QSGGT |
| | (Chothia) | |
| 82 | Siglec8-B12 | SSGGG |
| | CDR2 (Chothia) | |
| 83 | Siglec8-D1 | AGEGS |
| | CDR2 (Chothia) | |
| 84 | Siglec8-E2 | TSITSGQS |
| | CDR2 (Chothia) | |
| 85 | Siglec8-G2 | FSMYVVA |
| | CDR1 (Kabat) | |
| 86 | hzG2v47 CDR1 | FSDYVVA |
| | (Kabat) | |
| 87 | hzG2v52 CDR1 | FSKYVVA |
| | (Kabat) | |
| 88 | Siglec8-B12 | LNAMA |
| | CDR1 (Kabat) | |
| 89 | Siglec8-D1 | IHGMG |
| | CDR1 (Kabat) | |
| 90 | Siglec8-E2 | SNVIN |
| | CDR1 (Kabat) | |
| 91 | Siglec8-G2 | FIQRGGTANYADSAKG |
| | CDR2 (Kabat) | |
| 92 | hzG2v14 CDR2 | FIQRGGTANYAESAKG |
| | (Kabat) | |
| 93 | hzG2v51 CDR2 | FIQRGGTANYAESAKD |
| | (Kabat) | |
| 94 | hzG2v53 CDR2 | FIQSGGTANYAESAKG |
| | (Kabat) | |
| 95 | Siglec8-B12 | AISSGGGTYYADSVKG |
| | CDR2 (Kabat) | |
| 96 | Siglec8-D1 | AIAGEGSTYYADSVKG |
| | CDR2 (Kabat) | |
| 97 | Siglec8-E2 | FITSITSGQSTMYADAVKG |
| | CDR2 (Kabat) | |
| 98 | Siglec8-G2 | IFSMYVVA |
| | CDR1 (Contact) | |
| 99 | hzG2v47 CDR1 | IFSDYVVA |
| | (Contact) | |
| 100 | hzG2v51 CDR1 | IFSKYVVA |
| | (Contact) | |
| 101 | Siglec8-B12 | LLNAMA |
| | CDR1 (Contact) | |
| 102 | Siglec8-D1 | SIHGMG |
| | CDR1 (Contact) | |
| 103 | Siglec8-E2 | TSNVIN |
| | CDR1 (Contact) | |
| 104 | Siglec8-G2 | WVAFIQRGGTAN |
| | CDR2 (Contact) | |
| 105 | hzG2v53 CDR2 | WVAFIQSGGTAN |
| | (Contact) | |
| 106 | Siglec8-B12 | LVAAISSGGGTY |
| | CDR2 (Contact) | |
| 107 | Siglec8-D1 | LVAAIAGEGSTY |
| | CDR2 (Contact) | |
| 108 | Siglec8-E2 | FVAFITSITSGQSTM |
| | CDR2 (Contact) | |
| 109 | Siglec8-G2 | NYINSDNEIGG |
| | CDR3 (Contact) | |
| 110 | hzG2v14 CDR3 | NYINTDNEIGG |
| | (Contact) | |
| 111 | Siglec8-B12 | APGPNYSDYEEI |
| | CDR3 (Contact) | |
| 112 | Siglec8-D1 | ANNDERDD |
| | CDR3 (Contact) | |
| 113 | Siglec8-E2 | NVGA |
| | CDR3 (Contact) | |
| 114 | Siglec8-G2 | GSISIFSMYV |
| | CDR1 (IMGT) | |
| 115 | hzG2v47 CDR1 | GSISIFSDYV |
| | (IMGT) | |
| 116 | hzG2v51 CDR1 | GSISIFSKYV |
| | (IMGT) | |
| 117 | Siglec8-B12 | GSTSLLNA |
| | CDR1 (IMGT) | |
| 118 | Siglec8-D1 | GSIASIHG |
| | CDR1 (IMGT) | |
| 119 | Siglec8-E2 | GIIFTSNV |
| | CDR1 (IMGT) | |
| 120 | Siglec8-G2 | IQRGGTA |
| | CDR2 (IMGT) | |
| 121 | hzG2v53 CDR2 | IQSGGTA |
| | (IMGT) | |
| 122 | Siglec8-B12 | ISSGGGT |
| | CDR2 (IMGT) | |
| 123 | Siglec8-D1 | IAGEGST |
| | CDR2 (IMGT) | |
| 124 | Siglec8-E2 | ITSITSGQST |
| | CDR2 (IMGT) | |
| 125 | Siglec8-G2 | NYINSDNEIGGP |
| | CDR3 (IMGT) | |
| 126 | hzG2v14 CDR3 | NYINTDNEIGGP |
| | (IMGT) | |
| 127 | Siglec8-B12 | APGPNYSDYEEIN |
| | CDR3 (IMGT) | |
| 128 | Siglec8-D1 | ANNDERDDY |
| | CDR3 (IMGT) | |
| 129 | Siglec8-E2 | NVGAY |
| | CDR3 (IMGT) | |
| 130 | Siglec8 hzG2v11 | EVQLLESGGGEVQPGGSLRLSCAASGSISIFSMYVVAWYRQAPGKEREWVA |
| | | FIQRGGTANYAESAKGRFTISRDNAKNTVYLQMSSLRAEDTAVYYCNYIQS |
| | | DNEIGGPWGQGTLVTVKP |
| 131 | Siglec8 hzG2v12 | EVQLLESGGGEVQPGGSLRLSCAASGSISIFSMYVVAWYRQAPGKEREWVA |
| | | FIQRGGTANYAESAKGRFTISRDNAKNTVYLQMSSLRAEDTAVYYCNYISS |
| | | DNEIGGPWGQGTLVTVKP |
| 132 | Siglec8 hzG2v13 | EVQLLESGGGEVQPGGSLRLSCAASGSISIFSMYVVAWYRQAPGKEREWVA |
| | | FIQRGGTANYAESAKGRFTISRDNAKNTVYLQMSSLRAEDTAVYYCNYITS |
| | | DNEIGGPWGQGTLVTVKP |
| 133 | Siglec8 hzG2v15 | EVQLLESGGGEVQPGGSLRLSCAASGSISIFSYYVVAWYRQAPGKEREWVA |
| | | FIQRGGTANYAESAKGRFTISRDNAKNTVYLQMSSLRAEDTAVYYCNYINS |
| | | DNEIGGPWGQGTLVTVKP |
| 134 | Siglec8 hzG2v16 | EVQLLESGGGEVQPGGSLRLSCAASGSISIFSLYVVAWYRQAPGKEREWVA |
| | | FIQRGGTANYAESAKGRFTISRDNAKNTVYLQMSSLRAEDTAVYYCNYINS |
| | | DNEIGGPWGQGTLVTVKP |
| 135 | Siglec8 hzG2v17 | EVQLLESGGGEVQPGGSLRLSCAASGSISIFSIYVVAWYRQAPGKEREWVA |
| | | FIQRGGTANYAESAKGRFTISRDNAKNTVYLQMSSLRAEDTAVYYCNYINS |
| | | DNEIGGPWGQGTLVTVKP |
| 136 | Siglec8 hzG2v18 | EVQLLESGGGEVQPGGSLRLSCAASGSISIFSKYVVAWYRQAPGKEREWVA |
| | | FIQRGGTANYAESAKGRFTISRDNAKNTVYLQMSSLRAEDTAVYYCNYINS |
| | | DNEIGGPWGQGTLVTVKP |
| 137 | Siglec8 hzG2v19 | EVQLLESGGGEVQPGGSLRLSCAASGSISIFSSYAVAWYRQAPGKEREWVA |
| | | FIQRGGTANYAESAKGRFTISRDNAKNTVYLQMSSLRAEDTAVYYCNYINS |
| | | DNEIGGPWGQGTLVTVKP |
| 138 | Siglec8 hzG2v20 | EVQLLESGGGEVQPGGSLRLSCAASGSISIFSSYAMAWYRQAPGKEREWVA |
| | | FIQRGGTANYAESAKGRFTISRDNAKNTVYLQMSSLRAEDTAVYYCNYINS |
| | | DNEIGGPWGQGTLVTVKP |
| 139 | Siglec8 hzG2v21 | EVQLLESGGGEVQPGGSLRLSCAASGSISIFSSYAMSWYRQAPGKEREWVA |
| | | FIQRGGTANYAESAKGRFTISRDNAKNTVYLQMSSLRAEDTAVYYCNYINS |
| | | DNEIGGPWGQGTLVTVKP |
| 140 | Siglec8 hzG2v24 | QVTLKESGGGLVRTGGSLRLSCAASGSISIFSMYVVAWYRQAPGKQREWVA |
| | | FIQRGGTANYAESAKGRFTISRDNAKNTVYLQMSSLRAEDTAVYYCNYINS |
| | | DNEIGGPWGQGTLVTVKP |
| 141 | Siglec8 hzG2v25 | EVQLLESGGGLVQTGGSLRLSCAASGSISIFSMYVVAWYRQAPGKQREWVA |
| | | FIQRGGTANYAESAKGRFTISRDNAKNTVYLQMSSLRAEDTAVYYCNYINS |
| | | DNEIGGPWGQGTLVTVKP |
| 142 | Siglec8 hzG2v26 | EVQLLESGGGLVRPGGSLRLSCAASGSISIFSMYVVAWYRQAPGKQREWVA |
| | | FIQRGGTANYAESAKGRFTISRDNAKNTVYLQMSSLRAEDTAVYYCNYINS |
| | | DNEIGGPWGQGTLVTVKP |
| 143 | Siglec8 hzG2v28 | EVQLLESGGGEVQTGGSLRLSCAASGSISIFSMYVVAWYRQAPGKQREWVA |
| | | FIQRGGTANYAESAKGRFTISRDNAKNTVYLQMSSLRAEDTAVYYCNYINS |
| | | DNEIGGPWGQGTLVTVKP |
| 144 | Siglec8 hzG2v29 | EVQLLESGGGEVRPGGSLRLSCAASGSISIFSMYVVAWYRQAPGKQREWVA |
| | | FIQRGGTANYAESAKGRFTISRDNAKNTVYLQMSSLRAEDTAVYYCNYINS |
| | | DNEIGGPWGQGTLVTVKP |
| 145 | Siglec8 hzG2v30 | EVQLLESGGGLVRIGGSLRLSCAASGSISIFSMYVVAWYRQAPGKQREWVA |
| | | FIQRGGTANYAESAKGRFTISRDNAKNTVYLQMSSLRAEDTAVYYCNYINS |
| | | DNEIGGPWGQGTLVTVKP |
| 146 | Siglec8 hzG2v31 | EVQLLESGGGLVRIGGSLRLSCAASGSISIFSMYVVAWYRQAPGKQREWVA |
| | | FIQRGGTANYAESAKGRFTISRDNAKNTVYLQMSSLRAEDTAVYYCNYINS |
| | | DNEIGGPWGQGTLVTVKP |
| 147 | Siglec8 hzG2v32 | EVQLLESGGGEVQTGGSLRLSCAASGSISIFSMYVVAWYRQAPGKQREWVA |
| | | FIQRGGTANYAESAKGRFTISRDNAKNTVYLQMSSLRPEDTAVYYCNYINS |
| | | DNEIGGPWGQGTLVTVKP |
| 148 | Siglec8 hzG2v33 | EVQLLESGGGEVQTGGSLRLSCAASGSISIFSMYVVAWYRQAPGKQREWVA |
| | | FIQRGGTANYAESAKGRFTISRDNAKNTVYLQMSSLRADDTAVYYCNYINS |
| | | DNEIGGPWGQGTLVTVKP |
| 149 | hzG2 CDR1 | GSISIFSXY, wherein X is M, K, D, Y, L, or I |
| | consensus | |
| | (Chothia) | |
| 150 | hzG2 CDR2 | QXGGT, wherein X is R or S |
| | consensus | |
| | (Chothia) | |
| 151 | hzG2 CDR3 | IX₁X₂DNEIGGP, wherein X₁ is N, Q, S, or T; and X₂ is S or T |
| | consensus | |
| | (Chothia) | |
| 152 | hzG2 CDR1 | FSXYVVA, wherein X is M, K, D, Y, L, or I |
| | consensus | |
| | (Kabat) | |
| 153 | hzG2 CDR2 | FIQX₁GGTANYAESAKX₂, wherein X₁ is R or S; and X₂ is G or D |
| | consensus | |
| | (Kabat) | |
| 154 | hzG2 CDR3 | IX₁X₂DNEIGGP, wherein X₁ is N, Q, S, or T; and X₂ is S or T |
| | consensus | |
| | (Kabat) | |
| 155 | hzG2 CDR1 | IFSXYVVA, wherein X is M, K, D, Y, L, or I |
| | consensus | |
| | (Contact) | |
| 156 | hzG2 CDR2 | WVX₁FIQX₂GGTAN, wherein X₁ is A or S; and X₂ is R or S |
| | consensus | |
| | (Contact) | |
| 157 | hzG2 CDR3 | NYIX₁X₂DNEIGG, wherein X₁ is N, Q, S, or T; and X₂ is S or T |
| | consensus | |
| | (Contact) | |
| 158 | hzG2 CDR1 | GSISIFSXYV, wherein X is M, K, D, Y, L, or I |
| | consensus | |
| | (IMGT) | |
| 159 | hzG2 CDR2 | IQXGGTA, wherein X is R or S |
| | consensus | |
| | (IMGT) | |
| 160 | hzG2 CDR3 | NYIX₁X₂DNEIGGP, wherein X₁ is N, Q, S, or T; and X₂ is S or T |
| | consensus | |
| | (IMGT) | |
| 161 | Siglec8 | EVQLLESGGGEVQPGGSLRLSCAASGSISIFSMYVVAWYRQAPGKEREWVA |
| | hzG2v2.1 | FIQRGGTANYAESAKGRFTISRDNAKNTVYLQMSSLRAEDTAVYYCNYINS |
| | | DNEIGGPWGQGTLVTVKP |
| 162 | Siglec8 | EVQLLESGGGEVQPGGSLRLSCAASGSISIFSMYVVAWYRQAPGKQREWVS |
| | hzG2v5.1 | AIQRGGTANYAESAKGRFTISRDNAKNTVYLQMSSLRAEDTAVYYCNYINS |
| | | DNEIGGPWGQGTLVTVKP |
| 163 | Siglec8 | EVQLLESGGGEVQPGGSLRLSCAASGSISIFSMYVVAWYRQAPGKQREWVS |
| | hzG2v6.1 | FIQRGGTANYAESAKGRFTISRDNAKNTVYLQMSSLRAEDTAVYYCNYINS |
| | | DNEIGGPWGQGTLVTVKP |
| 164 | Siglec8 | QVTLKESGGGEVQPGGSLRLSCAASGSISIFSMYVVAWYRQAPGKQREWVA |
| | hzG2v22 | FIQRGGTANYAESAKGRFTISRDNAKNTVYLQMSSLRAEDTAVYYCNYINS |
| | | DNEIGGPWGQGTLVTVKP |

1. An VHH domain that binds Siglec-8, comprising complementarity determining region 1 (CDR1), complementarity determining region 2 (CDR2), and complementarity determining region 3 (CDR3) sequences of a VHH that comprises an amino acid sequence selected from the group consisting of SEQ ID NO: 26, 5, 11, 16, 21, 31, 36, 130-138, 140-148, 161, 163, and 164.

2. An VHH domain that binds Siglec-8, comprising CDR1, CDR2, and CDR3 sequences set forth in:
a) SEQ ID NOs: 46, 47, and 48, respectively, as defined according to the AbM numbering system;
b) SEQ ID NOs: 149, 150, and 151, respectively, as defined according to the Chothia numbering system;
c) SEQ ID NOs: 152, 153, and 154, respectively, as defined according to the Kabat numbering system;
d) SEQ ID NOs: 155, 156, and 157, respectively, as defined according to the Contact numbering system; or
e) SEQ ID NOs: 158, 159, and 160, respectively, as defined according to the IMGT numbering system.

3. The VHH domain of claim 2, wherein:
a) the CDR1 comprises the amino acid sequence of SEQ ID NO: 18, 2, or 13; the CDR2 comprises the amino acid sequence of SEQ ID NO: 29 or 3; and the CDR3 comprises the amino acid sequence of SEQ ID NO: 8, as defined according to the AbM numbering system;
b) the CDR1 comprises the amino acid sequence of SEQ ID NO: 74, 75, or 76; the CDR2 comprises the amino acid sequence of SEQ ID NO: 80 or 81; and the CDR3 comprises the amino acid sequence of SEQ ID NO: 8, as defined according to the Chothia numbering system;
c) the CDR1 comprises the amino acid sequence of SEQ ID NO: 85, 86, or 87; the CDR2 comprises the amino acid sequence of SEQ ID NO: 92, 93, or 94; and the CDR3 comprises the amino acid sequence of SEQ ID NO: 8, as defined according to the Kabat numbering system;
d) the CDR1 comprises the amino acid sequence of SEQ ID NO: 98, 99, or 100; the CDR2 comprises the amino acid sequence of SEQ ID NO: 104 or 105; and the CDR3 comprises the amino acid sequence of SEQ ID NO: 110, as defined according to the Contact numbering system; or
e) the CDR1 comprises the amino acid sequence of SEQ ID NO: 114, 115, or 116;
the CDR2 comprises the amino acid sequence of SEQ ID NO: 120 or 121; and the CDR3 comprises the amino acid sequence of SEQ ID NO: 126, as defined according to the IMGT numbering system.

4. The VHH domain of claim 2, wherein the CDR1, CDR2, and CDR3 sequences are set forth in:
a) SEQ ID NOs: 18, 29, and 8; 2, 3, and 8; 13, 3, and 8; or 18, 3, and 8, respectively, as defined according to the AbM numbering system;
b) SEQ ID NOs: 74, 80, and 8; 75, 80, and 8; 76, 80, and 8; or 76, 81, and 8, respectively, as defined according to the Chothia numbering system;
c) SEQ ID NOs: 85, 92, and 8; 86, 92, and 8; 87, 93, and 8; 87, 92, and 8; or 87, 94, and 8, respectively, as defined according to the Kabat numbering system;
d) SEQ ID NOs: 98, 104, and 110; 99, 104, and 110; 100, 104, and 110; or 100, 105, and 110, respectively, as defined according to the Contact numbering system; or
e) SEQ ID NOs: 114, 120, and 126; 115, 120, and 126; 116, 120, and 126; or 116, 121, and 126, respectively, as defined according to the IMGT numbering system.

5-8. (canceled)

9. The VHH domain of claim 2, comprising an amino acid sequence that is at least 80%, at least 85%, at least 90%, at least 95%, at least 96%, at least 97%, at least 98%, at least 99%, or 100% identical to an amino acid sequence selected from the group consisting of SEQ ID NO: 26, 5, 11, 16, 21, 31, 36, 130-138, 140-148, 161, 163, and 164.

10. The VHH domain of claim 2, wherein the VHH domain is humanized.

11. An VHH domain that binds Siglec-8, comprising CDR1, CDR2, and CDR3 sequences of a VHH that comprises the amino acid sequence of SEQ ID NO: 1, 49 or 61.

12-22. (canceled)

23. A polypeptide comprising the VHH domain of claim 2.

24. The polypeptide of claim 23, comprising at least two VHH domains of claim 2.

25. The polypeptide of claim 24, wherein the two VHH domains are operably linked to each other via a peptide linker, a peptide linker comprising the amino acid sequence of SEQ ID NO: 69, or a peptide linker consisting of the amino acid sequence of SEQ ID NO: 69.

26-27. (canceled)

28. The polypeptide of claim 24, wherein the VHH domains comprise the same VHH amino acid sequence.

29. The polypeptide of claim 24, wherein the polypeptide comprises an amino acid sequence that is at least 80%, at least 85%, at least 90%, at least 95%, at least 97%, or 100% identical to the amino acid sequence of SEQ ID NO: 9, 10, 12, 17, 22, 27, 32, 37, 50, 51, 56, 57, 62, or 63.

30. The polypeptide of claim 23, further comprising a multimerization domain.

31. A polypeptide comprising:
(i) at least two antigen-binding domains that bind Siglec-8 and a multimerization domain; or
(ii) at least three antigen-binding domains that bind Siglec-8.

32-37. (canceled)

38. The polypeptide of claim 30, wherein the multimerization domain is an antibody Fc region.

39. (canceled)

40. The polypeptide of claim 38, wherein the antibody Fc region is a human IgG1 Fc region or comprises an amino acid sequence selected from the group consisting of SEQ ID NO: 44, 45, 65, 66, 67, or 68.

41-43. (canceled)

44. The polypeptide of claim 38, wherein the polypeptide comprises the structure VHH-VHH-Fc, VHH-Fc-VHH, VHH-VHH-VHH-Fc, or VHH-VHH-Fc-VHH.

45. The polypeptide of claim 44, comprising an amino acid sequence at least 80%, at least 85%, at least 90%, at least 95%, at least 96%, at least 97%, at least 98%, at least 99%, or 100% identical to the amino acid sequence of SEQ ID NO: 71.

46-47. (canceled)

48. A protein comprising two or more polypeptides of claim 30 multimerized under physiological conditions via the multimerization domain.

49. (canceled)

50. A protein comprising two polypeptides of claim 38 dimerized under physiological conditions via the dimerization domain.

51-58. (canceled)

59. A pharmaceutical composition comprising the protein of claim 50, and a pharmaceutically acceptable carrier.

60. An isolated nucleic acid that encodes the polypeptide of claim 23.

61. A vector comprising the nucleic acid of claim 60.

62. A host cell comprising the nucleic acid of claim 60.

63. A host cell that expresses the polypeptide of claim 23.

64. A method of producing a polypeptide or protein, the method comprising incubating the host cell of claim 62 under conditions for expression of the polypeptide or protein.

65. (canceled)

66. A method of treating an eosinophilic disorder or a mast cell disorder, an inflammatory disease or condition, or an allergic condition, the method comprising administering to a subject in need thereof a therapeutically effective amount of the pharmaceutical composition of claim 59.

67-73. (canceled)

74. A method of depleting eosinophils in a subject, the method comprising administering to the subject a therapeutically effective amount of the protein of claim 50.

75. A method of killing an eosinophil, the method comprising contacting the eosinophil with a natural killer (NK) cell in the presence of the polypeptide or protein of claim 50.

76. A method of killing an eosinophil, the method comprising contacting the eosinophil with a macrophage in the presence of the protein of claim 50.
